SEQUENCE LISTING (19 sequences)

SEQ ID NO: 1 - protein, 5 aa, synthetic construct
TYPIE

SEQ ID NO: 2 - protein, 17 aa, synthetic construct
NFHPYNDDTKYNEKFQG

SEQ ID NO: 3 - protein, 11 aa, synthetic construct
ENYGSHGGFVY

SEQ ID NO: 4 - protein, 12 aa, synthetic construct
RASSSVISSYLH

SEQ ID NO: 5 - protein, 7 aa, synthetic construct
STSNLAS

SEQ ID NO: 6 - protein, 9 aa, synthetic construct
QQYNSYPLT

SEQ ID NO: 7 - protein, 120 aa, synthetic construct
QVQLQQSGAELVKPGASVKMSCKAFGYTFTTYPIEWMKQNHGKSLEWIGNFHPYNDDTKYNEKFKGKAKLTVEKSSTTVYLELSRLTSDDSAVYYCARENYGSHGGFVYWGQGTLVTVSG

SEQ ID NO: 8 - protein, 120 aa, synthetic construct
QVQLVQSGAEVKKPGASVKVSCKAFGYTFTTYPIEWMRQAPGKGLEWIGNFHPYNDDTKYNEKFQGRVTLTVDKSSTTVYMELSSLRSEDTAVYYCARENYGSHGGFVYWGQGTLVTVSS

SEQ ID NO: 9 - protein, 120 aa, synthetic construct
QVQLVQSGAEVKKPGASVKVSCKAFGYTFTTYPIEWVRQAPGKGLEWMGNFHPYNDDTKYNEKFQGRVTMTVDKSTTTVYMELSSLRSEDTAVYYCARENYGSHGGFVYWGQGTLVTVSS

SEQ ID NO: 10 - protein, 120 aa, synthetic construct
QVQLVQSGSELKKPGASVKVSCKAFGYTFTTYPIEWMRQAPGQGLEWIGNFHPYNDDTKYNEGFTGRFVLSVDKSSTTVYLQISSLKAEDTAVYYCARENYGSHGGFVYWGQGTLVTVSS

SEQ ID NO: 11 - protein, 120 aa, synthetic construct
QVQLVQSGSELKKPGASVKVSCKAFGYTFTTYPIEWVRQAPGQGLEWMGNFHPYNDDTKYNEGFTGRFVFSVDKSVTTVYLQISSLKAEDTAVYYCARENYGSHGGFVYWGQGTLVTVSS

SEQ ID NO: 12 - protein, 108 aa, synthetic construct
ENVLTQSPAIMSASPGEKVTMTCRASSSVISSYLHWYQQKSGASPKLWIYSTSNLASGVPDRFSGSGSGTSYSLTISSVEAEDAATYYCQQYNSYPLTFGAGTKLEIK

SEQ ID NO: 13 - protein, 108 aa, synthetic construct
ENQLTQSPSSLSASVGDRVTITCRASSSVISSYLHWYQQKPGKAPKLLIYSTSNLASGVPSRFSGSGSGTDYTLTISSLQPEDFATYYCQQYNSYPLTFGGGTKLEIK

SEQ ID NO: 14 - protein, 108 aa, synthetic construct
ENQLTQSPSSLSASVGDRVTITCRASSSVISSYLHWYQQKPGKAPKLLIYSTSNLASGVPSRFSGSGSGTDYTLTISSLQPEDFATYYCQQYRGYPLTFGGGTKLEIK

SEQ ID NO: 15 - protein, 108 aa, synthetic construct
ENVLTQSPATLSLSPGERATLSCRASSSVISSYLHWYQQKPGQAPRLLIYSTSNLASGIPARFSGSGSGTDYTLTISSLEPEDFAVYYCQQYNSYPLTFGGGTKLEIK

SEQ ID NO: 16 - protein, 108 aa, synthetic construct
ENVLTQSPATLSLSPGERATLSCRASSSVISSYLHWYQQKPGQAPRLLIYSTSNRATGIPARFSGSGSGTDYTLTISSLEPEDFAVYYCQQYNSYPLTFGGGTKLEIK

SEQ ID NO: 17 - protein, 225 aa, synthetic construct
THTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 450 aa, synthetic construct
QVQLVQSGAEVKKPGASVKVSCKAFGYTFTTYPIEWMRQAPGKGLEWIGNFHPYNDDTKYNEKFQGRVTLTVDKSSTTVYMELSSLRSEDTAVYYCARENYGSHGGFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGDSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 215 aa, synthetic construct
ENQLTQSPSSLSASVGDRVTITCRASSSVISSYLHWYQQKPGKAPKLLIYSTSNLASGVPSRFSGSGSGTDYTLTISSLQPEDFATYYCQQYNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0312743 A1
(43) Pub. Date: Oct. 5, 2023

(54) ENGINEERED PD-1 ANTIBODIES AND USES THEREOF

(71) Applicant: MiroBio Limited, Oxford (GB)

(72) Inventors: Christopher Paluch, Oxford (GB); Lynne Murray, Oxford (GB)

(21) Appl. No.: 18/056,410

(22) Filed: Nov. 17, 2022

Related U.S. Application Data
(60) Provisional application No. 63/281,404, filed on Nov. 19, 2021.

(51) Int. Cl. C07K 16/28; C12N 15/63; A61P 37/06

(52) U.S. Cl. CPC C07K 16/2896; A61P 37/06; C12N 15/63; C07K 2317/565; C07K 2317/622; C07K 2317/732; C07K 2317/92

(57) ABSTRACT

In some aspects, provided herein are antibodies that bind to PD-1. Antibodies provided herein, in some cases, agonize PD-1 signaling. Antibodies provided herein, in some cases, have modified Fc region. In other aspects, provided herein are compositions, methods of use, methods of making, and kits relating to antibodies that bind to PD-1.

### CROSS REFERENCE

This application claims the benefit of U.S. Provisional Pat. Application No. 63/281,404, filed Nov. 19, 2021, which is incorporated herein by reference in its entirety.

### REFERENCE TO A SEQUENCE LISTING

The instant application contains a Sequence Listing which has been submitted electronically in XML format and is hereby incorporated by reference in its entirety. Said XML copy, created on Nov. 17, 2022, is named 56270_718201_SL.xml and is 19,479 bytes in size.

### SUMMARY

Disclosed herein, in some aspects, is a method of suppressing an immune cell that expresses Programmed death 1 (PD-1), comprising contacting the immune cell with an antibody that specifically binds to PD-1 and agonizes PD-1 signaling in the immune cell, wherein the antibody comprises an Fc region that comprises an amino acid substitution, and wherein the amino acid substitution results in reduced antibody-dependent cellular cytotoxicity (ADCC) against a PD-1 expressing regulatory T cell in the subject compared to a parent molecule that lacks the amino acid substitution, and wherein the antibody has the same or higher agonistic effect on PD-1 signaling in the immune cell compared to the parent molecule.

Disclosed herein, in some aspects, is a method of suppressing an immune cell that expresses Programmed death 1 (PD-1), comprising contacting the immune cell with an antibody that specifically binds to PD-1 and that enhances interaction of the PD-1 on the surface of the immune cell with PD-L1. In some cases, the antibody comprises an Fc region, and wherein the Fc region comprises an amino acid substitution. In some cases, the amino acid substitution results in reduced antibody-dependent cellular cytotoxicity (ADCC) against a PD-1 expressing regulatory T cell in the subject compared to a parent molecule that lacks the amino acid substitution, and wherein the antibody has the same or higher agonistic effect on PD-1 signaling in the immune cell compared to the parent molecule.

In some embodiments of the method, the ADCC against the PD-1 expressing regulatory T cell is reduced as determined by a natural killer cell activation assay as described in Example 7.

In some embodiments of the method, the antibody does not lead to significant ADCC against the PD-1 expressing regulatory T cell, as determined by a natural killer cell activation assay as described in Example 7.

In some embodiments of the method, the antibody does not activate natural killer (NK) cells.

In some embodiments of the method, the antibody comprises a heavy chain that comprises a heavy chain variable region, and a light chain that comprises a light chain variable region. In some embodiments of the method, the heavy chain variable region comprises a complementarity determining region (CDR) comprising the sequence as set forth in one or more of SEQ ID NOs: 1-3, with 0 to 3 amino acid modifications.

In some embodiments of the method, the Fc region is derived from an IgG1 and comprises aspartic acid (D) at position 238 numbered according to EU index.

Disclosed herein, in some aspects, is a method of suppressing an immune cell that expresses Programmed death 1 (PD-1), comprising contacting the immune cell with an antibody that comprises a heavy chain, a light chain, and an Fc region, wherein: (i) the heavy chain comprises a heavy chain variable region that comprises a CDR comprising the sequence as set forth in one or more of SEQ ID NOs: 1-3, with 0 to 3 amino acid modifications; (ii) the light chain comprises a light chain variable region that comprises a CDR comprising the sequence as set forth in one or more of SEQ ID NOs: 4-6, with 0 to 3 amino acid modifications; and (iii) the Fc region is derived from an IgG1 and comprises aspartic acid (D) at position 238 numbered according to EU index.

In some embodiments of the method, the light chain variable region comprises a CDR comprising the sequence as set forth in one or more of SEQ ID NOs: 4-6, with 0 to 3 amino acid modifications.

In some embodiments of the method, the heavy chain variable region comprises heavy chain complementarity determining region 1 (CDRH1), CDRH2, and CDRH3, and wherein CDRH1, CDRH2, and CDRH3 comprise the sequence as set forth in SEQ ID NOs: 1-3, respectively, with 0 to 3 amino acid modifications.

In some embodiments of the method, the light chain variable region comprises light chain complementarity determining region 1 (CDRL1), CDRL2, and CDRL3, and wherein CDRL1, CDRL2, and CDRL3 comprise the sequence as set forth in SEQ ID NOs: 4-6, respectively, with 0 to 3 amino acid modifications.

In some embodiments of the method, the heavy chain variable region comprises CDRH1, CDRH2, and CDRH3, and wherein CDRH1, CDRH2, and CDRH3 comprise the sequence as set forth in SEQ ID NOs: 1-3, respectively.

In some embodiments of the method, the light chain variable region comprises CDRL1, CDRL2, and CDRL3, and wherein CDRL1, CDRL2, and CDRL3 comprise the sequence as set forth in SEQ ID NOs: 4-6, respectively.

In some embodiments of the method, the heavy chain variable region comprises a sequence that has at least 80%, 85%, 90%, 95%, or 99%, or 100% identity to the sequence as set forth in any one of SEQ ID NOs: 7-11.

In some embodiments of the method, the light chain variable region comprises a sequence that has at least 80%, 85%, 90%, 95%, or 99%, or 100% identity to the sequence as set forth in any one of SEQ ID NOs: 12-16.

In some embodiments of the method, the heavy chain comprises a sequence that has at least 80%, 85%, 90%, 95%, or 99%, or 100% identity to the sequence as set forth in SEQ ID NO: 18.

In some embodiments of the method, the light chain comprises a sequence that has at least 80%, 85%, 90%, 95%, or 99%, or 100% identity to the sequence as set forth in SEQ ID NO: 19.

In some embodiments of the method, the Fc region is derived from a human IgG1. In some embodiments of the method, the Fc region comprises a sequence that has at least 80%, 85%, 90%, 95%, or 99%, or 100% identity to the sequence as set forth in SEQ ID NO: 17.

In some embodiments of the method, the heavy chain variable region and the light chain variable region form a structure selected from the group consisting of: scFv, sc(Fv)2, dsFv, Fab, Fab′, (Fab′)2 and a diabody.

In some embodiments of the method, the heavy chain variable region and the light chain variable region form a single-chain variable fragment (ScFv) that is operably linked to the Fc region.

In some embodiments of the method, the antibody is selected from the group consisting of: a human antibody, a humanized antibody, a chimeric antibody, and a multispecific antibody. In some embodiments of the method, the antibody is monoclonal.

In some embodiments of the method, the antibody decreases activation of the immune cell by at least about 10%, 15%, 20%, 25%, 30%, 40%, or 50%.

In some embodiments of the method, the antibody decreases activation of the immune cell by from about 10% to 50%, 10% to 40%, 10% to 30%, 10% to 20%, 10% to 15%, 20% to 50%, 20% to 40%, or 20% to 30%.

In some embodiments of the method, the immune cell comprises a T cell, a B cell, or a macrophage. In some embodiments of the method, the immune cell comprises an antigen-specific T cell.

In some embodiments of the method, the Fc region selectively binds to FcyR2B. In some embodiments of the method, the antibody binds to human FcyR2B with a KD of less than 5 µM, 4 µM, 3 µM, or 2 µM, as determined by surface plasmon resonance at 37° C. In some embodiments of the method, the antibody binds to human FcyR2A (131R allotype) with a KD of more than 5 µM or 10 µM, as determined by surface plasmon resonance at 37° C. In some embodiments of the method, the antibody binds to human FcyR2A (131R allotype) with a KD of at least 15 µM, as determined by surface plasmon resonance at 37° C. In some embodiments of the method, the antibody binds to human FcyR2A (131H allotype) with a KD of at least 50 µM, as determined by surface plasmon resonance at 37° C. In some embodiments of the method, the antibody binds to human FcyR2A (131H allotype) with a KD of at least 80 µM, as determined by surface plasmon resonance at 37° C. In some embodiments of the method, a ratio of binding affinity of the antibody to human FcyR2B versus binding affinity of the antibody to human FcyR2A (131R allotype) is at least 2:1, 3:1, 4:1, 5:1, or 6:1. In some embodiments of the method, a ratio of binding affinity of the antibody to human FcyR2B versus binding affinity of the antibody to human FcyR2A (131R allotype) is at least 6:1. In some embodiments of the method, a ratio of binding affinity of the antibody to human FcyR2B versus binding affinity of the antibody to human FcyR2A (131R allotype) is about 6:1. In some embodiments of the method, a ratio of binding affinity of the antibody to human FcyR2B versus binding affinity of the antibody to human FcyR2A (131H allotype) is at least 10:1, 15:1, 20:1, 40:1, or 50:1. In some embodiments of the method, a ratio of binding affinity of the antibody to human FcyR2B versus binding affinity of the antibody to human FcyR2A (131H allotype) is at least 40:1. In some embodiments of the method, a ratio of binding affinity of the antibody to human FcyR2B versus binding affinity of the antibody to human FcyR2A (131H allotype) is about 40:1. In some embodiments of the method, the ratio is determined by surface plasmon resonance at 37° C.

Disclosed herein, in some aspects, is an isolated antibody that specifically binds to Programmed death 1 (PD-1) and agonizes PD-1 signaling, wherein the antibody comprises a heavy chain, a light chain, and an Fc region, wherein the heavy chain comprises a heavy chain variable region, wherein the light chain comprises a light chain variable region, wherein the Fc region comprises an amino acid substitution that results in reduced antibody-dependent cellular cytotoxicity (ADCC) against a PD-1 expressing regulatory T cell compared to a parent molecule that lacks the substitution, and wherein the antibody has the same or higher agnostic effect on PD-1 signaling in an immune cell compared to the parent molecule.

Disclosed herein, in some aspects, is an isolated antibody that specifically binds to Programmed death 1 (PD-1), wherein the antibody comprises a heavy chain, a light chain, and an Fc region, wherein the heavy chain comprises a heavy chain variable region, wherein the light chain comprises a light chain variable region, and wherein the antibody enhances interaction of PD-1 expressed on the surface of an immune cell with PD-L1. In some cases of the antibody, the Fc region comprises an amino acid substitution that results in reduced antibody-dependent cellular cytotoxicity (ADCC) against a PD-1 expressing regulatory T cell in the subject compared to a parent molecule that lacks the amino acid substitution, and wherein the antibody has the same or higher agnostic effect on PD-1 signaling in an immune cell compared to the parent molecule.

In some embodiments of the antibody, the heavy chain variable region comprises a CDR comprising the sequence as set forth in one or more of SEQ ID NOs: 1-3, with 0 to 3 amino acid modifications. In some embodiments of the antibody, the light chain variable region comprises a CDR comprising the sequence as set forth in one or more of SEQ ID NOs: 4-6, with 0 to 3 amino acid modifications. In some embodiments of the antibody, the Fc region is derived from an IgG1 and comprises aspartic acid (D) at position 238 numbered according to EU index.

Disclosed herein, in some aspects, is an isolated antibody that specifically binds to Programmed death 1 (PD-1), wherein the antibody comprises a heavy chain, a light chain, and an Fc region, wherein the heavy chain comprises a heavy chain variable region, wherein the light chain comprises a light chain variable region, and wherein: (i) the heavy chain variable region comprises a CDR comprising the sequence as set forth in one or more of SEQ ID NOs: 1-3, with 0 to 3 amino acid modifications; (ii) the light chain variable region comprises a CDR comprising the sequence as set forth in one or more of SEQ ID NOs: 4-6, with 0 to 3 amino acid modifications; and (iii) the Fc region is derived from an IgG1 and comprises aspartic acid (D) at position 238 numbered according to EU index.

In some embodiments of the antibody, the antibody enhances interaction of PD-1 expressed on the surface of an immune cell with PD-L1.

In some embodiments of the antibody, the interaction between PD-1 and PD-L1 is enhanced as determined by an assay as described in Example 10.

In some embodiments of the antibody, the antibody induces reduced antibody-dependent cellular cytotoxicity (ADCC) against a PD-1 expressing regulatory T cell compared to an otherwise same molecule that comprises an Fc region of the IgG1, and wherein the antibody has the same or higher agnostic effect on PD-1 signaling in an immune cell compared to the otherwise same molecule. In some embodiments of the antibody, the ADCC against the PD-1 expressing regulatory T cell is reduced as determined by a natural killer cell activation assay as described in Example 7. In some embodiments of the antibody, the antibody does not lead to significant ADCC against the PD-1 expressing regulatory T cell, as determined by a natural killer cell activation assay as described in Example 7.

In some embodiments of the antibody, the antibody does not activate natural killer (NK) cells.

In some embodiments of the antibody, the heavy chain variable region comprises heavy chain complementarity determining region 1 (CDRH1), CDRH2, and CDRH3, and wherein CDRH1, CDRH2, and CDRH3 comprise the sequence as set forth in SEQ ID NOs: 1-3, respectively, with 0 to 3 amino acid modifications.

In some embodiments of the antibody, the light chain variable region comprises light chain complementarity determining region 1 (CDRL1), CDRL2, and CDRL3, and wherein CDRL1, CDRL2, and CDRL3 comprise the sequence as set forth in SEQ ID NOs: 4-6, respectively, with 0 to 3 amino acid modifications.

In some embodiments of the antibody, the heavy chain variable region comprises CDRH1, CDRH2, and CDRH3, and wherein CDRH1, CDRH2, and CDRH3 comprise the sequence as set forth in SEQ ID NOs: 1-3, respectively.

In some embodiments of the antibody, the light chain variable region comprises CDRL1, CDRL2, and CDRL3, and wherein CDRL1, CDRL2, and CDRL3 comprise the sequence as set forth in SEQ ID NOs: 4-6, respectively.

In some embodiments of the antibody, the heavy chain variable region comprises a sequence that has at least 80%, 85%, 90%, 95%, or 99%, or 100% identity to the sequence as set forth in any one of SEQ ID NOs: 7-11.

In some embodiments of the antibody, the light chain variable region comprises a sequence that has at least 80%, 85%, 90%, 95%, or 99%, or 100% identity to the sequence as set forth in any one of SEQ ID NOs: 12-16.

In some embodiments of the antibody, the heavy chain comprises a sequence that has at least 80%, 85%, 90%, 95%, or 99%, or 100% identity to the sequence as set forth in SEQ ID NO: 18.

In some embodiments of the antibody, the light chain comprises a sequence that has at least 80%, 85%, 90%, 95%, or 99%, or 100% identity to the sequence as set forth in SEQ ID NO: 19.

In some embodiments of the antibody, the Fc region is derived from a human IgG1. In some embodiments of the antibody, the Fc region comprises a sequence that has at least 80%, 85%, 90%, 95%, or 99%, or 100% identity to the sequence as set forth in SEQ ID NO: 17.

In some embodiments of the antibody, the heavy chain variable region and the light chain variable region form a structure selected from the group consisting of: scFv, sc(Fv)2, dsFv, Fab, Fab′, (Fab′)2 and a diabody.

In some embodiments of the antibody, the antibody comprises a heavy chain and a light chain, wherein the heavy chain comprises the heavy chain variable region operably linked to the Fc region, and wherein the light chain comprises the light chain variable region.

In some embodiments of the antibody, the heavy chain variable region and the light chain variable region form a single-chain variable fragment (ScFv) that is operably linked to the Fc region.

In some embodiments of the antibody, the antibody is a humanized antibody.

In some embodiments of the antibody, the antibody is a human antibody.

In some embodiments of the antibody, the antibody is selected from the group consisting of: a human antibody, a humanized antibody, a chimeric antibody, and a multispecific antibody.

In some embodiments of the antibody, the antibody is monoclonal.

In some embodiments of the antibody, the antibody binds human PD-1 with a KD of less than 200 nM, 100 nM, 80 nM, 60 nM, or 40 nM, as determined by surface plasmon resonance (SPR) at 37° C.

In some embodiments of the antibody, the antibody binds human PD-1 with a KD of less than 60 nM as determined by surface plasmon resonance (SPR) at 37° C.

In some embodiments of the antibody, the antibody binds human PD-1 with a KD of less than 40 nM as determined by surface plasmon resonance (SPR) at 37° C.

In some embodiments of the antibody, the antibody binds cynomolgus PD-1 with a KD of less than 5000 nM, 4000 nM, 2000 nM, 1000 nM, 800 nM, 600 nM, 500 nM, 400 nM, 300 nM, or 200 nM as determined by surface plasmon resonance (SPR) at 37° C.

In some embodiments of the antibody, the antibody binds cynomolgus PD-1 with a KD of less than 600 nM as determined by surface plasmon resonance (SPR) at 37° C.

In some embodiments of the antibody, the antibody binds cynomolgus PD-1 with a KD of less than 300 nM as determined by surface plasmon resonance (SPR) at 37° C.

In some embodiments of the antibody, the antibody agonizes human PD-1 expressed on the surface of an immune cell.

In some embodiments of the antibody, the immune cell is a T cell.

In some embodiments of the antibody, binding of the antibody to human PD-1 expressed on the surface of an immune cell decreases proliferation of the cell relative to a comparable immune cell not bound by the antibody. In some embodiments of the antibody, the cell is a T cell. In some embodiments of the antibody, the decrease in cell activation is measured by an NFAT-reporter assay described in Example 4. In some embodiments of the antibody, the decrease in cell activation is measured by a Tetanus Toxoid activation assay or a viral peptide activation assay described in Example 5. In some embodiments of the antibody, the decrease in cell proliferation is measured by an anti-CD3/28 activation assay described in Example 6. In some embodiments of the antibody, the decrease in cell proliferation is measured when the immune cell is in proximity of PD-L1 expressing cells. In some embodiments of the antibody, the decrease in cell proliferation is measured by an assay described in Example 8. In some embodiments of the antibody, the decrease in cell proliferation is measured in vitro or in vivo. In some embodiments of the antibody, the decrease in cell proliferation is at least about 10%, 15%, 20%, 25%, 30%, 40%, or 50%. In some embodiments of the antibody, the decrease in cell proliferation is from about 10% to 50%, 10% to 40%, 10% to 30%, 10% to 20%, 10% to 15%, 20% to 50%, 20% to 40%, or 20% to 30%.

In some embodiments of the antibody, the Fc region selectively binds to FcyR2B. In some embodiments of the antibody, the antibody binds to human FcyR2B with a KD of less than 5 µM, 4 µM, 3 µM, or 2 µM, as determined by surface plasmon resonance at 37° C. In some embodiments of the antibody, the antibody binds to human FcyR2B with a KD of at least 2 µM, 1 µM, 800 nM, 600 nM, 500 nM, 400 nM, 300 nM, 200 nM, 100 nM, 80 nM, 60 nM, 50 nM, 40 nM, 30 nM, 20 nM, 10 nM, or 5 nM. In some embodiments of the antibody, the antibody binds to human FcyR2B with a KD of 200 nM to 5 µM, 400 nM to 4 µM, 500 nM to 3.5 µM, 800 nM to 3 µM, 1 µM to 5 µM, 1 µM to 4.5 µM, 1 µM to 4 µM, 1 µM to 3.5 µM, 1 µM to 3 µM, 1 µM to 2.5 µM, or 1 µM to 2 µM. In some embodiments of the antibody, the antibody binds to human FcyR2A (131R allotype) with a KD of more than 5 µM or 10 µM, as determined by surface plasmon resonance at 37° C. In some embodiments of the antibody, the antibody binds to human FcyR2A (131R allotype) with a KD of at least 15 µM, as determined by surface plasmon resonance at 37° C. In some embodiments of the antibody, the antibody binds to human FcyR2A (131H allotype) with a KD of at least 50 µM,as determined by surface plasmon resonance at 37° C. In some embodiments of the antibody, the antibody binds to human FcyR2A (131H allotype) with a KD of at least 80 µM, as determined by surface plasmon resonance at 37° C. In some embodiments of the antibody, a ratio of binding affinity of the antibody to human FcyR2B versus binding affinity of the antibody to human FcyR2A (131R allotype) is at least 2:1, 3:1, 4:1, 5:1, or 6:1. In some embodiments of the antibody, a ratio of binding affinity of the antibody to human FcyR2B versus binding affinity of the antibody to human FcyR2A (131R allotype) is at least 6:1. In some embodiments of the antibody, a ratio of binding affinity of the antibody to human FcyR2B versus binding affinity of the antibody to human FcyR2A (131R allotype) is about 6:1. In some embodiments of the antibody, a ratio of binding affinity of the antibody to human FcyR2B versus binding affinity of the antibody to human FcyR2A (131H allotype) is at least 10:1, 15:1, 20:1, 40:1, or 50:1. In some embodiments of the antibody, a ratio of binding affinity of the antibody to human FcyR2B versus binding affinity of the antibody to human FcyR2A (131H allotype) is at least 40:1. In some embodiments of the antibody, a ratio of binding affinity of the antibody to human FcyR2B versus binding affinity of the antibody to human FcyR2A (131H allotype) is about 40:1. In some embodiments of the antibody, the ratio is determined by surface plasmon resonance at 37° C.

Disclosed herein, in some aspects, is an isolated nucleic acid that comprises one or more nucleotide sequences encoding polypeptides capable of forming the antibody disclosed herein.

Disclosed herein, in some aspects, is a vector that comprises one or more nucleotide sequences encoding polypeptides capable of forming the antibody disclosed herein.

Disclosed herein, in some aspects, is a host cell comprising one or more nucleic acid molecules encoding the amino acid sequence of a heavy chain and a light chain which when expressed are capable of forming the antibody disclosed herein.

Disclosed herein, in some aspects, is a method, comprising culturing the host cell disclosed herein under conditions for production of the antibody.

Disclosed herein, in some aspects, is a method, comprising: (a) providing a host cell comprising one or more nucleic acid molecules encoding the amino acid sequence of a heavy chain and a light chain which when expressed are capable of forming the antibody disclosed herein; (b) culturing the host cell expressing the encoded amino acid sequence; and (c) isolating the antibody.

Disclosed herein, in some aspects, is an immunoconjugate comprising the antibody disclosed herein conjugated with an agent.

Disclosed herein, in some aspects, is a pharmaceutical composition comprising a therapeutically effective amount of the antibody disclosed herein or the immunoconjugate disclosed herein, and at least one pharmaceutically acceptable excipient.

Disclosed herein, in some aspects, is a pharmaceutical composition for use in treating a disease or condition, comprising a therapeutically effective amount of the antibody disclosed herein or the immunoconjugate disclosed herein, and at least one pharmaceutically acceptable excipient.

Disclosed herein, in some aspects, is a kit comprising the antibody disclosed herein or the immunoconjugate disclosed herein in a container.

In some cases of the kit, the kit further comprises an informational material containing instructions for use of the antibody disclosed herein or the immunoconjugate disclosed herein.

Disclosed herein, in some aspects, is a method of treating a disease or condition in a subject in need thereof, comprising administering to the subject a therapeutically effective amount of the antibody disclosed herein or immunoconjugate disclosed herein, or administering to the subject the pharmaceutical composition disclosed herein. In some cases of the method, the disease or condition comprises a disease or condition associated with PD-1. In some cases, the disease or condition comprises acute disseminated encephalomyelitis (ADEM), Addison’s disease, allergy, alopecia areata, amyotrophic lateral sclerosis, ANCA vasculitis, ankylosing spondylitis, anti-phospholipid syndrome, asthma, atopic dermatitis, autoimmune haemolytic anaemia, autoimmune hepatitis, autoimmune pancreatitis, autoimmune polyendocrine syndrome, Behcet’s disease, bullous pemphigoid, cerebral malaria, chronic inflammatory demyelinating polyneuropathy, coeliac disease, Crohn’s disease, Cushing’s Syndrome, dermatitis herpetiformis, dermatomyositis, diabetes mellitus type 1, eosinophilic granulomatosis with polyangiitis, gallbladder disease, graft versus host disease, Graves’ disease, Guillain-Barre syndrome, Hashimoto’s thyroiditis, Hidradenitis Suppurativa, IgG4-related disease, inflammatory bowel disease (IBD), inflammatory fibrosis, irritable bowel syndrome, juvenile arthritis, Kawasaki disease, leukemia, lupus nephritis, lyme arthritis, lymphoma, lymphoproliferative disorders, meningoencephalitis, multiple sclerosis, myasthenia gravis, myeloma, non-radiographic axial spondyloarthritis (nr-AxSpA), neuromyelitis optica, osteoarthritis, pelvic inflammatory disease, pemphigus, peritonitis, Pilonidal disease, polymyositis, primary biliary cholangitis, primary sclerosing cholangitis, psoriasis, psoriatic arthritis, rheumatoid arthritis, sarcoidosis, Sjögren’s syndrome, systemic lupus erythematosus, systemic sclerosis, Takayasu’s arteritis, temporal arteritis, transplant rejection, transverse myelitis, ulcerative colitis, uveitis, vasculitis, vitiligo and Vogt-Koyanagi-Harada Disease. In some cases, the subject is a human subject.

Disclosed herein, in some aspects, is a method of downregulating an immune response in a subject, comprising administering the subject the antibody disclosed herein, administering to the subject the immunoconjugate disclosed herein, or administering to the subject the pharmaceutical composition disclosed herein.

Disclosed herein, in some aspects, is a method of suppressing an immune cell that expresses PD-1, comprising contacting the immune cell with the antibody disclosed herein or immunoconjugate disclosed herein. In some cases, the immune cell comprises a T cell, a B cell, or a macrophage. In some cases, the immune cell comprises an antigen-specific T cell. In some cases, the subject is a human subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A includes a schematic (left) that shows an aAPC (artificial antigen presenting cell) expressing FcR, and an opposing cell membrane that presents PD-1 and TCR (T cell receptor), and a graph (right) that demonstrates in the presence of FcR-expressing aAPC, treatment with exemplary antibody Clone 19 mIgG1 led to inhibition of T cells, as indicated by the decrease in luciferase signal, while treatment of mIgG1 isotype control did not significantly affect T cell activation. In this experiment, NFAT-luciferase reporter Jurkat cells were co-cultured with stimulator cells that expressed mouse FcyR2B with increasing concentration of anti-PD-1 Clone 19 mIgG1 or isotype control, and luminescence was measured as a readout of T cell activation.

FIG. 1B includes a schematic of an aAPC (artificial antigen presenting cell) not expressing FcR, and an opposing cell membrane that presents PD-1 and TCR (T cell receptor; left), and a graph (right) that shows that in the presence of an aAPC not expressing FcR, treatment with exemplary antibody Clone 19 mIgG1 had no effect on T cell activation, as shown by the steady quantity in luciferase signal, similar to the effect following treatment with mIgG1 isotype control. In this experiment, the same assay as the experiment shown in FIG. 1A was performed using stimulator cells that do not express any Fc receptor.

FIG. 2A shows a graph that demonstrates the effect of exemplary antibodies on T cell activation, as determined by NFAT signal in a Jurkat reporter assay. The figure shows that treatment of cells with all PD-1 antibodies and P238D mutated versions of humCL19v1 (see Table 2), but not isotype control, significantly suppressed T cell activation; no significant difference was detected between the wildtype IgG1 version of humCL19v1 antibody and the P238D mutated version of humCL19v antibody. In this experiment, PD-1 expressing NFAT-luciferase Jurkat reporter cells were co-cultured for 6 hours with human FcyR2B expressing stimulator cells in the presence of various anti-PD-1 antibodies at a single concentration of 10 µg/ml, then NFAT activity was measured by luminescence quantification.

FIG. 2B shows a graph that demonstrates the effect of exemplary antibodies on T cell activation, as determined by NFAT signal in another T cell reporter assay in which human HEK293T stimulator cells expressing an anti-CD3 “T cell stimulator” were used to stimulate activation of Jurkat T cells. The graph shows that treatment of cells with P238D mutated version of humCL19v1, but not its P238D isotype control, significantly suppressed T cell activation.

FIG. 3A shows a graph that demonstrates the effect of exemplary antibodies on T cell activation, as determined by IFNγ release in a tetanus toxoid (TT) activation assay. IFNγ release by peripheral blood mononuclear cells following the tetanus toxoid activation assay in the presence of PD-L½ blocking antibodies, was significantly more suppressed following treatment with PD-1 antibodies and P238D mutated versions, as compared to IgG1 isotype control treatment. In this experiment, human PBMCs from 6 healthy donors were stimulated with Tetanus Toxoid in the presence of various PD-1 antibodies at a single dose of 1 µg/ml. IFNg production was assessed after 96 hours by ELISA of supernatants, and for each donor data was normalized to the IFNg level in cells stimulated in the absence of a test antibody. * p<0.05 vs. isotype control using one way ANOVA.

FIG. 3B shows a graph that demonstrates the effect of exemplary antibodies on T cell activation, as determined by IFNγ production in a viral peptide activation assay. IFNγ production by peripheral blood mononuclear cells following stimulation by CEF HLA Class I peptides in the presence of Brefeldin A, was significantly more suppressed following treatment with P238D mutated humCL19v1, as compared to IgG1 isotype control treatment.

FIG. 4 shows a graph demonstrating the effect of exemplary antibodies on CD25 expression, following induction of CD25 by anti-CD3 and anti-CD28 stimulation of peripheral blood mononuclear cells. Unlike isotype control, P238D mutated humCL19v1 antibody, like IgG1 antibodies, effectively suppressed primary T cell activation (CD25) expression. In this experiment, human PBMCs from 3 healthy donors were stimulated with soluble anti-CD3 and anti-CD28 antibodies in the presence of various PD-1 antibodies at a single dose of 1 µg/ml. CD25 expression on CD4 T cells was assessed after 72 hours by flow cytometry, and for each donor data was normalized to the CD25 expression level in cells stimulated in the absence of a test antibody. * p<0.05 vs. isotype control using one way ANOVA.

FIG. 5 shows in vitro degranulation by natural killer cells (antibody-dependent cellular cytotoxicity, or ADCC) induced by co-culture with regulatory T cells in a 1:5 ratio of purified NK cells to regulatory T cells in the presence of PD-1 antibody. Unlike IgG1 isotype control, all IgG1 isotype anti-PD-1 antibodies, but not P238D mutated PD-1 antibody (humCL19v1 P238D), led to significant ADCC killing of regulatory T cells, thus degranulation of the cells. In this experiment, 20,000 healthy donor NK cells were incubated with T regulatory cells, with the indicated antibodies at 1ug/ml. Data is shown from two separate studies with one Treg donor and 3 different NK cell donors per study. Each different icon represents a different NK cell donor, with NK cell degranulation normalized to the no antibody setting for that donor. *p<0.05 vs isotype control using 1-way ANOVA.

FIG. 6 shows only humCLV19v1 P238D PD-1 antibody was able to inhibit T cell activation in the presence of high PD-L1 while all other anti-PD-1 agonist antibodies did not, as determined by NFAT signal in a Jurkat reporter assay. When PD-L1 expressing cells containing a T-cell stimulator construct were incubated with PD-1 expressing Jurkat reporter cells to test the impact of the P238D mutated humCL19v1 in comparison to the other PD-1 antibodies, only the mutated P238D PD-1 antibody, significantly suppressed T cell activation. In this experiment, PD-1 expressing Jurkat reporter cells were co-cultured with PD-L1 expressing stimulator cells in the presence of various PD-1 antibodies and T cell activation was assessed by luciferase production.

FIGS. 7A-7E are graphs demonstrating the effect of exemplary PD-1 agonistic antibodies on T cell activation in RA PBMC, fibroblast co-cultures, as measured by CD25 expression (FIG. 7A), ICOS expression (FIG. 7B), IFNγ (FIG. 7C, “IFNg” in the figure), IL-17F (FIG. 7D), and TNFα (FIG. 7E, “TNFa” in the figure), respectively. In this experiment, PBMCs from patients with RA were co-cultured with fibroblast like synoviocytes and stimulated with anti-CD3 and anti-CD28 in the presence of different PD-1 antibodies or isotype control. Cells and supematants were assessed at 72 hours. CD25 and ICOS expression on CD4 T cells was assessed by flow cytometry. Levels of IFNg, IL-17F and TNFa in culture supernatants were assessed by cytometric bead array. Each symbol represents a different PBMC donor, normalized to the no antibody setting for that donor. *p<0.05 vs isotype control using 1-way ANOVA.

FIG. 8 is a graph demonstrating the effect of exemplary PD-1 agonistic antibodies on the binding of PDL1-Fc to PD-1 expressing Jurkat cells pre-incubated with various PD-1 antibodies. Only humCL19v1 P238D was shown to increase binding of PDL1-Fc to the PD-1 expressing Jurkat cells. In this experiment, PD-1 expressing Jurkat cells were pre-incubated for one hour on ice with 10 µg/ml then stained with increasing concentrations of AF647 conjugated PDL1-Fc.

FIGS. 9A-9D demonstrate that genes downregulated by PD-1 agonist are associated with autoimmunity. FIG. 9A is a graph showing the activation of Jurkat T cells in the absence of PD-L1, as measured by Luciferase activity, but in the presence of exemplary PD-1 agonistic antibodies (clone 19 mIgG1) or isotype control. PD-1 expressing Jurkat reporter cells were co-cultured with FcR expressing stimulator cells in the presence of 5 µg/ml clone 19 mIgG1 or isotype control and a portion of cells from each well was taken to assess luciferase production as a readout of T cell activation. FIG. 9B shows representative flow cytometry plots of activated Jurkat cells before and after magnetic selection. Jurkat cells were separated from stimulator cells by negative selection using magnetic beads and the purity of purified Jurkats was assessed by flow cytometry. FIG. 9C is a volcano plot that shows differentially expressed genes for Jurkats activated in the presence of PD-1 agonist vs isotype control, by bulk RNA sequencing of purified Jurkat cells with GeneWiz. FIG. 9D shows the signature of genes significantly downregulated by PD-1 agonism. The genes were mapped on the EBI GWAS catalogue, to identify enrichment of genes associated with different traits.

FIGS. 10A-10D are graphs showing the effect of exemplary PD-1 agonist antibody, Clone 19, in a mouse model of SLE, as measured by total anti-Histone IgG level (FIG. 10A), level of anti-dsDNA IgG (FIG. 10B) in the serum on day 35 after cell transfer, Tfh cell frequency (CXCR5+ICOS+ as a percent of total CD4s) in the spleen on day 35 (FIG. 10C), and spleen weight at time of study termination on day 35 (FIG. 10D). The level of anti-dsDNA IgG was assessed by ELISA and quantified as arbitrary units using a standard curve of pooled sera.

FIGS. 11A-11B show that exemplary PD-1 agonist antibody, Clone 19, prevents the expansion of Tfh cells rather than depleting Tfh cells in a mouse model of SLE. FIG. 11A shows the Tfh cell frequency in the spleen at day 30, and FIG. 11B shows spleen weight at day 30 after dosing with clone 19 at day 0, day 14 or day 28 after immune cell transfer.

FIGS. 12A-12B show that exemplary PD-1 agonist antibody, Clone 19, inhibits the delayed type hypersensitivity response in mice. FIG. 12A shows the effects of Clone 19 on keyhole limpet hemocyanin (KLH)-induced delayed type hypersensitivity (DTH). Mice were immunized with KLH antigen on Day 0, one hour after treatment with 10 mg/kg Clone 19 or isotype control antibody, and then challenged intradermally in one ear on Day 5. The difference in biopsy weight between challenged and unchallenged ear in different treatment groups was measured on Day 6 is shown in the figure. FIG. 12B shows the results of another experiment where mice were treated similarly but with various doses of Clone 19. Each dot represents an individual mouse. * above groups represents p<0.05 vs. isotype control using Kruskal-Wallis, Dunn’s multiple comparisons test.

FIGS. 13A-13E show that exemplary PD-1 agonist antibody, humCL19v1 P238D, ameliorates symptoms of graft-versus-host disease in a mouse model. Irradiated mice were injected with human peripheral blood mononuclear cells (PBMCs), and then received treatment with 10 mg/kg of humCL19v1 P238D or P238D mutated hIgG1 isotype on day 0, and on days 7, 14 and 21 after PBMC injection. humCL19v1 P238D was shown to significantly reduce spleen weights (FIG. 13A), human immune cell expansion in spleen (FIG. 13B) and liver (FIG. 13C), and serum inflammatory cytokine levels (FIG. 13D) compared to isotype control. FIG. 13E shows that humCL19v1 P238D also reduced CD4 and CD8 cytokine production on a per cell basis, as assessed by intracellular flow cytometry of human immune cells in the liver and spleen.

### DETAILED DESCRIPTION

In some aspects, disclosed herein is an antibody that specifically binds to Programmed death 1 (PD-1) and agonizes PD-1 signaling. In some cases, the PD-1 antibody disclosed herein can act as an agonist of PD-1, thereby modulating immune responses regulated by PD-1.

In some cases, the agonist anti-PD-1 antibody disclosed herein comprises a Fc region that has an amino acid substitution, which results in reduced antibody-dependent cellular cytotoxicity (ADCC) against a PD-1 expressing regulatory T cell in the subject compared to a parent molecule that lacks the Fc region amino acid substitution, but maintains or enhances the antibody’s agonistic effect on PD-1 signaling as compared to the parent molecule. In some cases, the Fc region amino acid substitution of the anti-PD-1 antibody disclosed herein results in enhanced binding selectivity to FcʏR2B, e.g., having a relatively higher binding affinity to the FcʏR2B as compared to other types of Fc receptors and the difference in the binding affinity being higher than the parent molecule that lacks the amino acid substitution. The terms “FcγR2B,” “FcgR2B,” “FcgammaR2B,” and “FcγRIIB” are used herein interchangeably and refer to the same subtype of Fc receptor.

Without wishing to be bound by a certain theory, the Fc region amino acid substitution of the anti-PD-1 antibody disclosed herein can enhance binding selectivity of the antibody to FcyR2B. In humans there is one inhibitory Fc gamma receptor (FcyR2B) whilst the other Fc gamma receptors may all deliver immune activating signals (e.g., FcγR1, FcyR2A, FcyR3A and FcyR3B). These activating FcRs can contribute to antibody dependent cellular cytotoxicity (ADCC) and antibody dependent cellular phagocytosis (ADCP), which can result in depletion of target expressing cells. It is thought that increasing the selectivity of Fc binding to FcyR2B can enhance the effectiveness of PD-1 agonist antibodies disclosed herein at suppressing immune responses without eliciting inflammatory FcR signaling, and without depleting PD-1 expressing regulatory T cells. Furthermore, in some cases, selective binding to FcyR2B can promote bidirectional inhibitory signaling through PD-1 on the PD-1 expressing cell and through FcyR2B on the FcyR2B expression cell, which can strengthen the immunosuppressive effect of the antibody. Conversely, in some cases, very high affinity for FcyR2B can adversely impact antibody half-life due to turnover of the receptor in liver sinusoidal epithelial cells (Ganesan et al. J Immunol. 2012 Nov 15;189(10):4981-8. doi: 10.4049/jimmunol.1202017) as demonstrated by the FcyR2B enhanced IgG1 antibody XmAb7195 which binds to FcyR2B with a K_{D} of 7.74 nM (Chu et al. J Allergy Clin Immunol. 2012 Apr;129(4):1102-15. doi: 10.1016/j.jaci.2011.11.029.) and was reported by Xencor to have an average in vivo half-life of 3.9 days in a phase 1a trial (American Thoracic Society (ATS) 2016 International Conference in San Francisco, CA - A6476: Poster Board Number 407), compared to an average half-life of around 21 days for a wildtype IgG1 (Morell et al. J Clin Invest. 1970;49(4):673-680. doi: 10.1172/JCI106279). Therefore, whilst selectivity for FcyR2B and sufficient binding to support agonism might be desirable for a PD-1 agonist antibody, excessively high affinity for FcyR2B might be undesirable in therapeutic context as the potential, consequential shortened half-life would likely necessitate more frequent dosing. Without wishing to be bound by a certain theory, the Fc region amino acid substitution of the anti-PD-1 antibody disclosed herein can enhance binding selectivity of the antibody to FcʏR2B, while avoiding excessively high affinity for FcʏR2B and preserving a desirable half-life of the antibody.

In some cases, the agonist anti-PD-1 antibodies disclosed are more efficacious than current antibodies at promoting inhibitory signaling toward immune cells and/or the immune system, downregulating immune cell responses. In some cases, the PD-1 antibodies disclosed herein enhance binding of PD-1 to PD-L1. In some cases, the PD-1 antibodies disclosed herein promote PD-1 signaling in an immune cell even in proximity of PD-L1. The PD-1 antibodies disclosed herein can be particularly useful in the treatment of immune system mediated, and/or PD-1 associated disorders, or diseases generated by aberrant immune pathologies or having cancerous origins. PD-1 associated disorders can include disorders that manifest dyseregulated PD-1 expression and/or activity in one or more types of immune cells as one of the symptoms, or are caused by dysregualtin of PD-1 expression and/or activity in one or more types of immune cells.

In some aspects, disclosed herein are methods, systems, pharmaceutical compositions, compositions, method of treatments, kits, and methods of manufacturing that relate to PD-1 antibodies.

It is to be understood that one, some, or all of the properties of the various embodiments described herein may be applied to any aspect unless the content clearly dictates otherwise. Furthermore, that the various embodiments may be combined to form other embodiments of the present invention. These and other aspects of the invention will become apparent to one of skill in the art. These and other embodiments of the invention are further described by the detailed description that follows.

### Definitions

The terms “agonist,” “agonistic,” “agonize,” and other grammatical equivalents, as used herein, refer to or relate to an agent that can bind to a receptor or any other protein target, and activate or enhance an activity of, or help initiate activation of, the receptor or protein target. In some cases, an agonist can promote the receptor or other protein target that it binds to, to induce a biological response, e.g., signal transduction or other changes in cellular activities. As used herein, a PD-1 agonist antibody (or antibody fragment) refers to an antibody (or antibody fragment) that binds to PD-1 expressed on the surface of an immune cell and enhances its inhibitory signal to the immune cell, including without limitation T cells, macrophages and/or B cells.

In the present disclosure, wherever aspects are described herein with the language “comprising,” otherwise analogous aspects described in terms of “consisting of” and/or “consisting essentially of” are also provided. All definitions herein described whether specifically mentioned or not, should be construed to refer to definitions as used throughout the specification and attached claims.

Throughout the specification and attached claims, the singular form “a”, “an” and “the” include plural references unless the context clearly dictates otherwise. For example, the term “a cell” includes a plurality of cells, including mixtures thereof.

In the present disclosure, one, some, or all of the properties of the various embodiments described herein may be applied to any aspect unless the content clearly dictates otherwise. Furthermore, that the various embodiments may be combined to form other embodiments of the present invention. These and other aspects of the invention will become apparent to one of skill in the art. These and other embodiments of the invention are further described by the detailed description herein.

Throughout the specification and attached claims, and unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of ordinary skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain, CDRs and framework regions (FRs), of an antibody follow, unless otherwise indicated, the Kabat definition as set forth in Kabat et al. Sequences of Proteins of Immunological Interest, 5th Ed. Public Health Service, National Institutes of Health, Bethesda, MD. (1991).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of within up to 20%, up to 10%, up to 5%, or up to 1% of a given value. Alternatively, particularly with respect to biological systems or processes, the term can mean within an order of magnitude, e.g., within 5-fold, or within 2-fold, of a value. Where particular values are described in the application and claims, unless otherwise stated the term “about” meaning within an acceptable error range for the particular value should be assumed.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides as described herein are based upon an antibody, the polypeptides can occur as single chains or associated chains.

The term “amino acid” refers to natural, unnatural, and synthetic amino acids, including but not limited to both the D or L optical isomers, and amino acid analogs and peptidomimetics. Standard single or three letter codes are used to designate amino acids.

A “variant” when applied to a protein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity compared with the reference biologically active protein or any ranges in between the at least 70% and 99% . As used herein, the term “biologically active protein moiety” includes proteins modified deliberately, as for example, by site directed mutagenesis, synthesis of the encoding gene, insertions, or accidentally through mutations.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′—O—methyl—, 2′—O—allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda MD)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

A “host cell” includes an individual cell or cell culture that can be or has been a recipient for vector(s) comprising exogenous polynucleotides. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. Kabat et al., Sequences of Proteins of Immunological Interest, 5th Ed. Public Health Service, National Institutes of Health, Bethesda, Md., 1991. The Fc region of an immunoglobulin generally comprises two constant domains, CH2 and CH3.

A “native sequence Fc region” comprises an amino acid sequence identical to the amino acid sequence of an Fc region found in nature. A “variant Fc region” comprises an amino acid sequence which differs from that of a native sequence Fc region by virtue of at least one amino acid modification, yet retains at least one effector function of the native sequence Fc region. In some cases, the variant Fc region has at least one amino acid substitution compared to a native sequence Fc region or to the Fc region of a parent polypeptide, e.g., from about one to about ten amino acid substitutions, e.g., from about one to about five amino acid substitutions in a native sequence Fc region or in the Fc region of the parent polypeptide. In some cases, the variant Fc region herein possesses at least about 80% sequence identity with a native sequence Fc region and/or with an Fc region of a parent polypeptide. In some cases, the variant Fc region herein possesses at least about 90% sequence identity with a native sequence Fc region and/or with an Fc region of a parent polypeptide. In some cases, the variant Fc region herein possesses at least about 95%, at least about 96%, at least about 97%, at least about 98%, at least about 99% sequence identity and sequence identity between said ranges with a native sequence Fc region and/or with an Fc region of a parent polypeptide.

An “individual” or a “subject” is a mammal, e.g., a human. Mammals also include, but are not limited to, farm animals, sport animals, pets, primates, horses, dogs, cats, mice and rats.

As used herein, “vector” means a construct, which is capable of delivering and expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried. An effective amount of an active agent may be administered in a single dose or in multiple doses. A therapeutically effective amount of antibody ranges from about 0.001 to about 25 mg/kg body weight, e.g., from about 0.01 to about 25 mg/kg body weight, from about 0.1 to about 20 mg/kg body weight, or from about 1 to about 10 mg/kg. The dosage may be adjusted, as necessary, to suit observed effects of the treatment and/or as most effective to provide a cure, prevention, control symptoms and the like as determined by one of ordinary skills in the art. The appropriate dose is chosen based on clinical indications by a treating physician or person of skill in the art. A component may be described herein as having at least an effective amount, or at least an amount effective to produce a desired result, such as that associated with a particular goal or purpose, such as any described herein. The desired therapeutic result herein can include, without limitation, treating, alleviating, or curing a disorder, cancer, an immune-associated disease, a PD-1 associated disorder, and/or any symptoms from immune-related pathologies and the like as described in this specification and or appended claims.

As used herein, “pharmaceutically acceptable carrier” or “pharmaceutical acceptable excipient” includes any material which, when combined with an active ingredient, allows the ingredient to retain biological activity and is non-reactive with the subject’s immune system. Examples include, but are not limited to, any of the standard pharmaceutical carriers such as a phosphate buffered saline solution, water, emulsions such as oil/water emulsion, and various types of wetting agents. Exemplary diluents for aerosol or parenteral administration are phosphate buffered saline or normal (0.9%) saline. In some cases, compositions comprising such carriers are formulated by well-known conventional methods (see, for example, Remington’s Pharmaceutical Sciences, 18th edition, A. Gennaro, ed., Mack Publishing Co., Easton, PA, 1990; and Remington, The Science and Practice of Pharmacy 20th Ed. Mack Publishing, 2000).

Throughout the specification and attached claims, the methods and systems of this disclosure as described herein may employ, unless otherwise indicated, conventional techniques and descriptions of molecular biology (including recombinant techniques), cell biology, biochemistry, microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Green, Molecular Cloning: A Laboratory Manual, 4th Edition (2012) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 6^{th} Ed., W.H. Freeman Pub., New York (2012); R.I. Freshney, Culture of Animal Cells: A Manual of Basic Technique and Specialized Applications, 6^{th} Ed., Wiley-Blackwell (2010); and Berg et al., Biochemistry, 5^{th} Ed., W.H. Freeman Pub., New York (2002), all of which are herein incorporated by reference in their entirety for all purposes. Before the present compositions, research tools and systems and methods are described, it is to be understood that this disclosure is not limited to the specific systems and methods, compositions, targets and uses described, as such may, of course, vary. It is also to be understood that the terminology used herein is for the purpose of describing particular aspects only and is not intended to limit the scope of the present disclosure, which will be limited only by appended claims.

The term “anti-PD-1 antibody” or molecule as used herein, unless otherwise specified, refers to either an antibody or a binding fragment thereof, which is capable of specific binding to PD-1.

In the present disclosure, an “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. The term as used herein, includes an immunoglobulin molecule that specifically binds to an antigen and comprises an FcR binding site which may or may not be functional. As used in the disclosure, the term encompasses not only intact polyclonal or monoclonal antibodies, but also fragments thereof (such as Fab, Fab′, F(ab′)2, diabodies) Fv fragments and single chain (ScFv) mutants that contain an antigen recognition site or antigen binding site and have ability to bind to an antigen. Antigen-binding antibody or immunoglobulin fragments are well known in the art; such fragment can have a functional or non-functional Fc receptor binding site. Further as used herein, the term is not limited only to intact polyclonal or monoclonal antibodies, multispecific antibodies such as bispecific, or polyspecific antibodies generated from at least two intact antibodies, chimeric antibodies, humanized antibodies, single-chain, chimeric, synthetic, recombinant, hybrid, mutated, grafted antibodies, human antibodies, and any other modified immunoglobulin molecule comprising an antigen binding site so long as the antibodies exhibit the desired biological activity.

There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Unless dictated otherwise by contextual constraints the antibodies of the invention can be from one of these classes or subclasses of antibodies. Heavy-chain constant domains that correspond to the different classes of antibodies are typically denoted by the corresponding lower-case Greek letter α, δ, ε, γ, and µ, respectively. Light chains of the antibodies from any vertebrate species can be assigned to one of two clearly distinct types, called kappa (κ) and lambda (λ), based on the amino acid sequences of their constant domains.

Throughout the specification and appended claims, “Fc receptor” and “FcR” describe a receptor that binds to the Fc region of an antibody. FcRs are reviewed in Ravetch and Kinet, 1991, Ann. Rev. Immunol., 9:457-92; Capel et al., 1994, Immunomethods, 4:25-34; and de Haas et al., 1995, J. Lab. Clin. Med., 126:330-41. “FcR” also includes the neonatal receptor, FcRn, which is responsible for the transfer of maternal IgGs to the fetus (Guyer et al., 1976, J. Immunol., 117:587; and Kim et al., 1994, J. Immunol., 24:249).

Wherever used herein, “monoclonal antibody” refers to an antibody obtained from a population of substantially homogeneous antibodies. In general, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

Wherever used herein “antibody-dependent cell cytotoxicity” and “ADCC” refer to a cell-mediated reaction in which nonspecific cytotoxic cells that express Fc receptors (FcRs) (e.g., natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337, or those described in Example 7 of the present disclosure. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

“Complement dependent cytotoxicity” and “CDC” refer to the lysing of a target in the presence of complement. The complement activation pathway is initiated by the binding of the first component of the complement system (C1q) to a molecule (e.g., an antibody) complexed with a cognate antigen. To assess complement activation, a CDC assay, e.g., as described in Gazzano-Santoro et al., J. Immunol. Methods, 202:163 (1996), may be performed.

An antibody that “specifically binds” to an epitope is a term well understood in the art, and methods to determine such specific binding are also well known in the art. A molecule is said to exhibit “specific binding” if it reacts or associates more frequently, more rapidly, with greater duration and/or with greater affinity with a particular cell, protein or substance than it does with alternative cells, proteins or substances. An antibody “specifically binds” or “preferentially binds” to a target if it binds with greater affinity, avidity, more readily, and/or with greater duration than it binds to other substances. For example, an antibody that specifically or preferentially binds to PD-1 is an antibody that binds this epitope with greater affinity, avidity, more readily, and/or with greater duration than it binds to other epitopes. As a further example, an antibody (or other moiety) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “fragment” when applied to a protein, is a truncated form of a native biologically active protein that may or may not retain at least a portion of the therapeutic and/or biological activity. Herein, the terms “antibody fragment,” “antigen-binding fragment thereof”, and “fragment thereof” when used with reference to an antibody, are used interchangeably.

### Sequence Identity

The sequence identity with respect to the anti-PD-1 antibody sequences or any other amino acid sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured. In some embodiments, percent identity is determined with respect to the full length of a noted reference sequence, such as a sequence provided herein. For example, sequence comparison between two amino acid sequences (or a shorter length thereof) of the present disclosure may be carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{\text{X}}{\text{Y}} \times 100 \%$$

where X is the number of amino acid residues scored as identical matches by the sequence alignment program BLAST in that program’s alignment of A and B, and where Y is the total number of amino acid residues in A or B, whichever is shorter.

Two polynucleotide or polypeptide sequences are said to be “identical” if the sequence of nucleotides or amino acids in the two sequences is the same when aligned for maximum correspondence. Comparisons between two sequences are typically performed by comparing the sequences over a comparison window to identify and compare local regions of sequence similarity.

### Programmed Cell Death 1 (PD-1)

In some aspects, provided herein are compositions and methods related to antibodies or antigen-binding fragments thereof that bind to and agonize PD-1, a receptor that can be present on the surface of activated lymphocytes including T cells, natural killer cells, B cells, and monocytes and on the surface of myeloid cells. The PD-1 pathway can be a critical immune checkpoint to regulate the response of PD-1 expressing immune cells.

Without wishing to be bound by a certain theory, activation of the PD-1 pathway can lead to inhibition of immune cell activation. Antibodies that block PD-1 signaling are used in cancer patients to promote anti-tumor immune responses.

Programmed cell death protein 1 (PD-1 or CD279) is an immunoglobulin superfamily (IgSF) protein and member of the B7-CD28 family. It may consist of a single extracellular IgV-like domain, a single pass transmembrane region and a cytoplasmic tail that contains an ITIM and an ITSM. In some cases, it is monomeric on the cell surface as it lacks the extracellular cysteine found in CD28, CTLA4 and ICOS that allows these molecules to form covalent homodimers. In some cases, PD-1 is also expressed on cells across the immune system including CD4 T-cells, CD8 T-cells, B cells, NKT cells, monocytes, macrophages and dendritic cells. It can be briefly upregulated on acutely activated T-cells and persistently upregulated on exhausted T-cells. PD-1 can bind to two ligands called PD-L1 (PD-L1, CD279, or B7-H1) and PDL-2 (CD273 or B7-DC), each of which contains two IgSF domains in their extracellular region. PD-L1 can be constitutively expressed on professional antigen presenting cells (APCs) such as DCs, macrophages and B cells and can be induced on non-hematopoietic cells during inflammation to limit tissue damage, but is also often upregulated on cancer cells enabling them to dampen anti-tumor immune responses.

On binding to its ligands PD-1′s intracellular tyrosine motifs can become phosphorylated and the phosphorylated ITSM can recruit the protein phosphatase SHP2 (and possibly SHP1). Once recruited to the cell surface SHP2 negatively regulates cell signaling by dephosphorylating the ITAMs of activatory receptors (in particular CD28) and other downstream mediators of activatory signaling. As well as suppressing activation of T cells, PD-1 signaling can also play a role in the generation of regulatory T-cells (Tregs). The term “PD-1 signaling” as used herein can refer to one or more of the phosphorylation of the PD-1′s intracellular tyrosine motifs, the recruitment of the protein phosphatase SHP2 and/or SHP1, or dephosphorylation of the ITAMs of activatory receptors or other downstream mediators of activatory signaling. An antibody disclosed herein, in some cases, promotes PD-1 signaling, e.g., promotes one or more of phosphorylation of the intracellular tyrosine motifs of the PD-1 molecule the antibody binds to, or a PD-1 molecule that the antibody does not bind to but bind to another PD-1 molecule that is expressed on the same cell surface, recruitment of SHP2 and/or SHP1, or dephosphorylation of the ITAMs of activatory receptors or other downstream mediators of activatory signaling.

In some aspects, provided herein are antibodies, compositions, uses thereof, and methods of making the same that can circumvent some of the aforementioned and other problems known in the art that are associated with existing anti-PD-1 antibodies. In some embodiments, provided herein are PD-1 agonist antibodies that are able to trigger PD-1 signaling to bind to PD-L1 on effector T cells without depleting PD-1 expressing regulatory T cells, or with minimal depleting effects on PD-1 expressing regulatory T cells.

Without wishing to be bound by a certain theory, in autoimmune disease, PD-L1 expression can be upregulated by the inflammatory environment (Keir et al. 2008)(Garcia-Diaz et al. 2017), which raises the possibility that PD-1 is already fully engaged under these pathological conditions and thus providing a limited scope for additional benefit of an agonist antibody. However, in some aspects, provided herein are PD-1 agonist antibodies that can provide an additional inhibitory signal even in the presence of receptor engagement by its natural ligand PD-L1. In some embodiments, the PD-1 agonist antibodies disclosed herein have inhibitory effects on PD-1-expressing immune cells that are in proximity of PD-L1-expressing cells or PD-L1 itself, are in contact with PD-L1, or have PD-1 receptors engaged with PD-L1.

### Antibody Sequence

In the present disclosure, provided herein are compositions, therapeutics, kits, vectors, nucleic acid sequences, manufacturing, culturing and/or methods for producing an PD-1 agonist antibody or an antigen-binding fragment or a functional fragment thereof having a mutation in the Fc region (FcR) enhances selectivity for inhibitory Fc receptor, FcyR2B (CD23B) and thereby enhances the biological effects of PD-1 activation, e.g., inhibiting the activity or proliferation of the immune cell that expresses the PD-1 molecule the antibody binds to, or promoting downregulation of PD-1 expressing immune cell responses. In some cases, a PD-1 agonist antibody enhances the interaction between PD-1 and PD-L1. In some cases, a PD-1 agonist antibody promotes the downstream signaling of PD-1 that is triggered by PD-L1 binding. In some cases, a PD-1 agonist antibody promotes the downstream signaling of PD-1 without increasing or enhancing the interaction between PD-1 and PD-L1. In some cases, a PD-1 agonist antibody activates or enhances PD-1 signaling in the absence of PD-L1 binding to PD-1.

In some embodiments, an antibody or an antigen-binding fragment (e.g., an isolated antibody) provided herein specifically binds to PD-1 and enhances interaction of PD-1 expressed on the surface of an immune cell with PD-L1.

In some embodiments, an antibody or an antigen-binding fragment (e.g., an isolated antibody) provided herein is a PD-1 antibody that comprises a heavy chain variable region, a light chain variable region, and an Fc region, and the Fc region of the PD-1 antibody comprises an amino acid substitution that enhances selectivity for inhibitory Fc receptor, FcyR2B, thereby enhancing PD-L1 mediated triggering of PD-1, and increasing the PD-⅟PD-L1 interaction resulting in reduced antibody-dependent cellular cytotoxicity (ADCC) against a PD-1 expressing regulatory T cell compared to a parent molecule that lacks the substitution.

In some embodiments, an antibody, or an antibody fragment (e.g., an isolated antibody) provided herein is a PD-1 antibody that comprises a heavy chain variable region, a light chain variable region, and an Fc region. In some embodiments, the heavy chain variable region of the PD-1 antibody comprises a CDR comprising a sequence with at least 80%, 85%, 90%, 95%, 96%, 97%, 98%, or 99%, or 100% sequence identity to the sequence as set forth in one or more of SEQ ID NOs: 1-3, with 0 to 3 amino acid modifications. In some embodiments, the heavy chain variable region of the PD-1 antibody comprises a CDR comprising the sequence as set forth in one or more of SEQ ID NOs: 1-3, with 0 to 3 amino acid modifications. In some embodiments, the light chain variable region of the PD-1 antibody comprises a CDR comprising a sequence with at least 80%, 85%, 90%, 95%, 96%, 97%, 98%, or 99%, or 100% sequence identity to the sequence as set forth in one or more of SEQ ID NOs: 4-6, with 0 to 3 amino acid modifications. In some embodiments, the light chain variable region of the PD-1 antibody comprises a CDR comprising the sequence as set forth in one or more of SEQ ID NOs: 4-6, with 0 to 3 amino acid modifications. In some embodiments, the Fc region of the PD-1 antibody is derived from an IgG1 molecule (e.g., a human IgG1 molecule) and comprises an amino acid substitution from proline (P) to aspartic acid (D) at position 238 numbered according to EU index.

In some cases a CDR of the heavy chain variable region of the antibody (or antigen-binding fragment, hereafter referred to as “antibody,” to represent the full length antibody or the antigen-binding fragment of an antibody) comprises a sequence with at least 80%, 85%, 90%, 95%, 96%, 97%, 98%, or 99%, or 100% sequence identity to the sequence as set forth in one or more of SEQ ID NOs: 1-3. In some cases a CDR of the heavy chain variable region of the antibody comprises the sequence as set forth in one or more of SEQ ID NOs: 1-3. In some cases the CDR of the antibody comprises the sequence as set forth in one or more of SEQ ID NOs: 1-3, with one amino acid modification. In some cases a CDR of the heavy chain variable region of the antibody comprises the sequence as set forth in one or more of SEQ ID NOs: 1-3, with two amino acid modifications. In some cases a CDR of the heavy chain variable region of the antibody comprises the sequence as set forth in one or more of SEQ ID NOs: 1-3, with three amino acid modifications.

In some cases an antibody provided herein comprises a heavy chain variable region comprising three heavy chain complementarity-determining regions (CDRH), wherein CDRH1 has an amino acid sequence with at least 80%, 85%, 90%, 95%, 96%, 97%, 98%, or 99%, or 100% sequence identity to the sequence as set forth in SEQ ID NO:1, CDRH2 has an amino acid sequence with at least 80%, 85%, 90%, 95%, 96%, 97%, 98%, or 99%, or 100% sequence identity to the sequence as set forth in SEQ ID NO:2, and CDRH3 has an amino acid sequence with at least 80%, 85%, 90%, 95%, 96%, 97%, 98%, or 99%, or 100% sequence identity to the sequence set forth in SEQ ID NO: 3, with 0 to 3 amino acid modifications. In some cases an antibody provided herein comprises a heavy chain variable region comprising three heavy chain complementarity-determining regions (CDRH), wherein CDRH1 has an amino acid sequence as set forth in SEQ ID NO:1, CDRH2 has an amino acid sequence as set forth in SEQ ID NO:2, and CDRH3 has an amino acid sequence set forth in SEQ ID NO: 3, with 0 to 3 amino acid modifications. In some cases in the heavy chain variable region of the antibody, CDRH1 has an amino acid sequence as set forth in SEQ ID NO:1, CDRH2 has an amino acid sequence as set forth in SEQ ID NO:2, and CDRH3 has an amino acid sequence set forth in SEQ ID NO: 3. In some cases in the heavy chain variable region of the antibody, CDRH1 has an amino acid sequence as set forth in SEQ ID NO:1, CDRH2 has an amino acid sequence as set forth in SEQ ID NO:2, and CDRH3 has an amino acid sequence set forth in SEQ ID NO: 3, with one amino acid modification. In some cases in the heavy chain variable region of the antibody, CDRH1 has an amino acid sequence as set forth in SEQ ID NO:1, CDRH2 has an amino acid sequence as set forth in SEQ ID NO:2, and CDRH3 has an amino acid sequence set forth in SEQ ID NO: 3, with two amino acid modifications. In some cases in the heavy chain variable region of the antibody, CDRH1 has an amino acid sequence as set forth in SEQ ID NO:1, CDRH2 has an amino acid sequence as set forth in SEQ ID NO:2, and CDRH3 has an amino acid sequence set forth in SEQ ID NO: 3, with three amino acid modifications.

In some cases an antibody provided herein comprises a light chain variable region comprising a CDR with amino acid sequence as set forth in one or more of SEQ ID NOs: 4-6, with 0 to 3 amino acid modifications. In some cases a CDR of the light chain variable region of the antibody comprises the sequence as set forth in one or more of SEQ ID NOs: 4-6. In some cases a CDR of the light chain variable region of the antibody comprises the sequence as set forth in one or more of SEQ ID NOs: 4-6, with one amino acid modification. In some cases a CDR of the light chain variable region of the antibody comprises the sequence as set forth in one or more of SEQ ID NOs: 4-6, with two amino acid modifications. In some cases a CDR of the light chain variable region of the antibody comprises the sequence as set forth in one or more of SEQ ID NOs: 4-6, with three amino acid modifications.

In some cases an antibody provided herein comprises a light chain variable region comprising three light chain complementarity-determining regions wherein CDRL1 has an amino acid sequence with at least 80%, 85%, 90%, 95%, 96%, 97%, 98%, or 99%, or 100% sequence identity to the sequence as set forth in SEQ ID NO: 4, CDRL2, has an amino acid sequence with at least 80%, 85%, 90%, 95%, 96%, 97%, 98%, or 99%, or 100% sequence identity to the sequence as set forth in SEQ ID NO: 5, and CDRL3 has an amino acid sequence with at least 80%, 85%, 90%, 95%, 96%, 97%, 98%, or 99%, or 100% sequence identity to the sequence as set forth in SEQ ID NO: 6, with 0 to 3 amino acid modifications. In some cases an antibody provided herein comprises a light chain variable region comprising three light chain complementarity-determining regions wherein CDRL1 has an amino acid sequence as set forth in SEQ ID NO: 4, CDRL2, has an amino acid sequence as set forth in SEQ ID NO: 5, and CDRL3 has an amino acid sequence as set forth in SEQ ID NO: 6, with 0 to 3 amino acid modifications. In some cases in the light chain variable region of the antibody, CDRL1 has an amino acid sequence as set forth in SEQ ID NO: 4, CDRL2, has an amino acid sequence as set forth in SEQ ID NO: 5, and CDRL3 has an amino acid sequence as set forth in SEQ ID NO: 6. In some cases in the light chain variable region of the antibody, CDRL1 has an amino acid sequence as set forth in SEQ ID NO: 4, CDRL2, has an amino acid sequence as set forth in SEQ ID NO: 5, and CDRL3 has an amino acid sequence as set forth in SEQ ID NO: 6, with one amino acid modification. In some cases in the light chain variable region of the antibody, CDRL1 has an amino acid sequence as set forth in SEQ ID NO: 4, CDRL2, has an amino acid sequence as set forth in SEQ ID NO: 5, and CDRL3 has an amino acid sequence as set forth in SEQ ID NO: 6, with two amino acid modifications. In some cases in the light chain variable region of the antibody, CDRL1 has an amino acid sequence as set forth in SEQ ID NO: 4, CDRL2, has an amino acid sequence as set forth in SEQ ID NO: 5, and CDRL3 has an amino acid sequence as set forth in SEQ ID NO: 6, with three amino acid modifications.

In some cases an antibody provided herein comprises a heavy chain variable region and a light chain variable region, the heavy chain variable region comprises three heavy chain complementarity-determining regions (CDRH), wherein CDRH1 has an amino acid sequence as set forth in SEQ ID NO: 1, CDRH2 has an amino acid sequence as set forth in SEQ ID NO:2, and CDRH3 has an amino acid sequence set forth in SEQ ID NO: 3, with 0 to 3 amino acid modifications, and the light chain variable region comprises three light chain complementarity-determining regions wherein CDRL1 has an amino acid sequence as set forth in SEQ ID NO: 4, CDRL2, has an amino acid sequence as set forth in SEQ ID NO: 5, and CDRL3 has an amino acid sequence as set forth in SEQ ID NO: 6, with 0 to 3 amino acid modifications. In some cases CDRH1 has an amino acid sequence as set forth in SEQ ID NO:1, CDRH2 has an amino acid sequence as set forth in SEQ ID NO:2, and CDRH3 has an amino acid sequence set forth in SEQ ID NO: 3, and the light chain variable region comprises three light chain complementarity-determining regions wherein CDRL1 has an amino acid sequence as set forth in SEQ ID NO: 4, CDRL2, has an amino acid sequence as set forth in SEQ ID NO: 5, and CDRL3 has an amino acid sequence as set forth in SEQ ID NO: 6.

In some cases an antibody (e.g., an isolated antibody) provided herein comprises a heavy chain variable region (VH) comprising a sequence that has at least 80%, 85%, 90%, 95%, or 99%, or 100% identity to the sequence as set forth in any one of SEQ ID NO: 7, SEQ ID NO: 8, SEQ ID NO: 9, SEQ ID NO: 10, and SEQ ID NO: 11.

In some cases an antibody (e.g., an isolated antibody or “antibody” hereafter) provided herein comprises a light chain variable region (VL) comprising a sequence that has at least 80%, 85%, 90%, 95%, or 99%, or 100% identity to the sequence as set forth in any one of SEQ ID NO: 12, SEQ ID NO: 13, SEQ ID NO: 14, SEQ ID NO: 15, and SEQ ID NO: 16. In some cases an antibody provided herein comprises a light chain variable region (VL) comprising a sequence that has at least 80% identity to the sequence as set forth in any one of SEQ ID NO: 12, SEQ ID NO: 13, SEQ ID NO: 14, SEQ ID NO: 15, and SEQ ID NO: 16. In some cases an antibody provided herein comprises a light chain variable region (VL) comprising a sequence that has at least 85% identity to the sequence as set forth in any one of SEQ ID NO: 12, SEQ ID NO: 13, SEQ ID NO: 14, SEQ ID NO: 15, and SEQ ID NO: 16. In some cases an antibody provided herein comprises a light chain variable region (VL) comprising a sequence that has at least 90% identity to the sequence as set forth in any one of SEQ ID NO: 12, SEQ ID NO: 13, SEQ ID NO: 14, SEQ ID NO: 15, and SEQ ID NO: 16. In some cases an antibody provided herein comprises a light chain variable region (VL) comprising a sequence that has at least 95% identity to the sequence as set forth in any one of SEQ ID NO: 12, SEQ ID NO: 13, SEQ ID NO: 14, SEQ ID NO: 15, and SEQ ID NO: 16. In some cases an antibody provided herein comprises a light chain variable region (VL) comprising a sequence that has at least 99% identity to the sequence as set forth in any one of SEQ ID NO: 12, SEQ ID NO: 13, SEQ ID NO: 14, SEQ ID NO: 15, and SEQ ID NO: 16. In some cases an antibody provided herein comprises a light chain variable region (VL) comprising a sequence that has at least 100% identity to the sequence as set forth in any one of SEQ ID NO: 12, SEQ ID NO: 13, SEQ ID NO: 14, SEQ ID NO: 15, and SEQ ID NO: 16. In some cases an antibody provided herein comprises a light chain variable region (VL) comprising a sequence that has at least 80% and any range in between up to 100% identity to the sequence as set forth in any one of SEQ ID NO: 12, SEQ ID NO: 13, SEQ ID NO: 14, SEQ ID NO: 15, and SEQ ID NO: 16.

In some cases an antibody (e.g., an isolated antibody) provided herein comprises a heavy chain variable region (VH) and a light chain variable region (VL), wherein VH comprises a sequence that has at least 80%, 85%, 90%, 95%, or 99%, or 100% identity to the sequence as set forth in any one of SEQ ID NO: 7, SEQ ID NO: 8, SEQ ID NO: 9, SEQ ID NO: 10, and SEQ ID NO: 11, and VL comprises a sequence that has at least 80%, 85%, 90%, 95%, or 99%, or 100% identity to the sequence as set forth in any one of SEQ ID NO: 12, SEQ ID NO: 13, SEQ ID NO: 14, SEQ ID NO: 15, and SEQ ID NO: 16. In some cases an antibody (e.g., an isolated antibody) provided herein comprises a heavy chain variable region (VH) and a light chain variable region (VL), wherein the VH comprises a sequence that has at least 80%, identity to the sequence as set forth in any one of SEQ ID NO: 7, SEQ ID NO: 8, SEQ ID NO: 9, SEQ ID NO: 10, and SEQ ID NO: 11. In some cases an antibody (e.g., an isolated antibody) provided herein comprises a heavy chain variable region (VH) and a light chain variable region (VL), wherein the VH comprises a sequence that has at least 85%, identity to the sequence as set forth in any one of SEQ ID NO: 7, SEQ ID NO: 8, SEQ ID NO: 9, SEQ ID NO: 10, and SEQ ID NO: 11. In some cases an antibody (e.g., an isolated antibody) provided herein comprises a heavy chain variable region (VH) and a light chain variable region (VL), wherein the VH comprises a sequence that has at least 90%, identity to the sequence as set forth in any one of SEQ ID NO: 7, SEQ ID NO: 8, SEQ ID NO: 9, SEQ ID NO: 10, and SEQ ID NO: 11. In some cases an antibody (e.g., an isolated antibody) provided herein comprises a heavy chain variable region (VH) and a light chain variable region (VL), wherein the VH comprises a sequence that has at least 95%, identity to the sequence as set forth in any one of SEQ ID NO: 7, SEQ ID NO: 8, SEQ ID NO: 9, SEQ ID NO: 10, and SEQ ID NO: 11. In some cases an antibody (e.g., an isolated antibody) provided herein comprises a heavy chain variable region (VH) and a light chain variable region (VL), wherein the VH comprises a sequence that has at least 99%, identity to the sequence as set forth in any one of SEQ ID NO: 7, SEQ ID NO: 8, SEQ ID NO: 9, SEQ ID NO: 10, and SEQ ID NO: 11. In some cases an antibody (e.g., an isolated antibody) provided herein comprises a heavy chain variable region (VH) and a light chain variable region (VL), wherein the VH comprises a sequence that has at least 100%, identity to the sequence as set forth in any one of SEQ ID NO: 7, SEQ ID NO: 8, SEQ ID NO: 9, SEQ ID NO: 10, and SEQ ID NO: 11. In some cases an antibody (e.g., an isolated antibody) provided herein comprises a heavy chain variable region (VH) and a light chain variable region (VL), wherein the VL comprises a sequence that has at least 80%, 85%, 90%, 95%, or 99%, or 100% identity to the sequence as set forth in any one of SEQ ID NO: 12, SEQ ID NO: 13, SEQ ID NO: 14, SEQ ID NO: 15, and SEQ ID NO: 16. In some cases an antibody (e.g., an isolated antibody) provided herein comprises a heavy chain variable region (VH) and a light chain variable region (VL), wherein the VL comprises a sequence that has at least 80%, identity to the sequence as set forth in any one of SEQ ID NO: 12, SEQ ID NO: 13, SEQ ID NO: 14, SEQ ID NO: 15, and SEQ ID NO: 16. In some cases an antibody (e.g., an isolated antibody) provided herein comprises a heavy chain variable region (VH) and a light chain variable region (VL), wherein the VL comprises a sequence that has at least 85%, identity to the sequence as set forth in any one of SEQ ID NO: 12, SEQ ID NO: 13, SEQ ID NO: 14, SEQ ID NO: 15, and SEQ ID NO: 16. In some cases an antibody (e.g., an isolated antibody) provided herein comprises a heavy chain variable region (VH) and a light chain variable region (VL), wherein the VL comprises a sequence that has at least 90%, identity to the sequence as set forth in any one of SEQ ID NO: 12, SEQ ID NO: 13, SEQ ID NO: 14, SEQ ID NO: 15, and SEQ ID NO: 16. In some cases an antibody (e.g., an isolated antibody) provided herein comprises a heavy chain variable region (VH) and a light chain variable region (VL), wherein the VL comprises a sequence that has at least 95%, identity to the sequence as set forth in any one of SEQ ID NO: 12, SEQ ID NO: 13, SEQ ID NO: 14, SEQ ID NO: 15, and SEQ ID NO: 16. In some cases an antibody (e.g., an isolated antibody) provided herein comprises a heavy chain variable region (VH) and a light chain variable region (VL), wherein the VL comprises a sequence that has at least 99%, identity to the sequence as set forth in any one of SEQ ID NO: 12, SEQ ID NO: 13, SEQ ID NO: 14, SEQ ID NO: 15, and SEQ ID NO: 16. In some cases an antibody (e.g., an isolated antibody) provided herein comprises a heavy chain variable region (VH) and a light chain variable region (VL), wherein the VL comprises a sequence that has at least 100%, identity to the sequence as set forth in any one of SEQ ID NO: 12, SEQ ID NO: 13, SEQ ID NO: 14, SEQ ID NO: 15, and SEQ ID NO: 16.

In some cases an antibody (e.g., an isolated antibody) provided herein comprises a heavy chain variable region (VH) and a light chain variable region (VL), wherein VH comprises the sequence as set forth in any one of SEQ ID NO: 7, SEQ ID NO: 8, SEQ ID NO: 9, SEQ ID NO: 10, and SEQ ID NO: 11, and VL comprises the sequence as set forth in any one of SEQ ID NO: 12, SEQ ID NO: 13, SEQ ID NO: 14, SEQ ID NO: 15, and SEQ ID NO: 16.

In particular embodiments, the heavy chain or light chain disclosed in the specification and appended claims may also comprise a constant region. If the molecule is a full-length IgG-type antibody molecule, the heavy chain may comprise three constant domains.

In one aspect, the antibody provided herein comprises an Fc region, wherein said Fc region is derived from a human IgG1. In some cases the Fc region of the antibody provided herein comprises an amino acid substitution wherein proline is replaced with an aspartic acid (D) at position 238 numbered according to EU index. In one aspect, the Fc region of the antibody provided herein comprises an amino acid substitution wherein proline is replaced with an aspartic acid (D) at position 238 numbered according to EU index, and further comprises a sequence that has at least 80%, 85%, 90%, 95%, or 99%, or 100% identity to the sequence as set forth in SEQ ID NO: 17. In other aspect, the Fc region of the antibody provided herein comprises an amino acid substitution wherein proline is replaced with an aspartic acid (D) at position 238 numbered according to EU index, and further comprises a sequence that has at least 80%, identity to the sequence as set forth in SEQ ID NO: 17. In one aspect, the Fc region of the antibody provided herein comprises an amino acid substitution wherein proline is replaced with an aspartic acid (D) at position 238 numbered according to EU index, and further comprises a sequence that has at least 80%, identity to the sequence as set forth in SEQ ID NO: 17. In one aspect, the Fc region of the antibody provided herein comprises an amino acid substitution wherein proline is replaced with an aspartic acid (D) at position 238 numbered according to EU index, and further comprises a sequence that has at least 85%, identity to the sequence as set forth in SEQ ID NO: 17. In one aspect, the Fc region of the antibody provided herein comprises an amino acid substitution wherein proline is replaced with an aspartic acid (D) at position 238 numbered according to EU index, and further comprises a sequence that has at least 90%, identity to the sequence as set forth in SEQ ID NO: 17. In one aspect, the Fc region of the antibody provided herein comprises an amino acid substitution wherein proline is replaced with an aspartic acid (D) at position 238 numbered according to EU index, and further comprises a sequence that has at least 95%, identity to the sequence as set forth in SEQ ID NO: 17. In one aspect, the Fc region of the antibody provided herein comprises an amino acid substitution wherein proline is replaced with an aspartic acid (D) at position 238 numbered according to EU index, and further comprises a sequence that has at least 99%, identity to the sequence as set forth in SEQ ID NO: 17. In one aspect, the Fc region comprises a sequence that has at least 100%, identity to the sequence as set forth in SEQ ID NO: 17.

In some cases the antibody disclosed herein comprises a heavy chain variable region, and an Fc region comprising an amino acid substitution, wherein the heavy chain variable region comprises a CDR comprising the sequence as set forth in one or more of SEQ ID NOs: 1-3, with 0 to 3 amino acid modifications. In some cases the antibody comprises a heavy chain variable region (VH) and an Fc region comprising an amino acid substitution, wherein the VH comprises a CDR comprising the sequence as set forth in one or more of SEQ ID NOs: 1-3. In some cases the agonist antibody comprises a VH and an Fc region comprising an amino acid substitution, wherein the VH comprises a CDR comprising the sequence as set forth in one or more of SEQ ID NOs: 1-3, with 1 amino acid modifications. In some cases the antibody comprises VH and an Fc region comprising an amino acid substitution, wherein the VH comprises a CDR comprising the sequence as set forth in one or more of SEQ ID NOs: 1-3, with 2 amino acid modifications. In some cases the antibody comprises a VH and an Fc region comprising an amino acid substitution, wherein the VH comprises a CDR comprising the sequence as set forth in one or more of SEQ ID NOs: 1-3, with 3 amino acid modifications.

In some cases the agonist antibody comprises a light chain variable region (VL), and an Fc region comprising an amino acid substitution, wherein the light chain variable region comprises a CDR comprising the sequence as set forth in one or more of SEQ ID NOs: 4-6, with 0 to 3 amino acid modifications. In one aspect of the embodiment, the agonist antibody comprises a VL and an Fc region comprising an amino acid substitution, wherein the VL region comprises a CDR comprising the sequence as set forth in one or more of SEQ ID NOs: 4-6. In one aspect of the embodiment, the agonist antibody comprises a VL and an Fc region comprising an amino acid substitution, wherein the VL region comprises a CDR comprising the sequence as set forth in one or more of SEQ ID NOs: 4-6 with 1 amino acid modifications. In one aspect of the embodiment, the agonist antibody comprises a VL and an Fc region comprising an amino acid substitution, wherein the VL region comprises a CDR comprising the sequence as set forth in one or more of SEQ ID NOs: 4-6 with 2 amino acid modifications. In one aspect of the embodiment, the agonist antibody comprises a VL and an Fc region comprising an amino acid substitution, wherein the VL region comprises a CDR comprising the sequence as set forth in one or more of SEQ ID NOs: 4-6 with 3 amino acid modifications.

In some cases the antibody or antigen-binding fragment disclosed herein comprises a heavy chain variable region and a light chain variable region that form a structure selected from the group consisting of: scFv, sc(Fv)2, dsFv, Fab, Fab′, (Fab′)2 and a diabody.

### Effector Functions

In some cases the antibody disclosed herein agonizes PD-1, e.g., human PD-1 expressed on the surface of an immune cell, such as a T cell, a B cell, or a macrophage. In some cases the antibody disclosed herein binds to PD-1 expressed on the surface of an effector immune cell and decreases activation and/or proliferation of the effector immune cell relative to a comparable immune cell not bound by said antibody.

In some cases the antibody disclosed herein binds to PD-1 expressed on immune cells and inhibits activation and/or proliferation of the cells. In some embodiments, the antibody disclosed herein binds to PD-1 expressed on immune cells and decreases activation and/or proliferation of the immune cell when the immune cell is in proximity of PD-L1 expressing cells. In some cases, the antibody disclosed herein has agonistic effect on PD-1 signaling in an immune cell in the presence of large amount of PD-L1 in surrounding environment. Without wishing to be bound by a certain theory, in some cases, in autoimmune disease PD-L1 expression is upregulated by the inflammatory environment (Keir et al. Annu Rev Immunol. 2008;26:677-704. doi: 10.1146/ annurev.immunol.26.021607.090331; Garcia-Diaz et al. Cell Rep. 2017 May 9;19(6):1189-1201. doi: 10.1016/j.celrep.2017.04.031), and as such, it is possible that in such inflammatory environment PD-1 is already fully engaged with surrounding PD-L1, with limited scope for additional benefit of known agonist antibodies that target PD-1. Antibodies according to some embodiments of the present disclosure can further promote PD-1 signaling in an immune cell even in proximity of PD-L1 expressing cells, suggesting that the antibody disclosed herein can be particularly useful for downregulating immune response, e.g., in the context of autoimmune diseases, and thus can be useful for treatment of disorders associated with excessive immune response, e.g., autoimmune diseases.

In some embodiments, the antibody disclosed herein binds to PD-1 expressed on immune cells and decreases activation and/or proliferation of the immune cell in the absence of PD-L1 binding to the PD-1 molecule that the antibody binds to.

In some cases the inhibitory effect of the antibody disclosed herein on activation and/or proliferation of immune cells can be measured by an NFAT-reporter assay, such as the one described in Example 4. For instance, an NFAT-reporter assay can be carried out with Jurkat T cells that are engineered to express luciferase under the control of an NFAT response-element. In some cases, Jurkat T cells expressing luciferase under the control of an NFAT response-element, are cultured with stimulator cells that are configured to stimulate the Jrukat T cells, such as murine BW5147 cells expressing an anti-CD3 ‘T cell stimulator’ (TCS) construct as previously described (Leitner et al. 2010) and expressing human FcyR2B, or HEK293T cells expressing an anti-CD3 “T cell stimulator” (TCS) construct as previously described (Leitner et al. 2010) and expressing human FcyR2B. Co-culturing the Jurkat T cells with stimulator cells for a certain period of time, plus either test antibody (e.g., exemplary PD-1 antibody according to some embodiments of the present disclosure), or isotype control, or some other control PD-1 antibodies. After incubation with the test antibody or control for a certain period of time (such as 3 hours, 6 hours, 9 hours, or 12 hours), the cells can be collected for luciferase assay. Luminescence signal can be quantified as an indicator of the activation of the Jurkat T cells.

In some cases, the inhibitory effect of the antibody disclosed herein on activation and/or proliferation of immune cells (e.g., T cells) can be measured by an immune cell activation assay (e.g., Tetanus Toxoid activation assay or viral peptide activation assay), such as those described in Example 5. For instance, immune cells, such as human peripheral blood mononuclear cells (PBMCs), can be collected and stimulated with tetanus toxoid (e.g., 0.5 µg/mL) or a viral peptide (e.g., CEF HLA Class I peptides - a commercially available pooled mixture of peptides from cytomegalovirus, Epstein-Barr virus and influenza) in the presence of PD-L½-blocking antibodies (e.g., 5 µg/mL each) and a certain concentration of a test PD-1 antibody (e.g., exemplary antibodies according to some embodiments herein), or isotype control, or some other control antibodies. Interferon (e.g., IFNγ) release from the cells (e.g., in the supernatant or cell culture medium) can be assessed by ELISA or any other appropriate method after incubation with the test antibodies for a certain period of time (e.g., 24 hours, 48 hours, 72 hours, 96 hours, or one week). Interferon release can be measured as an indicator of the activation of the immune cells. In some cases, interferon production without the stimulation treatment (e.g., tetanus toxoid treatment or viral peptide treatment) can also be deducted as unstimulated background from the interferon production with the treatment.

In some cases, the inhibitory effect of the antibody disclosed herein on activation and/or proliferation of immune cells (e.g., T cells) can be measured by an anti-CD3/28 activation assay, such as the described in Example 6. For instance, immune cells, such as human peripheral blood mononuclear cells (PBMCs), can be collected and stimulated with soluble anti-CD3 and anti-CD28 antibodies (e.g., 0.5 ng/mL final concentration of each) in the presence of a test PD-1 antibody (e.g., exemplary antibodies according to some embodiments herein), or isotype control, or some other control antibodies at a certain concentration. CD25 expression on CD4 T cells can be assessed by flow cytometry or any other method as a marker of T cell activation after incubation with the antibody for a certain period of time (e.g., 24 hours, 48 hours, 72 hours, or 96 hours).

In some cases, the inhibitory effect of the antibody disclosed herein on activation and/or proliferation of immune cells (e.g., T cells) can be measured by cell proliferation, cytokine production, chemokine production, or any other activation markers of the immune cells. The percentage of inhibition described herein is measured by normalizing the readout of the immune cell activation marker in the cells treated with the subject antibody against otherwise the same cells but treated with an isotype control or not treated with the subject antibody. In some embodiments, the antibody disclosed herein binds to PD-1 expressed on immune cells and decreases activation and/or proliferation of the immune cell for at least about 10%, 15%, 20%, 25%, 30%, 40%, or 50%.

In some cases, with respect to inhibition of immune cell activation (e.g., T cell activation), the antibody disclosed herein has an IC50 of at most about 0.5 nM, at most about 0.2 nM, at most about 0.15 nM, at most about 0.1 nM, at most about 0.09 nM, at most about 0.08 nM, at most about 0.07 nM, at most about 0.06 nM, at most about 0.05 nM, at most about 0.04 nM, at most about 0.03 nM, at most about 0.02 M, or at most about 0.01 nM. In some cases, with respect to inhibition of immune cell activation (e.g., T cell activation), the antibody disclosed herein has an IC50 of about 0.5 nM, about 0.2 nM, about 0.15 nM, about 0.1 nM, about 0.09 nM, about 0.08 nM, about 0.07 nM, about 0.06 nM, about 0.05 nM, about 0.04 nM, about 0.03 nM, about 0.02 M, or about 0.01 nM. The IC50 of the antibody disclosed herein with respect to inhibition of immune cell activation (e.g., T cell activation) can be measured in an immune cell assay, such as those described above and in the Examples.

In some cases provided herein is a method of suppressing an immune cell that express the PD-1 using the antibody disclosed herein. The method can include contacting the immune cell expressing PD-1 with the antibody, and decreasing the activation and/or proliferation of the immune cell by about 10% to 50%. In some cases, the method results in reduction of activation and/or proliferation of the immune cell by about 10% to 40%. In some cases, the method results in reduction of activation and/or proliferation of the immune cell by about 10% to 30%. In some cases, the method results in reduction of activation and/or proliferation of the immune cell by about 10% to 20%. In some cases, the method results in reduction of activation and/or proliferation of the immune cell by about 10% to 15%. In some cases, the method results in reduction of activation and/or proliferation of the immune cell by about 20% to 50%. In some cases, the method results in reduction of activation and/or proliferation of the immune cell by about 20% to 40%. In some cases, the method results in reduction of activation and/or proliferation of the immune cell by about or 20% to 30%.

In some cases the antibody disclosed herein enhances the interaction of PD-1 expressed on the surface of an immune cell with PD-L1. For instance, in some cases, the antibody disclosed herein enhances binding of PD-L1 to a PD-1 molecule that the antibody binds to. In some cases, the antibody disclosed herein enhances binding of PD-L1 to one or more PD-1 molecules on the surface of an immune cell that are not bound by the antibody, whereas the antibody binds to other PD-1 molecules on the surface of the immune cell. In one embodiment, the antibody disclosed herein enhances the interaction of PD-1 expressed on the surface of an immune cell with PD-L1, as determined by an assay, such as the one as described in Example 10. In some cases, the antibody disclosed herein enhances the interaction of PD-1 expressed on the surface of an immune cell with PD-L1 by at least 10%, 20%, 30%, 40%, 50%, 60%, 70%, 80%, 90%, 100%, 120%, 150%, 180%, 200%, 300%, 400%, 500%, or even more.

In some cases the antibodies disclosed herein have reduced induction of antibody-dependent cellular cytotoxicity (ADCC) against PD-1 expressing regulatory T cells compared to an otherwise identical molecule that comprises an unmodified Fc region of IgG1, while having the same or enhanced agonistic effect on PD-1 signaling compared to an otherwise identical molecule that comprises an unmodified Fc region of IgG1. In some cases the antibody disclosed herein, has a reduced ADCC against a PD-1 expressing regulatory T cell, as determined by a natural killer cell activation assay, such as the one described in Example 7. In some cases the antibody disclosed herein does not lead to significant ADCC against a PD-1 expressing regulatory T cell, as determined by either an in vitro assay such as described in Example 7, or in vivo when the antibody is administered to a subject. In some instances, the antibody disclosed herein does not cause natural killer cells degranulation or causes a reduced level of degranulation compared to an otherwise identical molecule that comprises an unmodified Fc region of IgG1. In some instances, the antibody disclosed herein does not lead to death of regulatory T cells or leads to a reduced level of death of regulatory T cells compared to an otherwise identical molecule that comprises an unmodified Fc region of IgG1. In some embodiments, the antibody disclosed herein does not cause natural killer cells degranulation or death of regulatory T cells or causes reduced natural killer cells degranulation or death of regulatory T cells compared to an otherwise identical molecule that comprises an unmodified Fc region of IgG1. The effect on natural killer cell degranulation and/or death of regulatory T cells can be measured in vivo or in vitro, e.g., by an assay as described in Example 7.

### Binding Affinity

The binding affinity of a humanized anti-PD-1 antibody variant for human or cynomolgus PD-1 receptor or PD-1 of another animal may be characterized by kₐ, k_{d} or K_{D}. The term “kₐ”, as used herein, is intended to refer to the rate constant for association of an antibody to an antigen. The term “k_{d} ”, as used herein, is intended to refer to the rate constant for dissociation of an antibody from the antibody/antigen complex. The term “K_{D}”, or “KD,” as used herein interchangeably, is intended to refer to the equilibrium dissociation constant of an antibody-antigen interaction. For purposes of the present disclosure, K_{D} is defined as the ratio of the two kinetic rate constants kₐ/k_{d}. The smaller the equilibrium dissociation constant the tighter the subject antibody and the PD-1 bind to each other.

In some cases the antibody disclosed herein binds human PD-1 with a K_{D} of less than 200 nM, 100 nM, 80 nM, 60 nM, or 40 nM, as determined by surface plasmon resonance (SPR) at 37° C. In one aspect, the antibody disclosed herein binds cynomolgus PD-1 with a K_{D} of less than 5000 nM, 4000 nM, 2000 nM, 1000 nM, 800 nM, 600 nM, 500 nM, 400 nM, 300 nM, or 200 nM as determined by surface plasmon resonance (SPR) at 37° C.

In some cases, the antibody or antigen-binding fragment thereof disclosed herein possesses increased binding to FcʏR2B and reduced binding to one or more activating Fcʏ receptors, such as FcʏR2A (e.g., 131R allotype or 131H allotype) or FcʏR1A, compared to the parent molecule that lacks the Fc region substitution.

In some cases, the antibody or antigen-binding fragment thereof disclosed herein possesses increased ratio of binding to FcʏR2B/ FcʏR2A (e.g., 131R allotype or 131H allotype), compared to the parent molecule that lacks the Fc region amino acid substitution. In some cases, the increased ratio of binding FcʏR2B/ FcʏR2A (e.g., 131R allotype or 131H allotype), is at least 1.1, 1.2, 1.3, 1.4, 1.5, 1.8, 2, 2.2, 2.5, 3, 3.5, 4, 5, 6, 7, 8, 9, 10, 15, 20, 25, 30, 40, 50, 60, 70, 80, 90, 100, 110, 120, 130, 140, or 150-fold compared to the parent molecule that lacks the Fc region substitution.

In some cases, the Fc region of the antibody or antigen-binding fragment thereof disclosed herein binds to FcʏR2B with a higher affinity relative to a comparable control antibody that comprises an Fc region that lacks the amino acid substitution recited above. In some cases, the antibody binds to FcʏR2B with a dissociation constant (K_{D}) of at most about 5 µM, e.g., from about 5 µM to 0.1 µM, as determined by surface plasmon resonance (SPR).

In some cases, the Fc region (FcR) of the antibody or antigen-binding fragment disclosed herein selectively binds to FcyR2B. In some cases, the antibody binds to FcʏR2B with a K_{D} of at most 5 µM, as determined by surface plasmon resonance (SPR).

In some cases, the antibody or antigen-binding fragment disclosed herein binds to human FcyR2B with a K_{D} of less than 5 µM,4 µM, 3 µM, or 2 µM, as determined by surface plasmon resonance at 37° C. In some cases the antibody or antigen-binding fragment disclosed herein binds to human FcyR2B with a K_{D} of at least 2 µM, 1 µM, 800 nM, 600 nM, 500 nM, 400 nM, 300 nM, 200 nM, 100 nM, 80 nM, 60 nM, 50 nM, 40 nM, 30 nM, 20 nM, 10 nM, or 5 nM. In some cases the antibody or antigen-binding fragment disclosed herein binds to human FcyR2B with a K_{D} of 200 nM to 5 µM, 400 nM to 4 µM, 500 nM to 3.5 µM, 800 nM to 3 µM, 1 µM to 5 µM, 1 µM to 4.5 µM, 1 µM to 4 µM, 1 µM to 3.5 µM, 1 µM to 3 µM, 1 µM to 2.5 µM,or 1 µM to 2 µM.

In some cases, the antibody binds to FcʏR2A (e.g., 131R allotype or 131H allotype) with a lower or equal affinity relative to a parental molecule, a parental molecule being the equivalent antibody that lacks the Fc substitution that confers on the antibody molecule an increased binding to and thus enhanced signaling of FcʏR2B.

In some cases, when the antibody comprises the P238D substitution the antibody binds to FcʏR2A (131R allotype) with a lower or equal affinity relative to a comparable control antibody that comprises an Fc region that comprises a proline at position 238 (EU Index).

In some cases, the antibody binds to FcʏR2A (131R allotype) with a K_{D} of more than 5 µM, as determined by surface plasmon resonance (SPR) at 37° C. In some cases, the antibody binds to FcʏR2A (131R allotype) with a K_{D} of more than 10 µM, as determined by surface plasmon resonance (SPR) at 37° C. In some cases, the antibody binds to FcʏR2A (131R allotype) with a K_{D} of at least 15 µM, as determined by surface plasmon resonance (SPR) at 37° C. In some cases, the antibody binds to FcʏR2A (131R allotype) with a K_{D} of at least 20 µM, as determined by surface plasmon resonance (SPR) at 37° C. In some cases, the antibody binds to FcʏR2A (131R allotype) with a K_{D} of from about 15 µM to 25 µM, as determined by surface plasmon resonance (SPR) at 37° C.

In some cases, the antibody binds to FcʏR2A (131H allotype) with a K_{D} of at least 50 µM, as determined by surface plasmon resonance (SPR) at 37° C. In some cases, the antibody binds to FcʏR2A (131H allotype) with a K_{D} of at least 75 µM, as determined by surface plasmon resonance (SPR) at 37° C. In some cases, the antibody binds to FcʏR2A (131H allotype) with a K_{D} of at least 80 µM,as determined by surface plasmon resonance (SPR) at 37° C. In some cases, the antibody binds to FcʏR2A (131H allotype) with a K_{D} of at least 90 µM,as determined by surface plasmon resonance (SPR) at 37° C. In some cases, the antibody binds to FcʏR2A (131H allotype) with a K_{D} of from about 50 µM to about 100 µM,as determined by surface plasmon resonance (SPR) at 37° C. In some cases, the antibody binds to FcʏR2A (131H allotype) with a K_{D} of from about 75 µM to about 125 µM,as determined by surface plasmon resonance (SPR) at 37° C.

In some cases, the antibody possesses a [K_{D} value of the antibody for FcʏR2A (131R) / K_{D} value of the antibody for FcyR2B] of 3 or more, such as at least 4, 5, 6, 7, 8, 9, or 10. Suitably, as determined by surface plasmon resonance (SPR).

In some cases, the antibody possesses a [K_{D} value of the antibody for FcyR2A(131H)]/ [K_{D} value of the antibody for FcʏR2B] of 10 or more, such as at least 15, 20, 25, 30, 35, 40, 45, or 50. Suitably, as determined by surface plasmon resonance (SPR).

In some cases, the antibody possesses a [K_{D} value of the antibody for FcʏR2A (131R) / K_{D} value of the antibody for FcyR2B] of 3 or more, such as at least 4, 5, 6, 7, 8, 9, or 10, and/or a [K_{D} value of the antibody for FcʏR2A(131H)] / [K_{D} value of the antibody for FcyR2B] of 10 or more, such as at least 15, 20, 25, 30, 35, 40, 45, or 50. Suitably, as determined by surface plasmon resonance (SPR).

In some cases, the antibody or antigen-binding fragment thereof disclosed herein possesses increased ratio of binding to FcʏR2B/ FcʏR1A, compared to the parent molecule that lacks the Fc region substitution over the wild-type sequence. In some cases, the increased ratio of binding FcʏR2B/ FcʏR1A, is at least 1.1, 1.2, 1.5, 2, 5, 10, 50, 100, 150, 200, 250-fold compared to the parent molecule that lacks the Fc region substitution.

By compared to the parent molecule that lacks the Fc region substitution it is meant compared to the antibody molecule that has the same amino acid sequence other than the amino acid recited in the claim which represents the Fc substitution relative to wildtype Fc. Thus, binding of the antibody molecule with or without the recited Fc substitution to FcʏR2B can be measured and optionally binding of the antibody molecule with or without the recited Fc substitution to an activating Fcʏ receptor, such as FcʏR2A (e.g., 131R allotype or 131H allotype) or FcʏR1A can be measured, e.g., by SPR.

In some cases, the antibody or antigen-binding fragment thereof disclosed herein has an increased ratio of [K_{D} value for binding of FcʏR1A]/[K_{D} value for binding of FcyR2B] compared to the parent molecule that lacks the Fc region substitution over the wild-type sequence. In some cases, the ratio of [K_{D} value for binding of FcʏR1A]/[K_{D} value for binding of FcyR2B] for the variant molecule is at least 1.1, 1.2, 1.5, 2, 5, 6, 7, 8, 10, 50, 100, 150, 200, 250, 300, 350, 400, 450, 500, 1000, 1500, 2000, 3000, 4000, 5000, 6000, 7000, 8000, 9000, or 10000 times the ratio of [K_{D} value for binding of FcʏR1A]/[K_{D} value for binding of FcyR2B] for the parent molecule that lacks the Fc region substitution.

In some cases, the antibody or antigen-binding fragment thereof disclosed herein has an increased ratio of [K_{D} value for binding of FcʏR2A (131R)]/[K_{D} value for binding of FcʏR2B] compared to the parent molecule that lacks the Fc region substitution over the wild-type sequence. In some cases, the ratio of [K_{D} value for binding of FcʏR2A (131R)]/[K_{D} value for binding of FcʏR2B] for the variant molecule is at least 1.1, 1.2, 1.5, 2, 5, 10, 50, or 100 times the ratio of [K_{D} value for binding of FcʏR1A]/[Kb value for binding of FcyR2B] for the parent molecule that lacks the Fc region substitution.

In some embodiments provided herein, binding affinity for each of the humanized variants of anti-PD-1 antibody to human PD-1, cynomolgus PD-1 or PD-1 of another animal is measured by surface plasmon resonance. Biacore® surface plasmon resonance (SPR) system (GE Healthcare, Chicago IL) may be used to measure binding affinity of a subject antibody. Exemplary SPR analysis systems include, but are not limited to, Biacore X100, Biacore T200, Biacore 3000 or Biacore 4000 instrument, and commercial sensor chips series. In a typical application of the Biacore systems, interaction kinetics are analyzed by monitoring the interaction as a function of time over a range of analyte concentrations, and then fitting the whole data set to a mathematical model describing the interaction. The association phase (during sample injection) contains information on both association and dissociation processes, while only dissociation occurs during the dissociation phase (after sample injection, when buffer flow removes dissociated analyte molecules). Those skilled in the art can choose or determine appropriate parameters and/or conditions for carrying out the binding affinity assay according to manufacturer’s manual. In some embodiments, the binding affinity of a subject antibody is determined by surface plasmon resonance at 37° C. In some cases the binding affinity and kinetics of the humanized antibody variants to human or cynomolgus PD-1 are determined by surface plasmon resonance (SPR) using the Biacore 8 K (Cytiva), such as disclosed in Example 2.

### Antibody Engineering

In some cases the antibody disclosed herein comprises a human antibody. In some embodiments, the antibody disclosed herein comprises a monoclonal humanized antibody, a chimeric antibody, or a multispecific antibody. In some cases the antibody disclosed herein comprises a monoclonal antibody.

An antibody embodied herein can be a monoclonal antibody, a chimeric antibody, a human or humanized antibody. The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The term “humanized antibody” is intended to refer to antibodies in which CDR sequences derived from the germline of another mammalian species, such as a mouse, have been grafted onto human framework sequences. Additional framework region modifications may be made within the human framework sequences. The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody. In some embodiments, the antibody provided herein is a monoclonal antibody.

The subject antibody can be prepared by the hybridoma process or the recombinant DNA process. As described in the method of Kohler & Milstein (Nature, 256:495 (1975)), antibody-producing cells used in the cell fusion step of preparing hybridomas are spleen cells, lymph node cells, peripheral blood leukocytes, etc. of an animal (e.g., mouse, rat, hamster, rabbit, monkey, goat) immunized with an antigen (PD-1, its partial peptide, or cells expressing them). It is also possible to use antibody-producing cells obtained by allowing an antigen to act in a culture medium on the above cells or lymphocytes isolated in advance from an unimmunized animal. As myeloma cells, publicly known various cell strains can be used. The antibody-producing cells and myeloma cells may originate in different animal species, if they are mutually fusible. In some cases, they are of the same animal species origin. Hybridomas, for example, are produced by cell fusion between spleen cells obtained from an antigen-immunized mouse and mouse myeloma cells, and subsequent screening can obtain hybridomas producing a monoclonal antibody against PD-1. The monoclonal antibody against PD-1 can be produced by a culture of the hybridomas, or from an ascitic fluid of a mammal administered the hybridomas.

In some embodiments, the antibody disclosed herein is a humanized antibody. In making humanized antibodies, the choice of framework residues can be critical in retaining high binding affinity. In principle, a framework sequence from any HuAb can serve as the template for CDR grafting; however, it has been demonstrated that straight CDR replacement into such a framework can lead to significant loss of binding affinity to the antigen. Glaser et al. (1992) J. Immunol. 149:2606; Tempest et al. (1992) Biotechnology 9:266; and Shalaby et al. (1992) J. Exp. Med. 17:217. The more homologous a HuAb is to the original muAb, the less likely that the human framework will introduce distortions into the murine CDRs that could reduce affinity. Based on a sequence homology search against an antibody sequence database, the HuAb IC4 provides good framework homology to muM4TS.22, although other highly homologous HuAbs would be suitable as well, especially kappa L chains from human subgroup I or H chains from human subgroup III. Kabat et al. (1987). Various computer programs such as ENCAD (Levitt et al. (1983) J. Mol. Biol. 168:595) are available to predict the ideal sequence for the V region. The invention thus encompasses HuAbs with different V regions. It is within the skill of one in the art to determine suitable V region sequences and to optimize these sequences. Methods for obtaining antibodies with reduced immunogenicity are also described in U.S. Pat. No. 5,270,202 and EP 699,755.

In some embodiments, the antibody disclosed herein comprises a heavy chain variable region and said light chain variable region form a structure selected from the group consisting of: scFv, sc(Fv)2, dsFv, Fab, Fab′, (Fab′)2 and a diabody.

In one aspect, the antibody disclosed herein comprises a heavy chain and a light chain, wherein the heavy chain comprises said heavy chain variable region operably linked to said Fc region, and wherein the light chain comprises said light chain variable region. In one feature, the antibody disclosed herein is a humanized antibody. In one aspect, the antibody disclosed herein is a human antibody. In another embodiment, the antibody disclosed herein is selected from the group consisting of: a human antibody, a humanized antibody, a chimeric antibody, and a multispecific antibody. In some cases the antibody disclosed herein is a monoclonal antibody.

### Humanization

In some embodiments, provided herein are antibody variants comprising any potential combinations of humanized VH and VL domains. In some embodiment, an antibody provided herein comprises humanized variants of VH of PD-1 agonist antibody comprising human framework sequences. In some embodiments, the antibody or antigen-binding fragment comprises humanized variants of VL of PD-1 agonist antibody comprising human framework sequences.

Antibodies that are humanized can retain high affinity for the antigen and other favorable biological properties. To achieve this goal, in one example, PD-1 humanized antibodies are prepared by a process of analysis of the parental sequences and various conceptual humanized products using three dimensional models of the parental and humanized sequences. Three dimensional immunoglobulin models are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, and of residues that influence the ability of the candidate immunoglobulin to bind its antigen. In this way, FR residues can be selected and combined from the consensus and import sequence so that the desired antibody characteristic, such as increased affinity for the target antigen(s), is achieved.

In some cases the variable heavy (VH) chain comprises amino acid sequence set forth in SEQ ID NO: 7. In some cases the humanized VH chain comprises human framework IGHV1-24*01. In some embodiments, the humanized VH chain comprises an amino acid sequence with at least 80%, 85%, 90%, 95%, 96%, 97%, 98%, or 99%, or 100% sequence identity to the sequence set forth in SEQ ID NO: 8. In some embodiments, the humanized VH chain comprises an amino acid sequence with at least 80%, 85%, 90%, 95%, 96%, 97%, 98%, or 99%, or 100% sequence identity to the sequence set forth in SEQ ID NO: 9. In yet another embodiment, humanized VH chain comprises human framework IGHV7-4-1*02. In some embodiments, the humanized VH chain comprises an amino acid sequence with at least 80%, 85%, 90%, 95%, 96%, 97%, 98%, or 99%, or 100% sequence identity to the sequence set forth in SEQ ID NO: 10. In some embodiments, the humanized VH chain comprises an amino acid sequence with at least 80%, 85%, 90%, 95%, 96%, 97%, 98%, or 99%, or 100% sequence identity to the sequence set forth in SEQ ID NO: 11.

In some cases the variable light (VL) chain comprises amino acid sequence set forth in SEQ ID NO: 12. In some cases humanized VL chain comprises human framework IGKV1-39*01. In some embodiments, the humanized VL chain comprises an amino acid sequence with at least 80%, 85%, 90%, 95%, 96%, 97%, 98%, or 99%, or 100% sequence identity to the sequence set forth in SEQ ID NO:13. In some cases humanized VL chain comprises human framework IGKV3-11*01. In some embodiments, the humanized VL chain comprises an amino acid sequence with at least 80%, 85%, 90%, 95%, 96%, 97%, 98%, or 99%, or 100% sequence identity to the sequence set forth in SEQ ID NO: 14. In some embodiments, the humanized VL chain comprises an amino acid sequence with at least 80%, 85%, 90%, 95%, 96%, 97%, 98%, or 99%, or 100% sequence identity to the sequence set forth in SEQ ID NO: 15. In some embodiments, the humanized VL chain comprises an amino acid sequence with at least 80%, 85%, 90%, 95%, 96%, 97%, 98%, or 99%, or 100% sequence identity to the sequence set forth in SEQ ID NO: 16.

### Mutation

In some embodiments provided herein, the PD-1 antibody as described herein may have one or more mutations or modifications with respect to a reference sequence. A mutation or modification may be a deletion, an insertion or addition, or a replacement or substitution to an amino acid residue. A “deletion” refers to a change in an amino acid sequence due to the absence of one or more amino acid residues. An “insertion” or “addition” refers to changes in an amino acid sequence resulting in the addition of one or more amino acid residues as compared to a reference sequence. A “replacement” or “substitution” refers to the replacement of one or more amino acids by different amino acids. In the context of the present disclosure, the mutations of a subject antibody or a fraction thereof with respect to a reference sequence may be determined by comparison of the subject antibody or a fraction thereof to the reference sequence. Optimal alignment of sequences for comparison may be conducted according to any of the known methods in the art.

A mutation may be identified by the mutation site. The mutation site is the position on a reference sequence where a modification, such as a deletion, an addition, or a substitution, takes place. The amino acid residues on a reference sequence are numbered from the N-terminus to the C-terminus, and the mutation site is the numbering of the amino acid residue on which a deletion, an addition, or a substitution takes place. For example, position 26 on a reference sequence is the position where the 26^{th} amino acid residue locates starting from the N-terminus.

### Antibody Conjugate

In some embodiments, an antibody or fragment thereof disclosed herein is fused to serum albumins. Fusion to serum albumins can improve the pharmacokinetics of a subject antibody as described herein. For example, the subject antibody or fragment thereof may be fused with a serum albumin. Serum albumin is a globular protein that is the most abundant blood protein in mammals. Serum albumin is produced in the liver and constitutes about half of the blood serum proteins. It is monomeric and soluble in the blood. In some embodiments, the subject antibody or fragment thereof may be fused to a serum albumin. In further embodiments, serum albumin is human serum albumin (HSA).

In some embodiments, an antibody or fragment thereof disclosed herein is fused to an albumin-binding peptide that displays binding activity to serum albumin to increase the half-life of the subject antibody or fragment thereof. Albumin-binding peptides that can be used herein include but are not limited to those described in e.g., Dennis et al., J. Biol. Chem. 277:35035-35043, 2002 and Miyakawa et al., J. Pharm. Sci. 102:3110-3118, 2013. In some embodiments, an albumin-binding peptide is fused genetically to a subject antibody or fragment thereof described herein. In further embodiments, an albumin-binding peptide is attached to a subject antibody described herein or fragment thereof through chemical means, e.g., chemical conjugation. In some embodiments, an albumin-binding peptide may be fused to the N- or C-terminus of a subject antibody or fragment thereof described herein. The C-terminus of the albumin-binding peptide may be directly fused to the N-terminus of the subject antibody through a peptide bond. Alternatively, the N-terminus of the albumin-binding peptide may be directly fused to the C-terminus of the subject antibody or fragment thereof through a peptide bond. In further embodiments, the carboxylic acid at the C-terminus of the albumin-binding peptide may be fused to an internal amino acid residue of the subject antibody or fragment thereof using conventional chemical conjugation techniques.

In some embodiments, a PD-1 antibody or fragment thereof disclosed herein is fused to a polymer, e.g., polyethylene glycol (PEG). The antibody or fragment thereof can be pegylated to, for example, increase the biological (e.g., serum) half-life of the antibody or fragment thereof. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. In some cases, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. Methods for pegylating proteins such as those disclosed in for example, EP 0 154 316 by Nishimura et al. and EP 0 401 384 by Ishikawa et al may be used. In some embodiments, a polymer, e.g., PEG, may be covalently attached to a subject antibody, or fragment thereof, described herein, either at the N- or C-terminus or at an internal location, using conventional chemical methods, e.g., chemical conjugation. Without being bound by a theory, PEG moieties may contribute to, once attached to the antibody as described herein, the water solubility, high mobility in solution, lack of toxicity and low immunogenicity, extended circulating life, increased stability, ready clearance from the body, and altered distribution in the body.

Other half-life extension technologies that may be used to increase the serum half-life of the subject antibodies, or fragment thereof, include, but are not limited to, XTEN (Schellenberger et al., Nat. Biotechnol. 27:1186-1192, 2009) and Albu tag (Trussel et al., Bioconjug Chem. 20:2286-2292, 2009).

In some embodiments, aPD-1 antibody or fragment thereof disclosed herein is conjugated to a chemically functional moiety. Typically, the moiety is a label capable of producing a detectable signal. These conjugated antibodies or fragments thereof are useful, for example, in detection systems such as quantitation of tumor burden, and imaging of metastatic foci and tumor imaging. Such labels are known in the art and include, but are not limited to, radioisotopes, enzymes, fluorescent compounds, chemiluminescent compounds, bioluminescent compounds substrate cofactors and inhibitors. See, for examples of patents describing the use of such labels, U.S. Pat. Nos. 3,817,837; 3,850,752; 3,939,350; 3,996,345; 4,277,437; 4,275,149; and 4,366,241. The moieties can be covalently linked to antibody or fragment thereof as described herein, recombinantly linked, or conjugated to an antibody or fragment thereof through a secondary reagent, such as a second antibody, protein A, or a biotin-avidin complex.

Other functional moieties include signal peptides, agents that enhance or reduce immunologic reactivity, agents that facilitate coupling to a solid support, vaccine carriers, bioresponse modifiers, paramagnetic labels and drugs. A signal peptide is a short amino acid sequence that directs a newly synthesized protein through a cellular membrane, usually the endoplasmic reticulum in eukaryotic cells, and either the inner membrane or both inner and outer membranes of bacteria. Signal peptides are typically at the N-terminal portion of a polypeptide and are typically removed enzymatically between biosynthesis and secretion of the polypeptide from the cell. Such a peptide can be incorporated into the subject antibody or fragment thereof to allow secretion of the synthesized molecules.

Agents that enhance immunologic reactivity include, but are not limited to, bacterial superantigens. Agents that facilitate coupling to a solid support include, but are not limited to, biotin or avidin. Immunogen carriers include, but are not limited to, any physiologically acceptable buffers. Bioresponse modifiers include cytokines, particularly tumor necrosis factor (TNF), interleukin-2, interleukin-4, granulocyte macrophage colony stimulating factor and gamma.-interferons.

Agents that reduce immunologic reactivity include, but are not limited to anti-inflammatory agents and immunosuppressants. Anti-inflammatory agents include non-steroidal anti-inflammatory drugs (NSAIDs) and corticosteroids. NSAIDs include but are not limited to, salicylates, such as acetylsalicylic acid; diflunisal, salicylic acid, and salsalate; propionic acid derivatives, such as ibuprofen; naproxen; dexibuprofen, dexketoprofen, flurbiprofen, oxaprozin, fenoprofen, loxoprofen, and ketoprofen; acetic acid derivatives, such as indomethacin, diclofenac, tolmetin, aceclofenac, sulindac, nabumetone, etodolac, and ketorolac; enolic acid derivatives, such as piroxicam, lornoxicam, meloxicam, isoxicam, tenoxicam, phenylbutazone, and droxicam; anthranilic acid derivatives, such as mefenamic acid, flufenamic acid, meclofenamic acid, and tolfenamic acid; selective COX-2 inhibitors, such as celecoxib, lumiracoxib, rofecoxib, etoricoxib, valdecoxib, firocoxib, and parecoxib; sulfonanilides, such as nimesulide; and others such as clonixin, and licofelone. Corticosteroids include but are not limited to, cortisone, dexamethasone, hydrocortisone, methylprednisolone, prednisone, and prednisolone. The immunosuppressants include but are not limited to hydroxychloroquine, sulfasalazine, leflunomide, etanercept, infliximab, adalimumab, D-penicillamine, oral gold compound, injectable gold compound (intramuscular injection), minocycline, sodium gold thiomalate, auranofin, D-penicillamine, lobenzarit, bucillamine, actarit, cyclophosphamide, azathioprine, methotrexate, mizoribine, cyclosporine, and tacrolimus.

Suitable drug moieties include antineoplastic agents. Non-limiting examples are radioisotopes, vinca alkaloids such as the vinblastine, vincristine and vindesine sulfates, adriamycin, bleomycin sulfate, carboplatin, cisplatin, cyclophosphamide, cytarabine, dacarbazine, dactinomycin, duanorubicin hydrochloride, doxorubicin hydrochloride, etoposide, fluorouracil, lomustine, mechlororethamine hydrochloride, melphalan, mercaptopurine, methotrexate, mitomycin, mitotane, pentostatin, pipobroman, procarbaze hydrochloride, streptozotocin, taxol, thioguanine, and uracil mustard.

Immunotoxins, including single chain molecules, can be produced by recombinant means. A variety of immunotoxins are available, and methods can be found, for example, in Monoclonal Antibody-toxin Conjugates: Aiming the Magic Bullet, Thorpe et al. (1982) Monoclonal Antibodies in Clinical Medicine, Academic Press, pp. 168-190; Vitatta (1987) Science 238:1098-1104; and Winter and Milstein (1991) Nature 349:293-299. Suitable toxins include, but are not limited to, ricin, radionuclides, pokeweed antiviral protein, Pseudomonas exotoxin A, diphtheria toxin, ricin A chain, fungal toxins such as restrictocin and phospholipase enzymes. See, generally, “Chimeric Toxins,” Olsnes and Pihl, Pharmac. Ther. 15:355-381 (1981); and “Monoclonal Antibodies for Cancer Detection and Therapy,” eds. Baldwin and Byers, pp. 159-179, 224-266, Academic Press (1985).

The chemically functional moieties can be made recombinantly for instance by creating a fusion gene encoding the antibody and the functional moiety. Alternatively, the antibody or fragment thereof can be chemically bonded to the moiety by any of a variety of well-established chemical procedures. For example, when the moiety is a protein, a variety of coupling agents may be used such as N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCl), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). The linker may be a “cleavable linker” facilitating release of the cytotoxic drug in the cell. For example, an acid-labile linker, peptidase-sensitive linker, dimethyl linker, or disulfide-containing linker (Chari et al. Cancer Research, 52: 127-131 (1992)) may be used. The moieties may be covalently linked, or conjugated, through a secondary reagent, such as a second antibody, protein A, or a biotin-avidin complex. For examples of paramagnetic moieties and the conjugation thereof to antibodies, see, e.g., Miltenyi et al. (1990) Cytometry 11:231-238.

In some embodiments, a PD-1 antibody or fragment thereof disclosed herein is a bispecific antibody. Bispecific antibodies are antibodies that have binding specificities for at least two different epitopes. A bispecific antibody as described herein may be a bispecific antibody that recognizes different epitopes on PD-1, or a bispecific antibody in which one of the antigen-binding sites recognizes PD-1 and the other antigen-binding site recognizes an antigen other than PD-1.

### Nucleic Acid Molecules

In some embodiments, the antibody described herein is encoded by one or more nucleic acid molecules. In one case, the antibody is encoded by a single nucleic acid molecule. In other cases, the antibody is encoded by two or more nucleic acid molecules. For example, as the antigen binding site is formed by the coming together of a heavy chain variable polypeptide region and a light chain variable polypeptide region, the two variable (heavy and light) polypeptide regions are encoded by separate nucleic acid molecules. Alternatively, for example, in the case of an ScFv, they are encoded by the same nucleic acid molecule.

According to some aspects of the disclosure there is provided one or more nucleic acid molecules that encode an antibody or antigen-binding fragment thereof in accordance with some embodiments of the present disclosure.

From the primary amino acid sequence of the polypeptide(s) encoding the antibody provided herein, the person of skill in the art is able to determine suitable nucleotide sequence(s) that encodes the polypeptide(s) and, if desired, one that is codon-optimized (e.g., see Mauro and Chappell. Trends Mol Med. 20(11):604-613, 2014).

According to some aspects of the disclosure there is provided an isolated nucleic acid comprising a nucleotide sequence that encodes a heavy chain variable region polypeptide or a light chain variable region polypeptide of the disclosure. A heavy chain variable polypeptide or a light chain variable polypeptide of the disclosure refers to the individual polypeptide chains that include amino acids that make up part of the antigen-binding site. In some cases, the polypeptides also comprise other domains such as constant domains, hinge regions, and an Fc region, such as one comprising one or more Fc receptor binding sites.

According to some aspects of the disclosure there is provided an isolated nucleic acid which comprises one or more nucleotide sequence encoding polypeptides capable of forming an antibody or antigen-binding fragment of the disclosure. In particular embodiments, the polypeptides may also comprise other domains such as constant domains, hinge regions, and an Fc region, such as one comprising one or more Fc receptor binding sites.

In one case, the nucleic acid molecule encodes just the polypeptide sequence that comprises the VL domain of the antibody or fragment thereof. In some cases, the nucleic acid molecule encodes just the polypeptide sequence that comprises the VH domain of the antibody or fragment thereof. In other cases, the nucleic acid molecule encodes both VH and VL domain containing polypeptide sequences capable of forming the antibody or antibody fragment thereof of the disclosure.

The nucleic acid molecule(s) that encode the antibody or antigen-binding fragment thereof of the disclosure may be, or may be part of, a vector (such as a plasmid vector, cosmid vector or viral vector, or an artificial chromosome) that may comprise other functional regions (elements) such as one or more promoters, one or more origins or replication, one or more selectable marker(s), and one or more other elements typically found in expression vectors. The cloning and expression of nucleic acids that encode proteins, including antibodies, is well established and well within the skill of the person in the art.

### Vectors

According to some aspects of the disclosure there is provided a vector comprising the nucleic acid according to some embodiments of the disclosure. In particular embodiments, the vector is a plasmid vector, cosmid vector, viral vector, or an artificial chromosome.

The nucleic acids of the present disclosure, including vector nucleic acids that comprise nucleotide sequences that encode the polypeptides capable of forming an antibody of the disclosure or an antigen-binding fragments thereof, may be in purified/isolated form.

Isolated/purified nucleic acids that encode an antibody or antigen-binding fragment thereof of the disclosure will be free or substantially free of material with which they are naturally associated, such as other proteins or nucleic acids with which they are found in their natural environment, or the environment in which they are prepared (e.g., cell culture) when such preparation is by recombinant DNA technology practised in vitro or in vivo.

In some embodiments, the nucleic acids of the disclosure are greater than 80%, such as greater than 90%, greater than 95%, greater than 97% and greater than 99% pure.

Thus, according to some aspects of the disclosure there is provided a vector comprising a nucleic acid or nucleotide sequence that encodes a heavy chain variable polypeptide or a light chain variable polypeptide of the disclosure. In a particular embodiment, the vector comprises nucleic acid that encodes both the heavy and light chain variable regions. In some embodiments, the polypeptides comprise other domains such as constant domains, hinge regions, and an Fc region, such as one comprising one or more Fc receptor binding sites.

In some embodiments, the nucleic acid and/or vector of the disclosure is introduced into a host cell. For eukaryotic cells, for example, suitable techniques include calcium phosphate transfection, DEAE-Dextran, electroporation, liposome- mediated transfection and transduction using retrovirus or other virus, e.g., vaccinia or, for insect cells, baculovirus. In one aspect, introducing nucleic acid in the host cell, in particular a eukaryotic cell, uses a viral or a plasmid-based system. In some cases, the plasmid system is maintained episomally. In other cases, the plasmid system is incorporated into the host cell or into an artificial chromosome. In a particular embodiment, the incorporation is by random integration of one or more copies at single or multiple loci. In some embodiments, the incorporation is by targeted integration of one or more copies at single or multiple loci. For bacterial cells, suitable techniques include, for example, calcium chloride transformation, electroporation and transfection using bacteriophage.

In one embodiment, the nucleic acid of the disclosure is integrated into the genome (e.g., chromosome) of the host cell. In a particular embodiment, integration is promoted by inclusion of sequences that promote recombination with the genome, in accordance with standard techniques.

### Host Cells

A further aspect of the present disclosure provides a host cell containing nucleic acid as disclosed herein. In some embodiments, such a host cell is in vitro. In some embodiments, such a host cell is in culture.

In some cases, the host cell is from any species, such as a bacterium or yeast. In other cases, the host cell is a mammalian cell such as a human cell or rodent cell, for example a HEK293T cell or CHO-K1 cell.

Thus, according to some aspects of the disclosure there is provided a host cell comprising the nucleic acid sequence or the vector according to some embodiments of the present disclosure.

In some cases, the host cell is treated so as to cause or allow expression of the protein of the disclosure from the nucleic acid, e.g., by culturing host cells under conditions for expression of the encoding nucleic acid. In some embodiments, the purification of the expressed product is achieved by methods known to one of skill in the art.

In some embodiments, the nucleic acids of the disclosure, including vector nucleic acids that comprise nucleotide sequences that encode the polypeptides for the antibodies of the disclosure or antigen-binding fragments thereof, is present in an isolated host cell. In some cases, the host cell is part of a clonal population of host cells. As used herein, reference to a host cell also encompasses a clonal population of the cell. A clonal population is one that has been grown from a single parent host cell. In some cases, the host cell is from any suitable organism. In some cases, the host cell is, for example, bacterial, fungal or mammalian cells.

In some embodiments, the host cell assists in amplifying the vector nucleic acid (such as with a plasmid). In a particular embodiment, the host cell serves as the biological factory to express the polypeptide(s) of the disclosure that form the PD-1 antibody or fragment thereof described herein. In one case, a suitable host for amplifying the vector nucleic acid is a bacterial or fungal cell, such as an Escherichia coli cell or Saccharomyces cerevisiae cell. In other cases, a suitable host for expressing the proteins of the disclosure (i.e., the polypeptides making up the PD-1 antibody or fragment thereof of the disclosure) is a mammalian cell such as a HEK293T or CHO-K1 cell. In a particular embodiment, the host cell is a mammalian cell, such as a HEK293T or CHO-K1 cell.

A variety of host-expression vector systems is suitable to express a PD-1 antibody or fragment thereof as described herein. Different host cells have characteristic and specific mechanisms for the post-translational processing and modification of proteins and gene products. Appropriate cell lines or host systems is chosen to ensure the correct modification and processing of the protein of the disclosure. In some embodiments, eukaryotic host cells which possess the cellular machinery for proper processing of the primary transcript, glycosylation, and phosphorylation of the gene product is used. Such mammalian host cells include but are not limited to CHO, HEK, VERY, BHK, Hela, COS, MDCK, 293, 3T3, W138, BT483, Hs578T, HTB2, BT2O and T47D, NS0, CRL7O3O and HsS78Bst cells.

### Antibody Manufacturing

The PD-1 antibody or fragment thereof disclosed herein can be produced as a recombinant antibody by cloning DNA encoding the subject antibody or peptide from hybridomas or B cells or any form of antibody and/or antibody fragment libraries, integrating the clone into a suitable vector, and transducing the vector into host cells (for example, P. J. Delves, Antibody Production: Essential Techniques, 1997 WILEY, P. Shepherd and C. Dean Monoclonal Antibodies, 2000 OXFORD UNIVERSITY PRESS, Vandamme A. M. et al., Eur. J. Biochem. 192:767-775 (1990)). Thus, in one aspect, provided herein is an isolated polynucleotide encoding an antibody or fragment thereof of the present disclosure.

Nucleotide sequences corresponding to various regions of L or H chains of an existing antibody can be readily obtained and sequenced using convention techniques including but not limited to hybridization, PCR, and DNA sequencing. Hybridoma cells that produce monoclonal antibodies serve as one source of antibody nucleotide sequences. A vast number of hybridoma cells producing an array of monoclonal antibodies may be obtained from public or private repositories. The largest depository agent is American Type Culture Collection, which offers a diverse collection of well-characterized hybridoma cell lines. Alternatively, antibody nucleotides can be obtained from immunized or non-immunized rodents or humans, and from organs such as spleen and peripheral blood lymphocytes. Specific techniques applicable for extracting and synthesizing antibody nucleotides are described in Orlandi et al. (1989) Proc. Natl. Acad. Sci. U.S.A 86: 3833-3837, Larrick et al. 1989) biochem. Biophys. Res. Commun. 160: 1250-1255; Sastry et al. (1989) Proc. Natl. Acad. Sci., U.S.A. 86: 5728-5732; and U.S. Pat. No. 5,969,108.

The PD-1 antibody nucleotide sequences may also be modified, for example, by substituting the coding sequence for human heavy and light chain constant regions in place of the homologous non-human sequences. In that manner, chimeric antibodies are prepared that retain the binding specificity of the original antibody.

Additionally, polynucleotides encoding the heavy and/or light chains of the PD-1antibody or a functional fragment thereof can be subjected to codon optimization to achieve optimized expression of a subject antibody or functional fragment thereof in a desired host cell. For example, in one method of codon optimization, a native codon is substituted by the most frequent codon from a reference set of genes, wherein the rate of codon translation for each amino acid is designed to be high. Additional exemplary methods for generating codon optimized polynucleotides for expression of a desired protein, which can be applied to the heavy and/or light chains of the PD-1 antibody or a functional fragment thereof, are described in Kanaya et al., Gene, 238:143-155 (1999), Wang et al., Mol. Biol. Evol., 18(5):792-800 (2001), U.S. Pat. No. 5,795,737, U.S. Publication 2008/0076161 and WO 2008/000632.

Polynucleotides of the PD-1 antibody of the present disclosure include those coding for functional equivalents and fragments thereof of the exemplified polypeptides. Functional equivalents may be polypeptides having conservative amino acid substitutions, analogs including fusions, and mutants.

Due to the degeneracy of the genetic code, there can be considerable variation in nucleotides of the L and H sequences, as well as the heterodimerization sequences suitable for construction of the polynucleotide and vectors of the present disclosure. These variation are encompassed by the present disclosure.

Where desired, the recombinant polynucleotides can comprise heterologous sequences that facilitate detection of the expression and purification of the gene product. Examples of such sequences include those encoding reporter proteins such as β-galactosidase, β-lactamase, chloramphenicol acetyltransferase (CAT), luciferase, green fluorescent protein (GFP) and their derivatives. Other heterologous sequences that facilitate purification may code for epitopes such as Myc, HA (derived from influenza virus hemagglutinin), His-6, FLAG, or the Fc portion of immunoglobulin, glutathione S-transferase (GST), and maltose-binding protein (MBP).

The polynucleotides can be conjugated to a variety of chemically functional moieties as described above. Commonly employed moieties include labels capable of producing a detectable signal, signal peptides, agents that enhance or reduce immunologic reactivity, agents that facilitate coupling to a solid support, vaccine carriers, bioresponse modifiers, paramagnetic labels and drugs. The moieties can be covalently linked to a polynucleotide recombinantly or by other means known in the art.

The polynucleotides can comprise additional sequences, such as additional encoding sequences within the same transcription unit, controlling elements such as promoters, ribosome binding sites, and polyadenylation sites, additional transcription units under control of the same or a different promoter, sequences that permit cloning, expression, and transformation of a host cell, and any such construct as may be desirable in accordance with any of the various embodiments described herein.

The polynucleotides can be obtained using chemical synthesis, recombinant cloning methods, PCR, or any combination thereof. One of skill in the art can use the sequence data provided herein to obtain a desired polynucleotide by employing a DNA synthesizer or ordering from a commercial service.

Polynucleotides comprising a desired sequence can be inserted into a suitable vector which in turn can be introduced into a suitable host cell for replication, amplification and expression. Accordingly, in one aspect, provided herein are a variety of vectors comprising one or more of the polynucleotides of the present disclosure. Also provided is a selectable library of expression vectors comprising at least one vector encoding the subject antibody.

In some aspects, provided herein is a polynucleotide sequence encoding at least a portion of the heavy chain or light chain of the antibody or fragment thereof disclosed herein. In some aspects, provided herein is a vector comprising the polynucleotide sequence disclosed herein.

Vectors of the present disclosure are generally categorized into cloning and expression vectors. Cloning vectors are useful for obtaining replicate copies of the polynucleotides they contain, or as a means of storing the polynucleotides in a depository for future recovery. Expression vectors (and host cells containing these expression vectors) can be used to obtain polypeptides produced from the polynucleotides they contain. Suitable cloning and expression vectors include any known in the art, e.g., those for use in bacterial, mammalian, yeast, insect and phage display expression systems.

Suitable cloning vectors can be constructed according to standard techniques, or selected from a large number of cloning vectors available in the art. While the cloning vector selected may vary according to the host cell intended to be used, useful cloning vectors will generally have the ability to self-replicate, may possess a single target for a particular restriction endonuclease, or may carry marker genes. Suitable examples include plasmids and bacterial viruses, e.g., pBR322, pMB9, ColE1, pCR1, RP4, pUC18, mp18, mp19, phage DNAs (including filamentous and non-filamentous phage DNAs), and shuttle vectors such as pSA3 and pAT28. These and other cloning vectors are available from commercial vendors such as Clontech, BiORad, Stratagene, and Invitrogen.

Expression vectors containing these nucleic acids are useful to obtain host vector systems to produce proteins and polypeptides. Typically, these expression vectors are replicable in the host organisms either as episomes or as an integral part of the chromosomal DNA. Suitable expression vectors include plasmids, viral vectors, including phagemids, adenoviruses, adeno-associated viruses, retroviruses, cosmids, etc. A number of expression vectors suitable for expression in eukaryotic cells including yeast, avian, and mammalian cells are available. One example of an expression vector is pcDNA3 (Invitrogen, San Diego, Calif.), in which transcription is driven by the cytomegalovirus (CMV) early promoter/enhancer. Two types of particularly useful expression vectors for expressing the subject antibody as described herein are the phage display vector and bacterial display vector.

The vectors of the present disclosure can comprise transcriptional or translational control sequences required for expressing the encoded antibody. Suitable transcription or translational control sequences include but are not limited to replication origin, promoter, enhancer, repressor binding regions, transcription initiation sites, ribosome binding sites, translation initiation sites, and termination sites for transcription and translation.

The expression vector can be transferred to a host cell and the transfected cells are then cultured to produce a subject antibody or functional fragment thereof. Thus, in one aspect, provided herein are host cells containing a polynucleotide encoding a subject antibody or functional fragment thereof operably linked to a heterologous promoter. The host cell can be co-transfected with two expression vectors, the first vector encoding a heavy chain derived polypeptide and the second vector encoding a light chain derived polypeptide. The two vectors can contain identical selectable markers which enable equal expression of heavy and light chain polypeptides. Alternatively, a single vector can be used which encodes, and is capable of expressing, both heavy and light chain polypeptides. In such situations, the light chain can be placed before the heavy chain to avoid an excess of toxic free heavy chain (Proudfoot, 1986, Nature 322:52; and Kohler, 1980, Proc. Natl. Acad. Sci. USA 77:2197-2199).

A variety of host-expression vector systems can be utilized to express the subject antibody or functional fragment thereof (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express a subject antibody molecule in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NSO, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5 K promoter). For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In some embodiments, antibodies or fragments thereof are produced in CHO cells.

For bacterial systems, a number of expression vectors may be advantageously selected depending upon the use intended for the antibody molecule being expressed. For example, when a large quantity of such an antibody or fragment thereof is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) can be used as a vector to express foreign genes. The virus grows in Spodoptera frugiperda cells. The antibody or functional fragment coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be used for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

For plant cells, a variety of vector delivery techniques is available in the art. The host cells may be in the form of whole plants, isolated cells or protoplasts. Illustrative procedures for introducing vectors into plant cells include Agrobacterium-mediated plant transformation, protoplast transformation, gene transfer into pollen, injection into reproductive organs and injection into immature embryos. As is evident to one skilled in the art, each of these methods has distinct advantages and disadvantages. Thus, one particular method of introducing vectors into a particular plant species may not necessarily be the most effective for another plant species.

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the antibody or functional fragment. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERY, BHK, Hela, COS, MDCK, 293, 3T3, W138, BT483, Hs578T, HTB2, BT2O and T47D, NSO (a murine myeloma cell line that does not endogenously produce any immunoglobulin chains), CRL7O3O and HsS78Bst cells.

For long-term, high-yield production of recombinant proteins, stable expression is preferred. For example, cell lines which stably express an antibody or functional fragment thereof can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule.

A number of selection systems can be used, including but not limited to, systems using the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes in tk-, hgprt- or aprt-cells, respectively. Also, antimetabolite resistance can be used as the basis of selection for the following genes: dhfr, which confers resistance to methotrexate (Wigler et al., 1980, Proc. Natl. Acad. Sci. USA. 77(6):3567-70; O’Hare et al., 1981, Proc. Natl. Acad. Sci. USA 78:1527); glutamine synthetase (GS), which is an enzyme responsible for the biosynthesis of glutamine using glutamate and ammonia (Bebbington et al., 1992, Biuotechnology 10:169); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-215); and hygro, which confers resistance to hygromycin (Santerre et al., 1984, Gene 30:147). Recombinant DNA technology methods can be applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties. The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing an antibody or functional fragment thereof is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

Once an antibody molecule has been produced by recombinant expression, it can be purified by any suitable method for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the subject antibodies or functional fragments thereof can be fused to heterologous polypeptide sequences provided herein or otherwise known in the art to facilitate purification. For example, a subject antibody or functional fragment thereof can be purified through recombinantly adding a poly-histidine tag (His-tag), FLAG-tag, hemagglutinin tag (HA-tag) or myc-tag among others that are commercially available and utilizing suitable purification methods.

### Method of Treatment

In another aspect, provided herein are methods of using the antibody or a functional fragment thereof disclosed herein to suppress an immune cell in vitro, ex vivo, or in vivo. In some cases, the immune cell is a T cell, B cell, a macrophage, or any other immune cell. In some cases, the immune cell is an effector T cell. In some cases, the immune cells is an antigen-specific T cell. In some cases, the method disclosed herein is applicable to treat a subject in need thereof. In some cases, the method comprises administering the antibody or fragment thereof disclosed herein to a subject in need thereof. In some cases, the method comprises suppressing an immune cell in vitro and transferring the immune cell to a subject in need thereof.

In another aspect, the antibodies of the invention can be used as a targeting agent for delivery of another therapeutic or a cytotoxic agent (e.g., a toxin) to a cell expressing PD-1. The method includes administering an anti-PD-1 antibody coupled to a therapeutic or a cytotoxic agent or under conditions that allow binding of the antibody to PD-1 expressed on the cell surface.

In another aspect, provided herein are methods of using the antibody or a functional fragment thereof disclosed herein to treat diseases or conditions in a subject in need thereof. In some cases, the diseases or conditions the subject method is applicable to are associated with PD-1 or PD-L1 signaling. In some cases, the diseases or conditions are inflammatory disorder, autoimmune disorders, and/or associated with excess or undesirable immune response. In some embodiments, the present disclosure provides a method of treating an inflammatory disorder in a mammal, e.g., a human, in need thereof, comprising administering to the mammal a therapeutically effective amount of an antibody of the present disclosure. In some cases, the inflammatory disorder is multiple sclerosis. In other cases, the inflammatory disorder is an autoimmune disease. In some cases, the disease or condition is adult-onset Still’s disease; alcoholic hepatitis, alcoholic steatohepatitis, alcoholic liver disease, asthma, including allergen-induced asthma, bullous pemphigoid (BP) asthma, non-allergen induced asthma, allergies and allergic conditions such as allergic bronchopulmonary aspergillosis, allergic conjunctivitis, allergic encephalomyelitis, and allergic neuritis, food allergies, allograft rejection, alcoholic steatohepatitis (ASH), ANCA vasculitis, anti-glomerular basement membrane disease (Anti-GBM), antiphospholipid syndrome, aphthous stomatitis, appendicitis, arthritis, autoimmune diseases, atrophic thyroiditis, autoimmune hemolytic anemia (immune pancytopenia, paroxysmal nocturnal hemoglobinuria), autoimmune polyendocrinopathies, autoimmune thrombocytopenia (idiopathic thrombocytopenic purpura, immune-mediated thrombocytopenia), autoimmune hepatitis, pernicious anemia (Addison’s disease), and autoimmune thyroid disorders, autoinflammatory diseases, autosomal dominant polyscystic kidney disease (ADPKD), ankylosing spondylitis (AS), acute respiratory distress syndrome (ARDS), Bechet’s disease or syndrome, bee sting-induced inflammation, Blau syndrome, bursitis, Barrett’s esophagus, bleomycin induced pulmonary fibrosis, bronchiolitis obliterans; cardiac hypertrophy, gluten-sensitive enteropathy (Celiac disease), chemical irritant-induced inflammation, chorioretinitis, chronic atypical neutrophilic dermatosis with lipodystrophy and elevated temperature (CANDLE) syndrome, chronic obstructive pulmonary disease (COPD), chronic pancreatitis, chronic prostatitis, chronic recurrent multifocal osteomyelitis, cicatricial alopecia, colitis, complex regional pain syndrome, chronic intrahepatic or extrahepatic cholestatic disease, conjunctivitis, connective tissue disease, Connective tissue disease-associated interstitial lung disease (CTD-ILD), corneal ulcer, cryopyrin-associated periodic syndromes, cutaneous lupus erythematosus (CLE), cystic fibrosis, deficiency of the interleukin-1 receptor antagonist (DIRA), deficiency of IL36R antagonist (DITRA), dermatitis, diabetic kidney disease (DKD) (diabetic nephropathy), diverticulitis, discoid lupus erythematosus, drug induced delayed type cutaneous allergic reactions, encephalitis, esophagitis, eosinophilic gastrointestinal disorders (EGIDs), such as eosinophilic esophagitis (EoE), eosinophilic gastroenteritis, eosinophilic colitis; familial cold urticarial, familial Mediterranean fever, fistulizing Crohn’s disease, giant cell arteritis, glomerulonephritis, gout, gouty arthritis, graft-versus-host disease (GVHD), granulomatous hepatitis, Guillain-Barre syndrome (GBS), Graves’ disease, Hashimoto’s thyroiditis; Henoch-Schönlein purpura, hidradenitis suppurativa (HS), hyaline membrane disease, hyperactive inflammatory response, hypereosinophilic syndrome (HES), hyperimmunoglobulinemia D with recurrent fever (HIDS), hypersensitivity pneumonitis (HP), immunoglobulin (IgA) nephropathies, IgG4-related disease, immune complex nephritis, immune thrombocytopenic purpura (ITP), inflammation, inflammation of the CNS, inflammatory bowel disease (IBD), inflammatory disease of the respiratory tract (upper or lower) such as inflammatory lung disease, bronchitis, sinusitis, inflammatory ischemic event such as stroke or cardiac arrest, inflammatory liver disease, inflammatory myopathy, inflammatory neuropathy, inflammatory pain, insect bite-induced inflammation, interstitial cystitis, iritis, irritant-induced inflammation, juvenile arthritis, juvenile rheumatoid arthritis, keratitis, kidney transplant rejection, kidney disease, kidney fibrosis, kidney insufficiency, leukocyte adhesion deficiency, Loeffler’s syndrome, lupus, lupus nephritis (LN), liver fibrosis, liver steatosis; liver ischemia; lipid and lipoprotein disorders; mast cell activation syndrome, mastocytosis, meningitis, microscopic colitis, mixed connective tissue disease, morphea or morphea variants, Muckle-Wells syndrome (urticaria deafness amyloidosis), mucositis, myelitis, myocarditis, myositis, necrotizing enterocolitis, neonatal onset multisystem inflammatory disease (NOMID), nasal polyps, neovascular glaucoma, neuritis, non-alcoholic fatty liver disease (NAFLD), non-alcoholic steatohepatitis (NASH), non-radiographic axial spondyloarthritis (nr-AxSpA), non-cystic fibrosis bronchiectasis (non-CFB), obstructive or chronic inflammatory disorders of the liver; ocular allergy, optic neuritis, organ transplant rejection, osteoarthritis (OA), otitis, pancreatitis, pancolitis, pelvic inflammatory disease, pemphigus vulgaris (PV), bullous pemphigoid (BP), pericarditis, periodontitis, PFAPA (periodic fever, aphthous stomatitis, pharyngitis, adenitis), plant irritant-induced inflammation, pneumocystis infection, pneumonia, pneumonitis, poison ivy/ urushiol oil-induced inflammation, polyarteritis nodosa, polychondritis, polycystic kidney disease (PCKD), polymyalgia rheumatic, polymyositis, pouchitis, proctitis, proctosignmoiditis, psoriatic arthritis (PsA), pulmonary arterial hypertension (PAH), pulmonary fibrosis, pyogenic sterile arthritis, pruritus, reperfusion injury and transplant rejection, primary biliary cirrhosis (PBC), primary sclerosing cholangitis (PSC), Raynaud’s syndrome, Reiter’s disease, reactive arthritis, renal graft rejection, reperfusion injury, rheumatic carditis, rheumatic diseases, rheumatic fever, rheumatoid arthritis (RA), rhinitis, rhinitis psoriasis, sarcoidosis, Schnitzler syndrome, scleritis, sclerosis, such as systemic sclerosis (SSc), seborrhea, sepsis, septic shock, Sjogren’s syndrome, inflammatory skin diseases or conditions, such as acne, alopecia areata, atopic dermatitis, rosacea, eczema, dermatitis, dermatitis endotoxemia, dermatomyositis, stasis dermatitis, Stevens-Johnson syndrome (SJS), skin irritation, skin rash, skin sensitization (contact dermatitis or allergic contact dermatitis), scleroderma, psoriasis, psoriasis vulgaris, psoriatic arthritis, ; spinal stenosis, spondyloarthropathies, synovial inflammation, systemic inflammatory response syndrome (SIRS), systemic lupus erythematosus (SLE), systemic mast cell disease (SMCD), systemic vasculitis, systemic-onset juvenile idiopathic arthritis, temporal arteritis, tendinitis, tenosynovitis, thyroditis, transplantation rejection, tubulointerstitial nephritis, tubular disfunction, Takayasu arteritis, toxic epidermal necrolysis,urticaria, uterine fibroids, uveitis, uveoretinitis, vasculitis, vasculitis (NHLBI), vitiligo, or Wegener’s granulomatosis. In some cases, the disease or condition is acne, acid-induced lung injury, Addison’s disease, adrenal hyperplasia, adrenocortical insufficiency, age-related macular degeneration, aging, alcoholic liver disease, Alzheimer’s disease, angina pectoris, angiofibroma, anhidrotic ectodermal dysplasia, ascites, aspergillosis, atherosclerosis, atherosclerotic plaques, amyloidosis, amyotrophic lateral sclerosis (ALS), angioedema, acute myocardial infarction; antigen-antibody complex mediated diseases, alpha-1-antitrypsin deficiency; back pain, Bacillus anthracis infection, Bell’s palsy, berylliosis, bone pain, burns, bullous pemphigoid, cancer, carpal tunnel syndrome, Castleman’s disease, catabolic disorders, cataracts, cerebral aneurysm, complications of organ transplantation, corneal graft neovascularization, cryptococcosis, a non-malignant hyperproliferative disorder; a malignant hyperproliferative disorder; hepatocellular carcinoma; colon adenoma; polyposis; colon adenocarcinoma; breast cancer; pancreatic adenocarcinoma, chronic heart failure, chronic lung disease of prematurity, cardiometabolic syndrome, cardiovascular disease, cutaneous T cell lymphoma, diabetic macular edema, dyslipidemia; endometriosis, endotoxemia, eosinophilic GI disease (EGID), eosinophilic esophagitis (EoE), eosinophilic pneumonias, epicondylitis, epidermolysis bullosa, erythema multiforme, erythroblastopenia, familial amyloidotic polyneuropathy, fetal growth retardation, fibromyalgia, glaucoma, glioblastoma, glomerular disease, gut diseases, growth plate injuries, hair loss, herpes zoster and simplex, hypoplastic and other anemias, head injury, hepatitis A, B, C, D, and E, herpes; headache, hearing loss, heart disease, hemangioma, hemophilic joints, hereditary periodic fever syndrome, heritable disorders of connective tissue, Hodgkin’s disease, Huntington’s disease, hyperammonemia, hypercalcemia, hypercholesterolemia, hemolytic anemia, hepatitis, hip replacement, hypertropic bone formation, hypersensitivity pneumonia, hereditary fructose intolerance, hypertension, hyperuricemia, idiopathic demyelinating polyneuropathy, infectious diseases including viral diseases such as AIDS (HIV infection), ichthyosis, incontinentia pigmenti (IP, Bloch-Siemens syndrome), idiopathic thrombocytopenic purpura, infectious mononucleosis, ischemia/reperfusion, insulin resistance, joint replacement, kidney injury caused by parasitic infections, leptospirosis, lichen sclerosus (LS), lichen planus, Lambert-Eaton myasthenic syndrome, Lyme disease, liver failure, including acute liver failure, muscle wasting, muscular dystrophy, Marfan syndrome (MFS), meningioma, mesothelioma, multiple organ injury syndrome, myasthenia gravis (MG), myelodysplastic syndrome, metabolic syndrome, multiple sclerosis, nephrotic syndrome, neuropathological diseases, nuclear factor-kappa B essential modulator (NEMO) deficiency syndrome, obesity, Osler-Weber syndrome, osteogenesis imperfecta, osteonecrosis, osteoporosis, pachyonychia congenita, Paget’s disease, Paget’s disease of bone, Parkinson’s disease, periodic fever, pertussis, primary pulmonary hypertension, pyoderma gangrenosum, pyogenic granuloma retrolental fibroplasias, peritoneal endometriosis, Prurigo nodularis, psychosocial stress diseases, pulmonary disease, pulmonary hypertension, respiratory distress syndrome, renal disease, retinal disease, retrolental fibroplasia, renal transplant rejection, renal protection against drugs inducing Fanconi’s syndrome, respiratory tract illness caused by respiratory syncytial virus, rhinosinusitis; radiation induced fibrosis, sarcoidosis, severe pain, sleep apnea, scoliosis, sickle cell anemia, sports injuries, sprains and strains, sunburn, spinal cord injury, Sézary syndrome, silica-induced disease (Silicosis), subarachnoid hemorrhage, tuberculosis, tumor necrosis factor (TNF) receptor associated periodic syndrome (TRAPS), thrombosis; traumatic brain injury, tissue transplant, complications from type 1 or type 2 diabetes, toxoplasmosis, thrombocytopenia, trachoma, vascular restenosis, ventilator induced lung injury; Whipple’s disease or 2,8-dihydroxyadenine nephropathy.

Examples of the diseases or conditions that the subject antibody can treat include but are not limited to acute disseminated encephalomyelitis (ADEM), Addison’s disease, allergy, alopecia areata, amyotrophic lateral sclerosis, ANCA vasculitis, ankylosing spondylitis, anti-phospholipid syndrome, asthma (including allergic asthma), atopic dermatitis, autoimmune haemolytic anaemia, autoimmune hepatitis, autoimmune pancreatitis, autoimmune polyendocrine syndrome, Behcet’s disease, bullous pemphigoid, cerebral malaria, chronic inflammatory demyelinating polyneuropathy, coeliac disease, Crohn’s disease, Cushing’s Syndrome, dermatomyositis, diabetes mellitus type 1, eosinophilic granulomatosis with polyangiitis, graft versus host disease, Graves’ disease, Guillain-Barre syndrome, Hashimoto’s thyroiditis, Hidradenitis Suppurativa, IgG4-related disease, inflammatory fibrosis (e.g., scleroderma, lung fibrosis, and cirrhosis), juvenile arthritis, Kawasaki disease, leukemia, lupus nephritis, lymphoma, lymphoproliferative disorders, multiple sclerosis, myasthenia gravis, myeloma, non-radiographic axial spondyloarthritis (nr-AxSpA), neuromyelitis optica, osteoarthritis, pemphigus, polymyositis, primary biliary cholangitis, primary sclerosing cholangitis, psoriasis, psoriatic arthritis, rheumatoid arthritis, sarcoidosis, Sjögren’s syndrome, systemic lupus erythematosus, systemic sclerosis, Takayasu’s arteritis, temporal arteritis, transplant rejection, transverse myelitis, ulcerative colitis, uveitis, vasculitis, vitiligo and Vogt-Koyanagi-Harada Disease. In some cases, the disease or condition comprises rheumatoid arthritis. In some cases, the disease or condition comprises multiple sclerosis.

In some cases a method of treating a disease or condition in a subject in need thereof, comprises administering to the subject a therapeutically effective amount of the agonist antibody disclosed herein, administering to the subject the pharmaceutical composition comprising a therapeutically effective amount of the antibody disclosed herein, or the immunoconjugate, and at least one pharmaceutically acceptable excipient, wherein the disease or condition further comprises infection, endotoxic shock associated with infection, arthritis, rheumatoid arthritis, psoriatic arthritis, systemic onset juvenile idiopathic arthritis (JIA), inflammatory bowel disease (IBD), systemic lupus erythematosus (SLE), systemic sclerosis, asthma, atopic dermatitis, pelvic inflammatory disease, Alzheimer’s Disease, Crohn’s disease, ulcerative colitis, irritable bowel syndrome, multiple sclerosis, ankylosing spondylitis, dermatomyositis, uveitis, Peyronie’s Disease, coeliac disease, gallbladder disease, Pilonidal disease, peritonitis, psoriasis, vasculitis, surgical adhesions, stroke, Type I Diabetes, lyme arthritis, meningoencephalitis, immune mediated inflammatory disorders of the central and peripheral nervous system, autoimmune disorders, pancreatitis, trauma from surgery, graft-versus-host disease, transplant rejection, heart disease, bone resorption, burns patients, myocardial infarction, Paget’s disease, osteoporosis, sepsis, liver/lung fibrosis, periodontitis, hypochlorhydia, solid tumors (renal cell carcinoma), liver cancer, multiple myeloma, prostatic cancer, bladder cancer, pancreatic cancer, neurological cancers, and B-cell malignancies (e.g., Casteleman’s disease, certain lymphomas, chronic lymphocytic leukemia, and multiple myeloma), lupus nephritis, and osteoarthritis. In some cases, the disease or condition comprises Sjogren’s syndrome. In some cases, the disease or condition comprises inflammatory bowel disease (IBD). In some cases, the disease or condition comprises systemic lupus erythematosus (SLE). In some cases, the disease or condition comprises lupus nephritis (LN). In some cases, the disease or condition comprises vasculitis, such as anti-neutrophil cytoplasmic antibody (ANCA) Associated (ANCA) vasculitis. In some cases, the disease or condition comprises graft-versus-host disease (GvHD). In some cases, the disease or condition comprises type 1 diabetes. In some cases, the disease or condition comprises Behcet’s syndrome. In some cases, the disease or condition comprises sepsis. In some cases, the disease or condition comprises osteoarthritis (OA). In some cases, the disease or condition comprises systemic sclerosis (SSc). In some cases, the disease or condition comprises dermatomyositis. In some cases, the disease or condition comprises psoriatic arthritis (PsA). In some cases, the disease or condition comprises IgG4-related disease. In some cases, the disease or condition comprises non-radiographic axial spondyloarthritis (nr-AxSpA). In some cases, the disease or condition comprises polymyositis. In some cases, the disease or condition comprises Takayasu arteritis.

In some embodiments, the present disclosure provides a method of treating cancer in a mammal in need thereof, comprising administering to the mammal a therapeutically effective amount of an antibody of the present disclosure. In some cases, the cancer is hepatocellular carcinoma. In other cases, the cancer is acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, esophageal, testicular, gynecological, thyroid, CNS, PNS, AIDS related (e.g., Lymphoma and Kaposi’s Sarcoma) or Viral-Induced cancer.

In some embodiments, the subject to be treated is a mammal, such as a human. In some embodiments, the subject to be treated is a human. In other cases, the mammal is a mouse, a rat, a cat, a dog, a rabbit, a pig, a sheep, a horse, a bovine, a goat, a gerbil, a hamster, a guinea pig, a monkey or any other mammal. Many such mammals may be subjects that are known to the art as preclinical models for certain diseases or disorders, including inflammatory diseases, solid tumors and/or other cancers (e.g., Talmadge et al., 2007 Am. J. Pathol. 170:793; Kerbel, 2003 Canc. Biol. Therap. 2(4 Suppl 1):S134; Man et al., 2007 Canc. Met. Rev. 26:737; Cespedes et al., 2006 Clin. TransL Oncol. 8:318).

In another aspect, the disclosure provides methods of using the PD-1 antibody of the present disclosure to treat diseases or conditions in a mammal in conjunction with a second agent. The second agent could be administered together with, before, or after the antibody. In some embodiments, the second agent is an agent that acts to relieve the symptoms of inflammatory conditions described herein. Anti-inflammatory agents include non-steroidal anti-inflammatory drugs (NSAIDs) and corticosteroids. NSAIDs include but are not limited to, salicylates, such as acetylsalicylic acid; diflunisal, salicylic acid, and salsalate; propionic acid derivatives, such as ibuprofen; naproxen; dexibuprofen, dexketoprofen, flurbiprofen, oxaprozin, fenoprofen, loxoprofen, and ketoprofen; acetic acid derivatives, such as indomethacin, diclofenac, tolmetin, aceclofenac, sulindac, nabumetone, etodolac, and ketorolac; enolic acid derivatives, such as piroxicam, lornoxicam, meloxicam, isoxicam, tenoxicam, phenylbutazone, and droxicam; anthranilic acid derivatives, such as mefenamic acid, flufenamic acid, meclofenamic acid, and tolfenamic acid; selective COX-2 inhibitors, such as celecoxib, lumiracoxib, rofecoxib, etoricoxib, valdecoxib, firocoxib, and parecoxib; sulfonanilides, such as nimesulide; and others such as clonixin, and licofelone. Corticosteroids include but are not limited to, cortisone, dexamethasone, hydrocortisone, methylprednisolone, prednisone, and prednisolone.

In some embodiments, the second agent is an immunosuppressant. The immunosuppressants that can be used in combination with the subject antibody include but are not limited to hydroxychloroquine, sulfasalazine, leflunomide, etanercept, infliximab, adalimumab, D-penicillamine, oral gold compound, injectable gold compound (intramuscular injection), minocycline, sodium gold thiomalate, auranofin, D-penicillamine, lobenzarit, bucillamine, actarit, cyclophosphamide, azathioprine, methotrexate, mizoribine, cyclosporine, and tacrolimus.

In some embodiments, the second agent is useful for the treatment and/or prophylaxis of rheumatoid arthritis. Non-limiting examples of such agents include disease-modifying antirheumatic drugs (DMARDS), such as hydroxychloroquine, sulfasalazine, methotrexate, and leflunomide; TNF inhibitors (e.g., etanercept, adalimumab, infliximab, golimumab, certolizumab pegol), T cell costimulatory inhibitor, (e.g., abatacept), IL-6 receptor inhibitors (e.g., tocilizumab, sarilumab), anti-CD20 antibody (e.g., rituximab); and JAK inhibitors (e.g., tofacitinib, baricitinib, upadacitinib); NSAIDs, such as ibuprofen, naproxen, and diclofenac; COX-2 inhibitor, such as celecoxib and etoricoxib; steroids and corticosteroids, such as prednisolone and cortisone; and biological agents known for treatment and/or prophylaxis of such conditions, including for example etanercept (e.g., ENBREL), infliximab (e.g., REMICADE), adalimumab (e.g., HUMIRA), anakinra (e.g., KINARET), abatacept (ORENCIA), rituximab (e.g., RITUXAN), certolizumab (e.g., CIMZIA), golimumab (e.g., SIMPONI), and tocilizumab (e.g., ACTEMRA). In some embodiments, a compound of the disclosure is administered with two additional thereapeutic agents useful for the treatment and/or prophylaxis of a rheumatological condition. In some embodiments, agents useful for the treatment and/or prophylaxis of a rheumatological condition include a compound of the disclosure and two additional therapeutic agents, such as methotrexate +leflunomide, methotrexate + sulfasalazine, methotrexate +cyclosporine, methotrexate + hydroxychloroquine and triple therapy treatments hydroxychloroquine + sulfasalazine + methotrexate, hydroxychloroquine + sulfasalazine + leflunomide.

In some embodiments, the second agent is useful for the treatment and/or prophylaxis of systemic lupus erythematosus (SLE) or lupus nephritis (LN). Non-limiting examples of such agents include immunosuppressive drugs that inhibit activity of the immune system and agents approved for treatment of SLE, such as hydroxychloroquine, steroids and corticosteroids (e.g., prednisone, methylprednisolone), belimumab, azathioprine, methotrexate, cyclophosphamide, mycophenolate and mycophenolate mofetil, cyclosporine, leflunomide, voclosporin, abatacept, anifrolumab, rituximab, NSAIDS, such as naproxen sodium and ibuprofen, antimalarial drugs, such as hydroxychloroquine, calcineurin inhibitors, and tacrolimus.

In some embodiments, the second agent is useful for the treatment of LN, such as prednisone + mycophenolic acid analogs, prednisone + mycophenolic acid sodium prednisone + cyclophosphamide, prednisone + tacrolimus, prednisone + voclosporin, prednisone + belimumab + mycophenolic acid analogs, prednisone + belimumab +cyclophosphamide, prednisone +rituximab.

In some embodiments, the second agent is useful for the treatment of LN, such as prednisone + mycophenolic acid analogs, prednisone + mycophenolic acid sodium, prednisone + Azathioprine, prednisone + Tacrolimus, prednisone + cyclosporine, prednisone + mizoribine.

In some embodiments, the second agent is useful for the treatment and/or prophylaxis of osteoarthritis (OA). Non-limiting examples of such agents include nonsteroidal antiinflammatory drugs (NSAIDs), topical capsaicin, intraarticular glucocorticoid injections, acetaminophen, duloxetine, tramadol, and injectable corticosteroids such as methylprednisolone acetate, triamcinolone acetate, betamethasone acetate and betamethasone sodium phosphate, triamcinolone hexacetonide, and dexamethasone.

In some embodiments, the second agent is useful for the treatment and/or prophylaxis of a gastroenterologic condition such as ulcerative colitis (UC) or Crohn’s disease (CD). Non-limiting examples of such agents include infliximab, adalimumab, golimumab, vedolizumab, tofacitinib, ustekinumab, natalizumab, mesalamine, diazo-bonded 5-ASA, sulfasalazine, balsalazide, olsalazine, corticosteroids such as budesonide, hydrocortisone, methylprednisolone, and prednisone; immunosuppressants or immunomodulators such as azathioprine and 6-mercaptopurine, cyclosporine, and methotrexate.

In some embodiments, the second agent is useful for the treatment and/or prophylaxis of a pulmonologic condition, such as idiopathic pulmonary fibrosis (IPF) or interstitial lung disease (ILD). Non-limiting examples of such agents include nitendanib, pirfenidone, corticosteroids such as prednisone, other rheumatologic drugs, including mycophenolate (e.g., CellCept®), azathioprine (e.g., Imuran®), leflunomide (e.g., ARAVA®), rituximab (e.g., RITUXAN®), cyclophosphamide (e.g., CYTOXAN®), tacrolimus (e.g., PROGRAF®), medications that reduce stomach acid, such as H-2-receptor antagonists or proton pump inhibitors such as lansoprazole (e.g., PREVACID®24HR), omeprazole (e.g., Prilosec OTC) and pantoprazole (e.g., PROTONIX®).

In some embodiments, the second agent is useful for the treatment and/or prophylaxis of a heptatologic or nephrologic condition, such as NAFLD, NASH, DKD, or CKD. Non-limiting examples of such agents include metformin, sodium-glucose cotransporter-2 inhibitor (SGLT2i), drug therapy for glycemic control, DPP-4 inhibitor, insulin, sulfonylurea, TZD (thiazolidinedione), alpha-glucosidase inhibitor, SGLT2 inhibitor (e.g., empagliflozin, canagliflozin, dapaglifloz), glucagon-like peptide-1 receptor agonist (GLP-1 RA) (e.g., lixisenatide, liraglutide, semaglutide, exenatide, albiglutide, dulaglutide), DPP-4 inhibitors (e.g., saxagliptin, alogliptin, sitagliptin, linagliptin), one or more agents used to treat high blood pressure such as angiotensin-converting enzyme (ACE) inhibitors and angiotensin 2 receptor blockers (ARBs), agents supportive of weight loss or for control of blood sugar, cholesterol-lowering drugs (e.g., statins), finerenone, and agents for treatment of diabetes mellitus, such as alpha-glucosidase inhibitors (e.g., acarbose, miglitol, voglibose).

In some embodiments, the second agent is useful for the treatment and/or prophylaxis of a dermatologic condition, such as atopic dermatitis (AD). Non-limiting examples of such agents include topical corticosteroids (TCS) (e.g., desonid, hydrocortisone, fluocinolone, triamcinolone, betamethasone diproprionate), topical calcineurin inhibitors (TCI) (e.g., tacrolimus, pimecrolimus), topical antimicrobials and antiseptics, cyclosporine, methotrexate, mycophenolate mofetil, interferon gamma, phosphodiesterase 4 (PDE4) inhibitor such as crisaborole, JAK inhibitor (e.g., ruxolitinib, upadacitinib, abrocitinib), systemic glucocorticoids (e.g., prednisone), dupilumab, and anti-IL-13 antibody (e.g., tralokinumab).

Still other aspects of the invention provide for the use of the disclosed antibodies for detecting the presence of PD-1 in biological samples. The amount of PD-1 detected may be correlated with the expression level of PD-1, which, in turn, is correlated with the activation status of immune cells (e.g., activated T cells, B cells, and monocytes) in the subject.

Specific dose of an antibody disclosed herein to be administered to a subject for treatment can vary depending on the particular antibody chosen, the dosing regimen to be followed, whether it is administered in combination with other agents, timing of administration, the tissue to which it is administered, and the physical delivery system in which it is carried. In some embodiments, an antibody is administered to a subject within a range of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 79, 80, 81, 82, 83, 84, 85, 86, 87, 88, 89, 90, 91, 92, 93, 94, 95, 96, 97, 98, 99, or 100 mg per week on average over the course of a treatment cycle.

In some embodiments, an antibody is administered to a subject in an amount greater than 1, 1.5, 2, 2.5, 3, 3.5, 4, 4.5, 5, 5.5, 6, 6.5, 7, 7.5, 8, 8.5, 9, 9.5, or 10 mg per day on average over the course of a treatment cycle. For example, the antibody is administered to a subject in an amount between about 6 and 10 mg, between about 6.5 and 9.5 mg, between about 6.5 and 8.5 mg, between about 6.5 and 8 mg, or between about 7 and 9 mg per day on average over the course of a treatment cycle.

In some embodiments, a single dose of an antibody is administered to a subject is within a range of about 0.01 mg/kg-50mg/kg, such as about, less than about, or more than about, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, or 50 mg/kg. In some embodiments, a single dose of an antibody is administered to a subject is within a range of about 0.01 mg/kg-10mg/kg, such as 0.01 mg/kg-0.1 mg/kg, 0.01 mg/kg-1 mg/kg, 0.01 mg/kg-0.5 mg/kg, 0.05 mg/kg-0.1 mg/kg, 0.05 mg/kg-0.5 mg/kg, 0.05 mg/kg-1mg/kg, 0.05 mg/kg-5mg/kg, 0.1 mg/kg-0.5 mg/kg, 0.1 mg/kg-1mg/kg, 0.1 mg/kg-5mg/kg, 0.1 mg/kg-10mg/kg, 0.5 mg/kg-1mg/kg, 0.5 mg/kg-5mg/kg, 0.5 mg/kg-10mg/kg, 1 mg/kg-5mg/kg, 1 mg/kg-10mg/kg, or 5 mg/kg-10mg/kg. A dose of the antibody may be about, at least about, or at most about 0.1, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 mg or mg/kg, or any range derivable therein. It is contemplated that a dosage of mg/kg refers to the mg amount of antibody per kg of total body weight of the subject. It is contemplated that when multiple doses are given to a patient, the doses may vary in amount or they may be the same.

In some embodiments, an antibody is administered to a subject within a range of about 0.01 mg/kg-50mg/kg per day, such as about, less than about, or more than about, 0.01 mg/kg, 0.02 mg/kg, 0.03 mg/kg, 0.04 mg/kg, 0.05 mg/kg, 0.06 mg/kg, 0.07 mg/kg, 0.08 mg/kg, 0.09 mg/kg, 0.1 mg/kg, 0.2 mg/kg, 0.3 mg/kg, 0.4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, 20 mg/kg, 25 mg/kg, 30 mg/kg, 35 mg/kg, 40 mg/kg, 45 mg/kg, or 50 mg/kg per day. In some embodiments, an antibody is administered to a subject within a range of about 0.1 mg/kg-400mg/kg per week, such as about, less than about, or more than about 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 100 mg/kg, 150 mg/kg, 200 mg/kg, 250 mg/kg, 300 mg/kg, 350 mg/kg, or 400 mg/kg per week. In some embodiments, an antibody is administered to a subject within a range of about 0.4 mg/kg-1500 mg/kg per month, such as about, less than about, or more than about 0.4 mg/kg, 0.5 mg/kg, 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 100 mg/kg, 150 mg/kg, 200 mg/kg, 250 mg/kg, 300 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, or 1000 mg/kg per month. In some embodiments, an antibody is administered to a subject within a range of about 0.1 mg/m²-200 mg/m² per week, such as about, less than about, or more than about 1 mg/m², 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 100 mg/m², 125 mg/m², 150 mg/m², 175 mg/m², or 200 mg/m² per week. The target dose may be administered in a single dose. Alternatively, the target dose may be administered in about or more than about 1, 2, 3, 4, 5, 6, 7, 8, 9, 10, 11, 12, 13, 14, 15, 16, 17, 18, 19, 20, 25, 30, or more doses. For example, a dose of about 1 mg/kg per week may be delivered weekly at a dose of about 1 mg/kg every week, about 2 mg/kg administered every two weeks, or about 4 mg/kg administered every four weeks over the course of the week. The administration schedule may be repeated according to any regimen as described herein, including any administration schedule described herein. In some embodiments, an antibody is administered to a subject in the range of about 0.1 mg/m²-500mg/m², such as about, less than about, or more than about 1 mg/m², 5 mg/m², 10 mg/m²,15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 100 mg/m², 130 mg/m², 135 mg/m², 155 mg/m², 175 mg/m², 200 mg/m², 225 mg/m², 250 mg/m², 300 mg/m², 350 mg/m², 400 mg/m², 420 mg/m², 450 mg/m², or 500 mg/m².

### Pharmaceutical Composition

In another aspect, provided herein are pharmaceutical compositions comprising the anti-PD-1 antibody or a functional fragment thereof disclosed herein, and a pharmaceutically acceptable carrier or excipient. The pharmaceutically acceptable carrier or excipient can include, but not limited to, inert solid diluents and fillers, diluents, sterile aqueous solution and various organic solvents, permeation enhancers, solubilizers and adjuvants. These compositions can be formulated according to known methods for preparing pharmaceutically useful compositions. Formulations are described in a number of sources which are well known and readily available to those skilled in the art. For example, Remington’s Pharmaceutical Science (Martin E.W., Easton Pennsylvania, Mack Publishing Company, 19^{th} ed., 1995) describes formulations which can be used in connection with the subject invention.

The pharmaceutical composition disclosed herein can, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. Suitable examples of sustained release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. Some sustained release formulations enable release of molecules over a few weeks to a few months, or even up to a few years. In some embodiments, the subject pharmaceutical composition release the subject antibody as described herein for at least a few weeks, such as for at least 1 week, 2 weeks, 3 weeks or 4 weeks. In further embodiments, the subject pharmaceutical composition release the subject antibody as described herein over a few months, such as for at least 1 month, 2 months, 3 months, 4 months, 5 months, or 6 months.

The pharmaceutical composition disclosed herein can be in unit dosage forms suitable for single administration of precise dosages. The pharmaceutical composition can further comprise an antibody or a functional fragment thereof as an active ingredient and may include a conventional pharmaceutical carrier or excipient. Further, it may include other medicinal or pharmaceutical agents, carriers, adjuvants, etc.

Exemplary parenteral administration forms include solutions or suspensions of active polypeptide and/or PEG-modified polypeptide in sterile aqueous solutions, for example, aqueous propylene glycol or dextrose solutions. Such dosage forms can be suitably buffered with salts such as histidine and/or phosphate, if desired.

Formulations suitable for administration include, for example, aqueous sterile injection solutions, which may contain antioxidants, buffers, bacteriostats, and solutes which render the formulation isotonic with the blood of the intended recipient; and aqueous and nonaqueous sterile suspensions which may include suspending agents and thickening agents.

The formulations may be presented in unit-dose or multi-dose containers, for example sealed ampoules and vials, and may be stored in a freeze dried (lyophilized) condition requiring only the condition of the sterile liquid carrier, for example, water for injections, prior to use. Extemporaneous injection solutions and suspensions may be prepared from sterile powder, granules, tablets, etc.

In some embodiments, the disclosure provides a pharmaceutical composition for injection containing a subject antibody or a functional fragment thereof and a pharmaceutical excipient suitable for injection. Example components and amounts of agents in such compositions are as described herein.

The forms in which the compositions of the present disclosure may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline can be used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions can be prepared by incorporating an antibody of the present disclosure or functional fragment thereof in the desired amount in the appropriate solvent with various other ingredients as enumerated above, followed by filtered sterilization. Generally, dispersions are prepared by incorporating the various sterilized active ingredients into a sterile vehicle which contains the basic dispersion medium and other ingredients. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

In some embodiments, the disclosure provides a pharmaceutical composition for oral administration containing an antibody of the present disclosure or a functional fragment thereof, and a pharmaceutical excipient suitable for oral administration.

In some embodiments, a solid pharmaceutical composition for oral administration is provided herein containing: (i) an effective amount of an antibody of the present disclosure or a functional fragment thereof; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition is a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, and typically include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation.

This disclosure further encompasses anhydrous pharmaceutical compositions and dosage forms comprising an active ingredient, since water can facilitate the degradation of some polypeptides. For example, water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An antibody of the present disclosure can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants can be used in the compositions to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, exemplary lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In some cases the composition includes a solubilizer to ensure good solubilization and/or dissolution of the compound and to minimize precipitation of the compound. This can be especially advantageous for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Exemplary solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

The composition can further include one or more pharmaceutically acceptable additives and excipients. Such additives and excipients include, without limitation, detackifiers, antifoaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

In another aspect of the disclosure, provided are kits comprising the unit doses containing the antibody compositions of the disclosure and instructions for use. The kit can further comprise one or more unit doses containing one or more additional reagents, such as an immunosuppressive reagent as described above, or one or more additional antibodies as described herein (e.g., a human antibody having a complementary activity which binds to an epitope in the antigen distinct from a first human antibody). Kits typically include a label indicating the intended use of the contents of the kit. The term label includes any writing, or recorded material supplied on or with the kit, or which otherwise accompanies the kit.

A kit of the present disclosure may also include diagnostic agents and/or other therapeutic agents. In some cases a kit includes an antibody of the present disclosure and a diagnostic agent that may be used in a diagnostic method for diagnosing the state or existence of a disease, condition or disorder in a subject as described herein.

### Medical Use

In another aspect, provided herein is an antibody or an antigen-binding fragment or an immunoconjugate of the present disclosure, a pharmaceutical composition comprising an antibody or an antigen-binding fragment or an immunoconjugate of the present disclosure for use in therapy. Suitably, provided herein is an antibody or an antigen-binding fragment or an immunoconjugate of the present disclosure, or a pharmaceutical composition comprising an antibody or an antigen-binding fragment or immunoconjugate of the present disclosure for use in a method of treatment as disclosed herein.

In another aspect, provided herein is the use of an antibody or an antigen-binding fragment or an immunoconjugate of the present disclosure, or pharmaceutical composition comprising an antibody or an antigen-binding fragment or immunoconjugate of the present disclosure in the manufacture of a medicament for use in therapy, such as for use in a method of treatment as disclosed herein.

### Sequence Listing

SEQ ID NO: 1, CDRH1, molecular type: protein, organism: synthetic construct

TYPIE

SEQ ID NO: 2, CDRH2, molecular type: protein, organism: synthetic construct

NFHPYNDDTKYNEKFQG

SEQ ID NO: 3, CDRH3, molecular type: protein, organism: synthetic construct

ENYGSHGGFVY

SEQ ID NO: 4, CDRL1, molecular type: protein, organism: synthetic construct

RASSSVISSYLH

SEQ ID NO: 5, CDRL2, molecular type: protein, organism: synthetic construct

STSNLAS

SEQ ID NO: 6, CDRL3, molecular type: protein, organism: synthetic construct

QQYNSYPLT

SEQ ID NO: 17, Fc of human IgG1 with P238D mutation, molecular type: protein, organism: synthetic construct

THTCPPCPAPELLGGDSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNV
FSCSVMHEALHNHYTQKSLSLSPGK

SEQ ID NO: 18, heavy chain, molecular type: protein, organism: synthetic construct

QVQLVQSGAEVKKPGASVKVSCKAFGYTFTTYPIEWMRQAPGKGLEWIGN
FHPYNDDTKYNEKFQGRVTLTVDKSSTTVYMELSSLRSEDTAVYYCAREN
YGSHGGFVYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGDSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

SEQ ID NO: 19, light chain, molecular type: protein, organism: synthetic construct

ENQLTQSPSSLSASVGDRVTITCRASSSVISSYLHWYQQKPGKAPKLLIY
STSNLASGVPSRFSGSGSGTDYTLTISSLQPEDFATYYCQQYNSYPLTFG
GGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Effect of Fc Receptor Interaction on Agonist Function of Exemplary Anti-PD-1 Antibody

The signaling effects of an exemplary anti-human PD-1 agonist antibody (Clone 19 mouse IgG1 described in U.S. Patent No. 9,181,342, issued Nov. 10, 2015) were explored in a reporter assay in which PD-1 expressing Jurkat T cells, which produce luciferase under the control of an NFAT response-element, were cultured with BW5147 cells expressing an anti-CD3 ‘T cell stimulator’ (TCS) construct as previously described (Leitner et al. 2010). To explore the role of Fc receptor interaction in antibody agonism the assay was performed either with BW5147 cells expressing only the TCS construct or with BW5147 cells also transfected to express mouse FcyR2B.

5×10⁴ Jurkat reporter cells (Promega cat# J1250b) were added per well, in a 96 well U-bottom plate, and cocultured with 5×10⁴ BW5147 cells, plus either PD-1 antibody or isotype control, in a total volume of 80µL assay buffer (RPMI 1640 + 1% FCS). A 9 point dilution series of antibody was performed with 1 in 3 dilutions down from 200 nM. After 6 hours incubation in a humidified CO₂ incubator at 37° C., plates were removed from the incubator and equilibrated to room temperature for 10 minutes. The amount of luciferase produced was quantified (as a measure of T cell activation) using Bio-Glo™ Luciferase Assay System (Promega); 80 µl Bio-Glo™ Luciferase Assay Reagent was added to each well and plates were incubated for 10 min at room temperature. Luminescence was quantified using a CLARIOstar Plus (BMG Labtech).

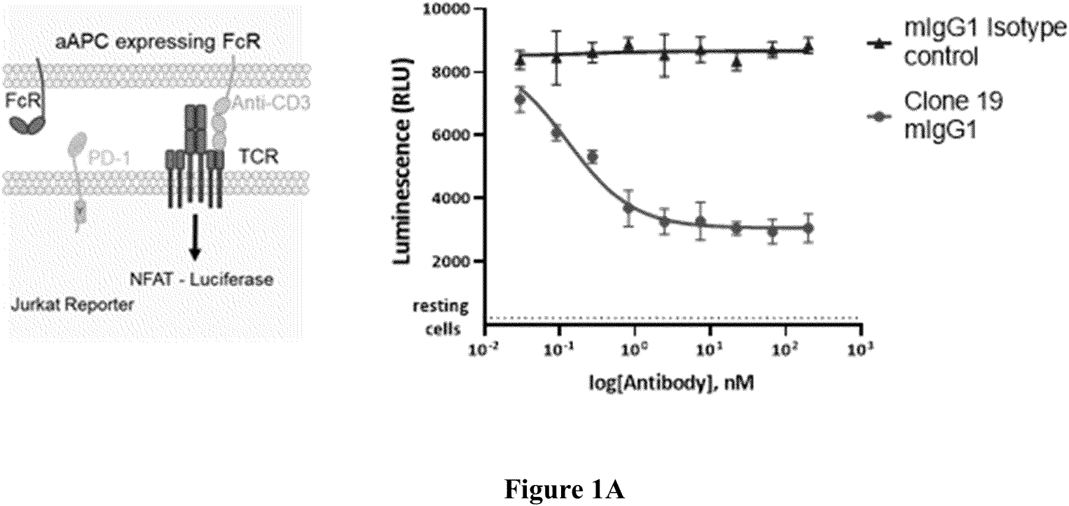

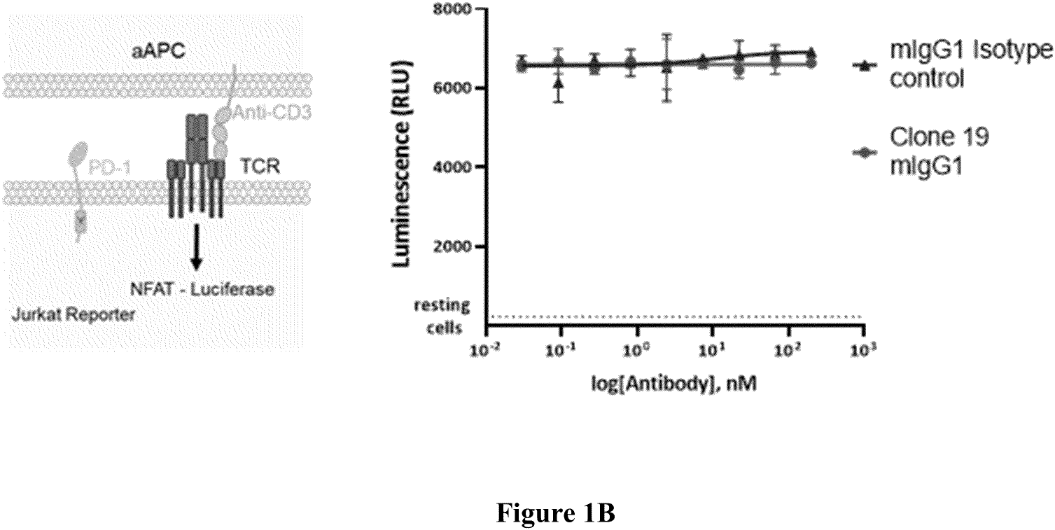

When stimulator cells that expressed mouse FcyR2B were used, the PD-1 antibody Clone 19 caused a significant reduction in T cell activation with an IC50 of 0.13 nM (FIG. 1A). When stimulator cells that expressed no Fc receptor were used, Clone 19 had no impact on T cell activation (FIG. 1B).

### Example 2: Humanization of Exemplary Anti-PD-1 Antibody

The VH and VL sequences of Clone 19 were aligned to a database of human germline sequences and homologous sequences were selected as frameworks for humanization. IGHV1-24*01 or IGHV7-4-1*02 were used as frameworks for the VH domain and IGKV1-39*01 or IGKV3-11*01 were used as frameworks for the VL domain.

The VH and VL sequences were run through a CDR grafting algorithm to transfer the CDRs from the murine antibody Clone 19 onto the selected human germline sequences. To enable structure guided humanization, models were built for the Clone 19 murine VH and VL and a structure guided approach was used to determine which of the framework amino acids to retain in the humanized antibody frame for the sake of retaining binding integrity. Table 1 summarizes the VH and VL sequences that were generated.

Antibody variants were produced consisting of all the potential combinations of humanized VH and VL domains. Variants were produced on a human IgG1 kappa isotype. The binding affinity and kinetics of the humanized antibody variants to human or cynomolgus PD-1 were determined by surface plasmon resonance (SPR) using the Biacore 8 K (Cytiva). Human antibody capture kit (Cytiva) was used to coat a Series S CM5 Sensor Chip (Cytiva) with polyclonal anti-human IgG. Anti-PD-1 antibody was then captured onto the biosensor surface and an isotype control antibody captured in the reference channel. Various concentrations of monomeric soluble human PD-1 extracellular domain or soluble cynomolgus macaque PD-1 extracellular domain were then injected over the immobilized antibodies in the buffer 10 mM Hepes, 150 mM NaCl, 0.005% v/v Surfactant P20, pH 7.4 (HBS-P) at 37° C., in a single cycle kinetics analysis. Association and dissociation rates were fitted using BiaEvaluation Software (Cytiva) after reference and blank subtractions, and dissociation constants were calculated. Table 2 shows the binding K_{D} for each of the humanized variants to human and cynomolgus PD-1.

TABLE 1

Sequences of Exemplary PD-1 Agonist Antibody Variable Regions

| | Name | Human framework | Sequence | SEQ ID NO | Mole cular type | Orga nism |
|---|---|---|---|---|---|---|
| Parental | VH | - | QVQLQQSGAELVKPGASVKMSCKAFG YTFTTYPIEWMKQNHGKSLEWIGNFHP YNDDTKYNEKFKGKAKLTVEKSSTTV YLELSRLTSDDSAVYYCARENYGSHG GFVYWGQGTLVTVSG | 7 | Prote in | synth etic const ruct |
| Humani zed | hu-VH1 | IGHV1-24*01 | QVQLVQSGAEVKKPGASVKVSCKAFG YTFTTYPIEWMRQAPGKGLEWIGNFHP YNDDTKYNEKFQGRVTLTVDKSSTTV YMELSSLRSEDTAVYYCARENYGSHG GFVYWGQGTLVTVSS | 8 | Prote in | synth etic const ruct |
| Humani zed | hu-VH2 | IGHV1-24*01 | QVQLVQSGAEVKKPGASVKVSCKAFG YTFTTYPIEWVRQAPGKGLEWMGNFH PYNDDTKYNEKFQGRVTMTVDKSTTT VYMELSSLRSEDTAVYYCARENYGSH GGFVYWGQGTLVTVSS | 9 | Prote in | synth etic const ruct |
| Humani zed | hu-VH3 | IGHV7-4-1*02 | QVQLVQSGSELKKPGASVKVSCKAFG YTFTTYPIEWMRQAPGQGLEWIGNFHP YNDDTKYNEGFTGRFVLSVDKSSTTV YLQISSLKAEDTAVYYCARENYGSHG GFVYWGQGTLVTVSS | 10 | Prote in | synth etic const ruct |
| Humani zed | hu-VH4 | IGHV7-4-1*02 | QVQLVQSGSELKKPGASVKVSCKAFG YTFTTYPIEWVRQAPGQGLEWMGNFH PYNDDTKYNEGFTGRFVFSVDKSVTT VYLQISSLKAEDTAVYYCARENYGSH GGFVYWGQGTLVTVSS | 11 | Prote in | synth etic const ruct |
| Parental | VL | - | ENVLTQSPAIMSASPGEKVTMTCRASS SVISSYLHWYQQKSGASPKLWIYSTSN LASGVPDRFSGSGSGTSYSLTISSVEAE DAATYYCQQYNSYPLTFGAGTKLEIK | 12 | Prote in | synth etic const ruct |
| Humani zed | hu-VL1 | IGKV1-39*01 | ENQLTQSPSSLSASVGDRVTITCRASSS VISSYLHWYQQKPGKAPKLLIYSTSNL ASGVPSRFSGSGSGTDYTLTISSLQPED FATYYCQQYNSYPLTFGGGTKLEIK | 13 | Prote in | synth etic const ruct |
| Humani zed | hu-VL2 | IGKV1-39*01 | ENQLTQSPSSLSASVGDRVTITCRASSS VISSYLHWYQQKPGKAPKLLIYSTSNL ASGVPSRFSGSGSGTDYTLTISSLQPED FATYYCQQYRGYPLTFGGGTKLEIK | 14 | Prote in | synth etic const ruct |
| Humani zed | hu-VL3 | IGKV3-11*01 | ENVLTQSPATLSLSPGERATLSCRASSS VISSYLHWYQQKPGQAPRLLIYSTSNL ASGIPARFSGSGSGTDYTLTISSLEPEDF AVYYCQQYNSYPLTFGGGTKLEIK | 15 | Prote in | synth etic const ruct |
| Humani zed | hu-VL4 | IGKV3-11*01 | ENVLTQSPATLSLSPGERATLSCRASSS VISSYLHWYQQKPGQAPRLLIYSTSNR ATGIPARFSGSGSGTDYTLTISSLEPEDF AVYYCQQYNSYPLTFGGGTKLEIK | 16 | Prote in | synth etic const ruct |

TABLE 2

Binding Affinity of Exemplary PD-1 Agonist Antibody

| Antibody ID | VH name | VL name | Human PD-1 binding K_{D} (nM) | Cyno PD-1 binding K_{D} (nM) |
|---|---|---|---|---|
| Parental | VH | VL | 53.7 | 318 |
| humCL19v1 | hu-VH1 | hu-VL1 | 31.5 | 278 |
| humCL19v2 | hu-VH1 | hu-VL2 | 1010 | 3410 |
| humCL19v3 | hu-VH1 | hu-VL3 | 37.3 | 236 |
| humCL19v4 | hu-VH1 | hu-VL4 | 32 | 199 |
| humCL19v5 | hu-VH2 | hu-VL1 | 57.6 | 440 |
| humCL19v6 | hu-VH2 | hu-VL2 | 2000 | 15200 |
| humCL19v7 | hu-VH2 | hu-VL3 | 64.5 | 565 |
| humCL19v8 | hu-VH2 | hu-VL4 | 58.9 | 369 |
| humCL19v9 | hu-VH3 | hu-VL1 | 44.8 | 251 |
| humCL19v10 | hu-VH3 | hu-VL2 | 670 | 8460 |
| humCL19v11 | hu-VH3 | hu-VL3 | 52.5 | 249 |
| humCL19v12 | hu-VH3 | hu-VL4 | 47.5 | 253 |
| humCL19v13 | hu-VH4 | hu-VL1 | 160 | 1000 |
| humCL19v14 | hu-VH4 | hu-VL2 | 80200 | 79200 |
| humCL19v15 | hu-VH4 | hu-VL3 | 49000 | 101000 |
| humCL19v16 | hu-VH4 | hu-VL4 | 161 | 896 |

### Example 3: Impact of Fc Mutation on Selectivity of Binding To FcyR2B

The humanized variant humCL19v1 was recombinantly produced on a hIgG1, a hIgG4 or a range of different Fc mutated hIgG1 constant regions and their binding to human FcyR2B or the two highly homologous FcyR2A allotypes was assessed by surface plasmon resonance (at 37° C. in buffer HBS-EP+ at pH7.4).

The interactions were assessed by surface plasmon resonance using a Biacore 8 K with the recombinantly expressed FcRs (extracellular domains only) as analyte. Briefly, recombinant human PD-1 extracellular domain was covalently immobilized to both flow cells of all channels of a CM5 Series S sensor chip using the GE Healthcare Amine coupling kit. The humCL19v1 Fc variant to be assessed was then captured (approx. 500-1000 response units) in flow cell 2 of each channel. Steady state affinity analysis was then performed by injecting varying concentrations of FcR and measuring equilibrium binding. Double referencing was used (subtracting the signal in the reference Fc1 and also subtracting the signal from a blank zero concentration injection). K_{D} values were calculated from the Langmuir curves (plotting equilibrium binding against analyte concentration to determine the concentration required for half maximal binding). The binding K_{D} values for each of the variants to the different Fc receptors is shown in Table 3. Of the Fc variants tested, only the P238D mutation enhanced the selectivity of binding to FcyR2B vs both the FcyR2A allotypes. This mutation led to a modest increase in binding to FcyR2B and a significant decrease in binding to both the FcyR2A allotypes.

TABLE 3

Binding Affinity of Exemplary Antibodies

| | Binding K_{D} (nM) | | | Ratio of binding affinity FcyR2B: FcyR2A (131R) |
|---|---|---|---|---|
| | FcγR2B | FcγR2A 131R | FcγR2A 131H | |
| hIgG1 | 4.10 | 1.36 | 1.95 | 0.33 |
| hIgG4 | 3.31 | 2.55 | 7.16 | 0.77 |
| hIgG1 P238D | 2.79 | 17.50 | 90.30 | 6.27 |
| hIgG1 L235N | 59.00 | 12.30 | 13.50 | 0.21 |
| hIgG1 L235H | 13.60 | 3.67 | 3.96 | 0.27 |
| hIgG1 V266M | 1.54 | 0.58 | 6.28 | 0.37 |
| hIgG1 S239V | 3.60 | 1.62 | 3.13 | 0.45 |
| hIgG1 S239I | 3.99 | 1.65 | 3.38 | 0.41 |
| hIgG1 Y300N | 6.32 | 2.22 | 2.83 | 0.35 |
| hIgG1 P271Y | 20.60 | 9.70 | 8.85 | 0.47 |

### Example 4: P238D Mutated PD-1 Antibody Is As Effective As IgG1 Antibodies at Suppressing T Cell Activation in An NFA Reporter Assay

In order to assess whether the P238D mutation impacts the agonist function of humCL19v1 a Jurkat reporter assay was used. Comparison was made to both the unmutated IgG1 version of humCL19v1 and to IgG1 isotype agonist antibodies that have been previously described including PD1AB6 (WO 2017/058859 A1), PD1B1094 (WO 2018/226580 A2), Antibody 1 (WO 2019/168745 A1) and ANB030 (WO 2020/247628 A2). These antibodies were produced recombinantly from sequences provided in the respective patent applications. Jurkat T cells, which produce luciferase under the control of an NFAT response-element, were cultured with BW5147 cells expressing an anti-CD3 “T cell stimulator” (TCS) construct as previously described (Leitner et al. 2010) and expressing human FcyR2B.

5×10^4 Jurkat reporter cells (Promega cat# J1250b) were added per well, in a 96 well U-bottom plate, and cocultured with 5×104 BW5147 cells, plus either PD-1 antibody or isotype control, in a total volume of 80 µL assay buffer (RPMI 1640 + 1% FCS). A single high dose of each PD-1 antibody (10 µg/ml) was tested.

After 6 hours incubation in a humidified CO2 incubator at 37° C., plates were removed from the incubator and equilibrated to room temperature for 10 minutes. The amount of luciferase produced was quantified (as a measure of T cell activation) using Bio-Glo™ Luciferase Assay System (Promega); 80 µl Bio-Glo™ Luciferase Assay Reagent was added to each well and plates were incubated for 10 min at room temperature. Luminescence was quantified using a CLARIOstar Plus (BMG Labtech).

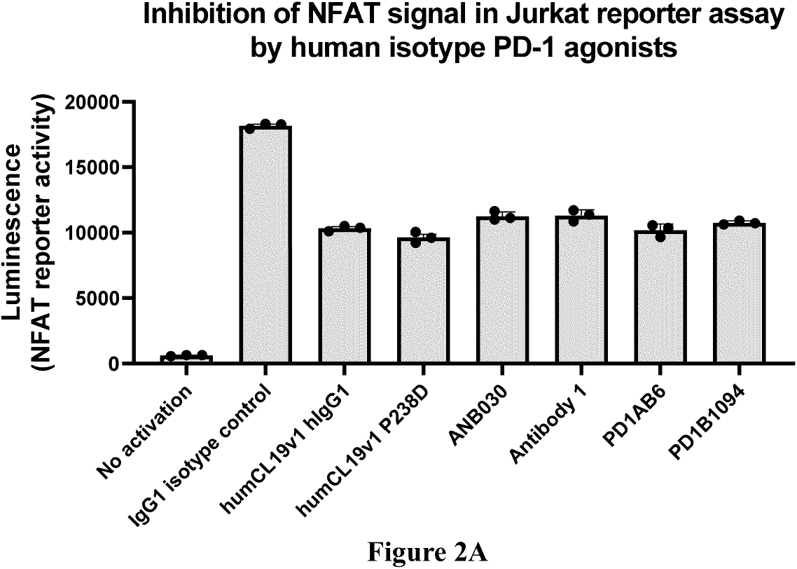

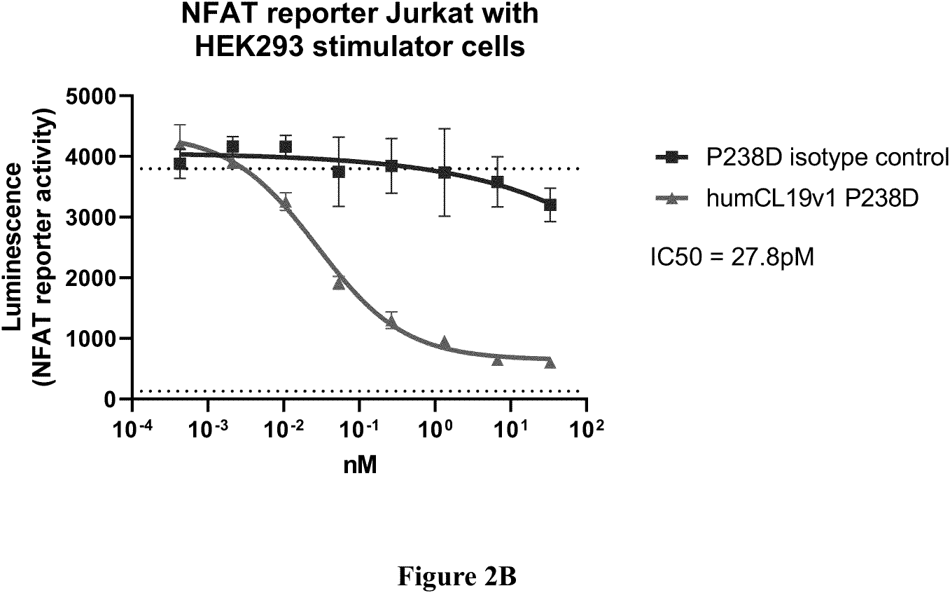

All PD-1 antibodies tested significantly reduced T cell activation compared to isotype control. There was no significant difference between wildtype IgG1 or P238D mutated versions of humCL19v1 (FIG. 2A).

In another set of experiments, an optimised T cell reporter assay was used to assess the potency of P238D mutated version of humaCL19v1. The T cell reporter assay was similar to the assay described above in this example, but the murine BW5147 stimulator cells were replaced with human HEK293T stimulator cells. Jurkat T cells, which produce luciferase under the control of an NFAT response-element, were cultured with HEK293T cells expressing an anti-CD3 “T cell stimulator” (TCS) construct as previously described (Leitner et al. 2010) and expressing human FcyR2B.

4×10^4 HEK293T stimulator cells were plated per well in flat bottom 96 well plates. After 16 hours the media was removed and 5×10^4 Jurkat reporter cells (Promega cat# J1250b) were added per well, plus either PD-1 antibody or isotype control, in a total volume of 80 µL assay buffer (RPMI 1640 + 1% FCS). A 5-fold serial dilution of antibody was tested starting at 5 µg/ml.

After 6 hours incubation in a humidified CO₂ incubator at 37° C., plates were removed from the incubator and equilibrated to room temperature for 10 minutes. The amount of luciferase produced was quantified (as a measure of T cell activation) using Bio-Glo™ Luciferase Assay System (Promega); 80 µl Bio-Glo™ Luciferase Assay Reagent was added to each well and plates were incubated for 10 min at room temperature. Luminescence was quantified using a CLARIOstar Plus (BMG Labtech). As shown in FIG. 2B, humCL19v1 P238D inhibited T cell activation by up to 84%, as assessed by NFAT induced luminescent signal, with an IC50 of 0.0278 nM.

### Example 5: P238D Mutated PD-1 Antibody Is As Effective As IgG1 Antibodies at Suppressing Primary T Cell Activation in T Cell Activation Assays

In one set of experiments, a tetanus toxoid activation assay was used to assess inhibitory effects of exemplary antibodies on T cell activation. Total human peripheral blood mononuclear cells (PBMCs) from healthy donors (400,000 cells per well of a 96 U-bottom plate) were stimulated with Tetanus Toxoid (0.5 µg/mL) in the presence of PD-L½-blocking antibodies (5 µg/mL each) and 1 µg/ml of PD-1 agonist antibody or isotype control. IFNγ release was assessed by ELISA of supernatant after 96 hours incubation at 37° C., 5% CO2. 6 donors were assessed and data was collated by normalizing in each donor to the IFNg level in cells activated with Tetanus Toxoid in the absence of test antibody.

Antibodies tested included the P238D mutated humCL19v1, the unmutated IgG1 version of humCL19v1 and IgG1 isotype agonist antibodies that have been previously described including PD1AB6 (WO 2017/058859 A1) and Antibody 1 (WO 2019/168745 A1). These antibodies were produced recombinantly from sequences provided in the respective patents.

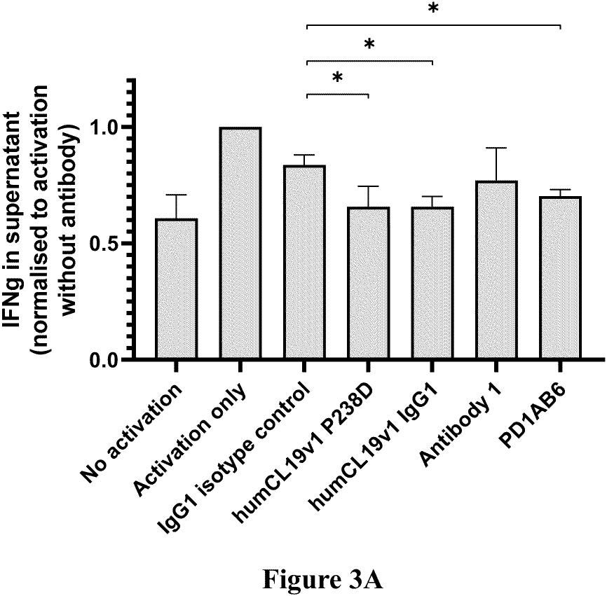

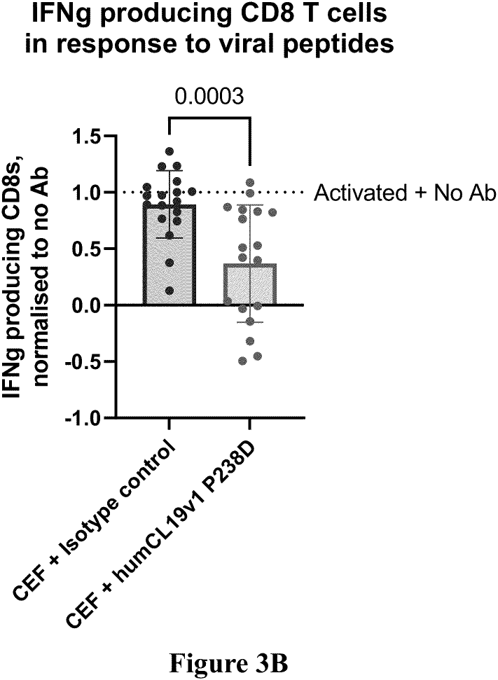

Averaged across the donors tested, tetanus toxoid (TT) induced an approximately 2 fold increase in IFNg production compared to PBMCs culture without TT. The IgG1 isotype control slightly reduced IFNg production. humCL19v1 P238D, humCL19v1 IgG1 and PD1AB6 all significantly reduced IFNg production compared to the isotype control. There was no significant difference between wildtype IgG1 or P238D mutated versions of humCL19v1 (FIG. 3A).

In another set of experiments, a viral peptide activation assay was used to assess inhibitory effects of exemplary antibodies on immune cell activation. Total human peripheral blood mononuclear cells (PBMCs) from healthy donors (500,000 cells per well of a 96 U-bottom plate) were stimulated with 2 µg/mL CEF HLA Class I peptides (A pooled mixture of peptides from cytomegalovirus, Epstein-Barr virus and influenza, Mabtech cat# 3618-1) in the presence of 5 µg/mL Brefeldin A (Biolegend cat# 420601) and 1 µg/ml PD-1 antibody or P238D mutated hIgG1 isotype control, or no antibody. The percentage of IFNγ producing cells within the CD8 T cell population was assessed using intracellular flow cytometry after 16 hours incubation at 37° C., 5% CO2. 18 donors were assessed. Data was collated by normalizing in each donor using the formula below:

(Cytokine production in presence of antibody — unstimulated background) / (Cytokine production in stimulated cells with no antibody — unstimulated background).

Averaged across the donors tested, CEF peptides induced a 4 fold increase in CD8 IFNg producing T cell compared to PBMCs culture without CEF peptides. humCL19v1 P238D significantly reduced IFNg, on average by 63% compared to the no antibody control. (FIG. 3B).

### Example 6: P238D Mutated PD-1 Antibody Is As Effective As IgG1 Antibodies at Suppressing Primary T Cell Activation in An Anti-CD3/28 Activation Assay

Total human peripheral blood mononuclear cells (PBMCs) from healthy donors (100,000 cells per well of a 96 U-bottom plate) were stimulated with soluble anti-CD3 and anti-CD28 antibodies (0.5 ng/mL final concentration of each) in the presence of 1 µg/ml PD-1 agonist antibody or isotype control. CD25 expression on CD4 T cells was assessed by flow cytometry as a marker of T cell activation after 72 hours incubation at 37° C., 5% CO₂. Data was collated from multiple donors by normalizing in each donor to the CD25 geomean in cells activated with anti-CD3 and anti-CD28 in the absence of test antibody.

Antibodies tested included the P238D mutated humCL19v1 and IgG1 agonist antibodies that have been previously described including PD1AB6 (WO 2017/058859 A1) and Antibody 1 (WO 2019/168745 A1). These antibodies were produced recombinantly from sequences provided in the respective patents.

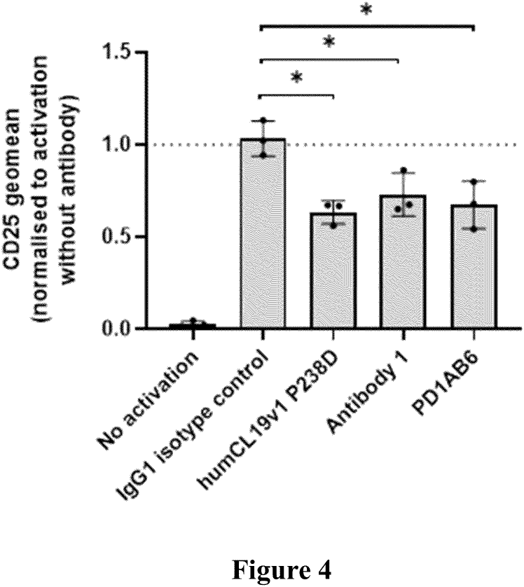

Anti-CD3 and anti-CD28 led to a significant increase in CD25 expression that was significantly inhibited by all PD-1 antibodies tested (FIG. 4).

### Example 7: IgG1 Isotype Anti-PD-1 Antibodies Lead To ADCC Killing of Regulatory T Cells In Vitro, But P238D Mutated PD-1 Antibody Does Not

An in vitro NK cell degranulation assay was used to study the potential of anti-PD-1 antibodies to deplete regulatory T cells by ADCC. Tregs were purified by magnetic isolation from PBMCs of healthy donors using the human CD4+ CD25+ CD 127dim/- Regulatory T Cell Isolation Kit II from Miltenyi (cat# 130-094-775). NK cells were similarly purified using the human NK isolation kit from Miltenyi (cat# 130-092-657).

20,000 isolated NK cells were plated per well in a 96-well U-bottom plate with 1 ug/ml of different anti-PD-1 antibodies or IgG1 isotype control. Antibodies tested included the P238D mutated humCL19v1, the unmutated IgG1 version of humCL19v1, and IgG1 isotype agonist antibodies that have been previously described including PD1B1094 (WO 2018/226580 A2) and Antibody 1 (WO 2019/168745 A1). These antibodies were produced recombinantly from sequences provided in the respective patent applications.

100,000 Treg cells were added per well to give an effector: target ratio of 1:5. Finally, anti-CD107a antibody (Biolegend #328638) was added to each well for a final dilution of 1 in 100, along with Monensin (Biolegend #420701) and Brefeldin (Biolegend #420601) to give a final 1x concentration of each. The final well volume was 200 ul. The assay was incubated for 6 hours at 37° C. in 5% CO2. The assay was then stained with an antibody panel including CD3, a dead cell marker, and CD56, fixed with 1% formaldehyde and assessed by flow cytometry. Data generated was analyzed with FlowJo V10. Degranulated NK cells were identified as CD107+CD56+ cells. Dead Treg cells were identified as CD3+ cells positive for the dead cell marker.

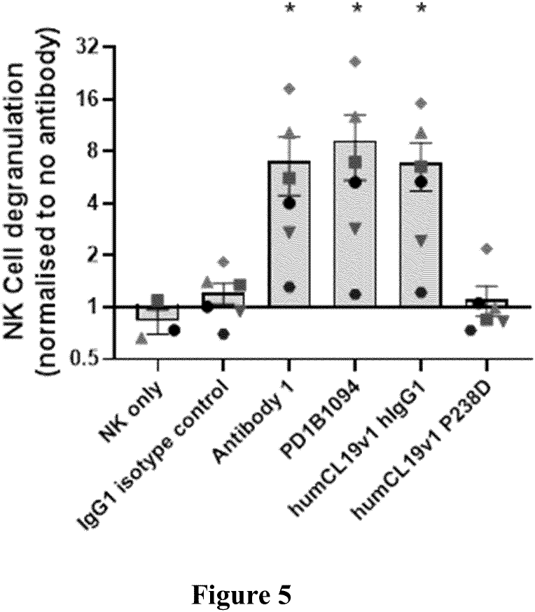

IgG1 isotype anti-PD-1 antibodies led to significant activation of NK cell degranulation (FIG. 5). P238D mutated humCL19v1 caused no NK cell degranulation or Treg death.

### Example 8: HumCL19v1 P238D Is Able To Further Inhibit T Cell Activation In the Presence of High Levels of PD-L1, While Other PD-1 Agonist Antibodies Are Not

A PD-1 Reporter cell line was used to assess the impact of the P238D mutated humCL19v1 compared to previously described PD-1 agonist antibodies in a setting where PD-L1 is highly expressed.

PD-L1 expressing CHOK1 cells that express a T-cell stimulator construct (Promega cat# J1250a) were plated at 40,000 cells per well in 96 well flat bottom plate and incubated overnight to adhere to the plate. The next day supernatant was removed and 50,000 PD-1 expressing Jurkat reporter cells, expressing luciferase under the control of an NFAT response element, were added alongside PD-1 antibody or isotype control. Dose titrations of PD-1 antibodies were assessed using 4x dilutions down from 10 ug/ml in a total volume per well of 80 µl. After a 6 hour incubation at 37C, 80 µl Bio-Glo was added per well and incubated for 15 mins, then read on a Clariostar plate reader using the Firefly Luciferase setting to quantify luciferase production.

Antibodies tested included the P238D mutated humCL19v1 and IgG1 isotype agonist antibodies that have been previously described including PD1AB6 (WO 2017/058859 A1), PD1B1094 (WO 2018/226580 A2), Antibody 1 (WO 2019/168745 A1) and ANB030 (WO 2020/247628 A2). These antibodies were produced recombinantly from sequences provided in the respective patents. As a control, a biosimilar of the PD-1 blocking antibody Nivolumab was also assessed.

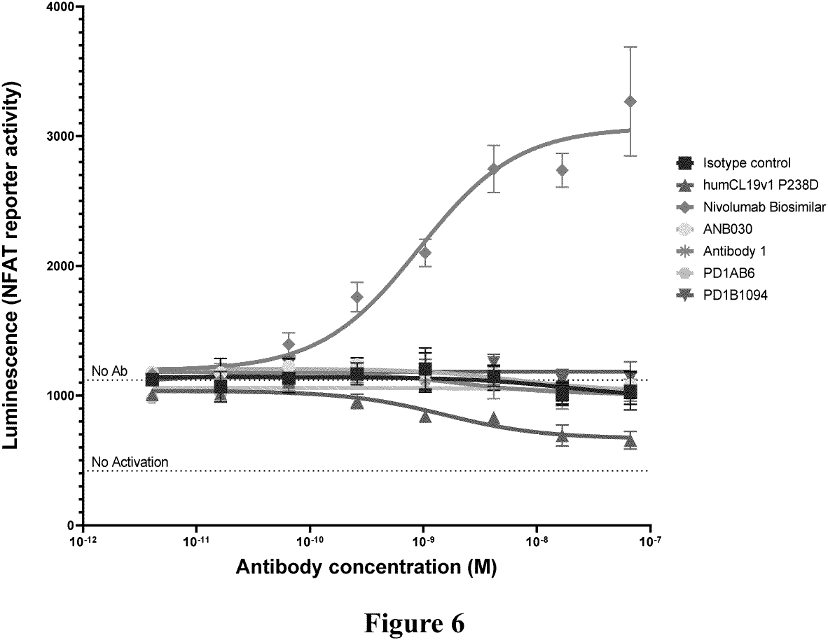

As expected Nivolumab led to a significant dose responsive increase in T cell activation, by blocking the interaction of PD-L1 with PD-1. Unexpectedly, only the P238D mutated humCL19v1 antibody demonstrated an ability to further suppress T cell activation (beyond the inhibition already provided by the PD-L1 interaction with PD-1). None of the other PD-1 antibodies tested had any impact on T cell activation (FIG. 6).

### Example 9: humCL19v1 P238D Is Able To Inhibit T Cell Activation In RA PBMC, Fibroblast Co-Cultures

In order to study the impact of PD-1 agonists on T cells from rheumatoid arthritis (RA) patients, PBMCs from 4 donors with RA were activated in a co-culture setting with RA fibroblast like synoviocytes (FLS, from Tebu-bio #408RAK-05a). Stromal cells such as fibroblasts express PD-L1 and PDL-2, and so this assay represents a physiological situation in which PD-1′s ligands are present (Dezutter-Dambuyant et al. 2016).

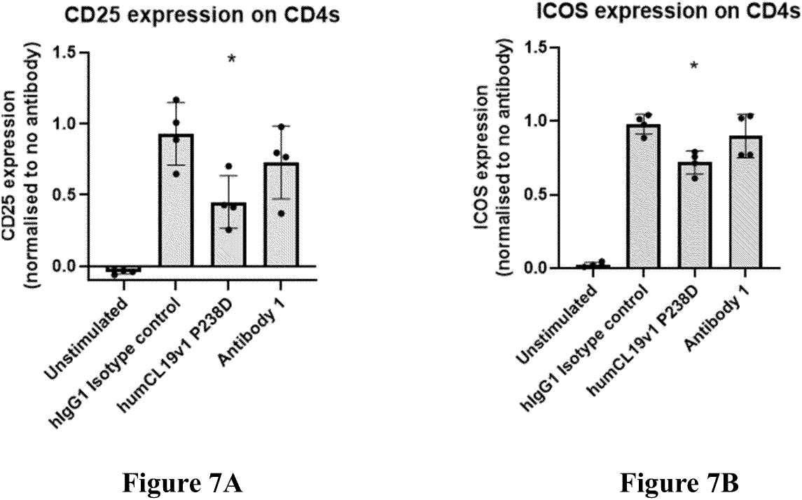

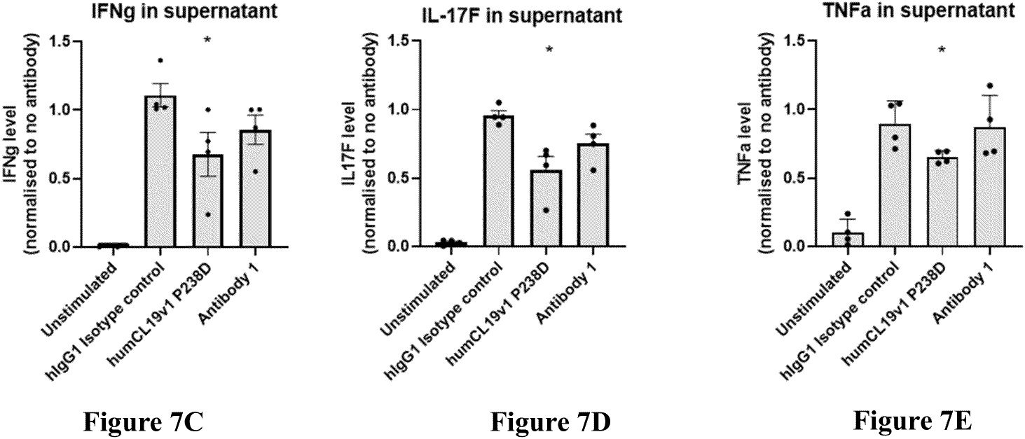

In flat 96-well plates 10,000 FLS cells were plated out in 50 µl of media and left to adhere for 2 hours. Then 100,000 PBMCs were added in 50 µl of media. Anti-PD-1 antibodies (humCL19v1 P238D or Antibody 1 from WO 2019/168745 A1) or isotype control were added in 50µl media for a final concentration of 1 µg/ml. Finally anti-CD3 (clone OKT3) and anti-CD28 (clone CD28.2) were added in 50 µl of media for a final concentration of 0.5 ng/ml each. After 3 days incubation at 37° C., 5%CO₂ supernatant was collected and assessed by cytometric bead array (Biolegend Th17 Panel # 741032) and cells were assessed by flow cytometry with the markers CD3,CD4,CD25 and ICOS. The agonist humCL19v1 P238D led to a significant reduction in T cell activation markers on CD4 T cells including CD25 (FIG. 7A) and ICOS (FIG. 7B), as well as a significant reduction in inflammatory cytokine production including IFNg (FIG. 7C), IL17F (FIG. 7D) and TNFa (FIG. 7E). The reference agonist Antibody 1 did not have a significant impact on any of these readouts. These data suggest that humCL19v1 P238D can be active in settings where PD-1′s ligands are expressed, whereas other described PD-1 agonists might be ineffective in these settings.

### Example 10. HumCL19v1 P238D Enhances the Interaction of PD-L1 with PD-1

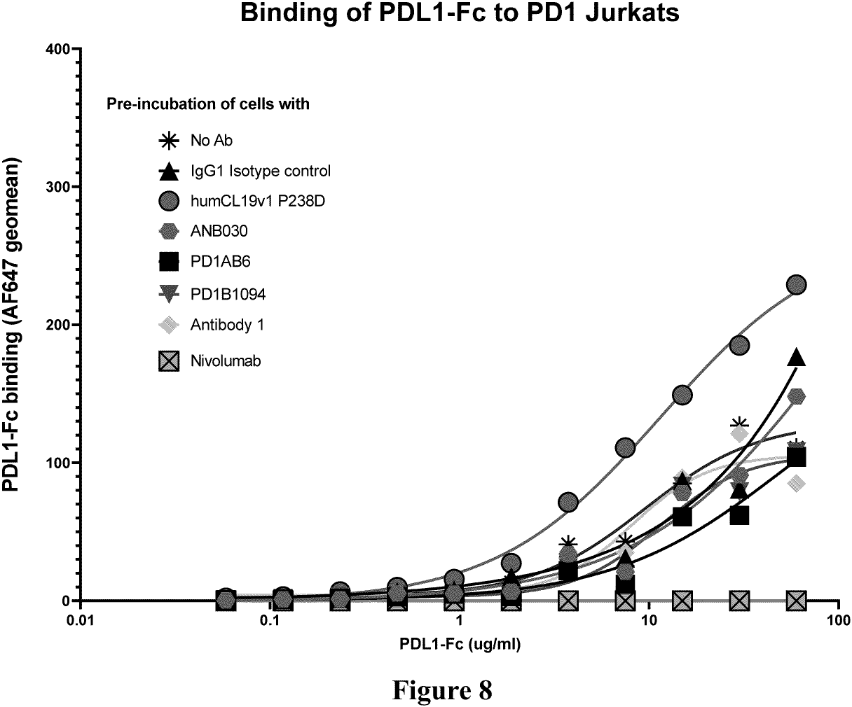

The binding of PD-L1 to PD-1 expressing cells was assessed in the presence of various PD-1 antibodies. PD-1 expressing Jurkat T cells were incubated with PD-1 antibody or isotype control at a concentration of 10 µg/ml on ice for 1 hour. Cells were then washed and incubated for 30 minutes with increasing concentrations of PDL1-Fc (Biolegend #762506) which had been conjugated to AF647 using a conjugation kit (Thermofisher #A20186). Cells were then washed again and assessed by flow cytometry. Cells that had been pre-incubated with humCL19v1 P238D demonstrated brighter staining with PDL1-Fc than cells that had been pre-incubated with no antibody, isotype control or other described PD-1 antibodies (FIG. 8). Cells pre-incubated with Nivolumab demonstrated no binding to PDL1-Fc as expected due to its ligand blocking epitope. These data suggest that humCL19v1 binding to PD-1 enhances its interaction with PD-L1.

### Example 11. Genes Downregulated by PD-1 Agonist Are Associated with Autoimmunity

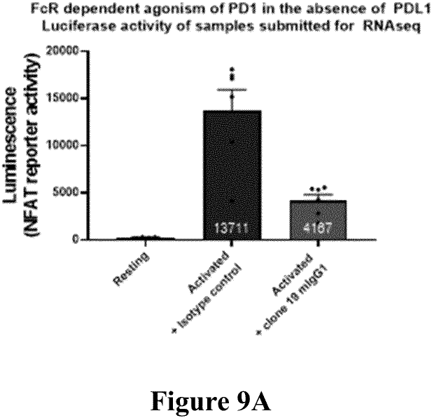

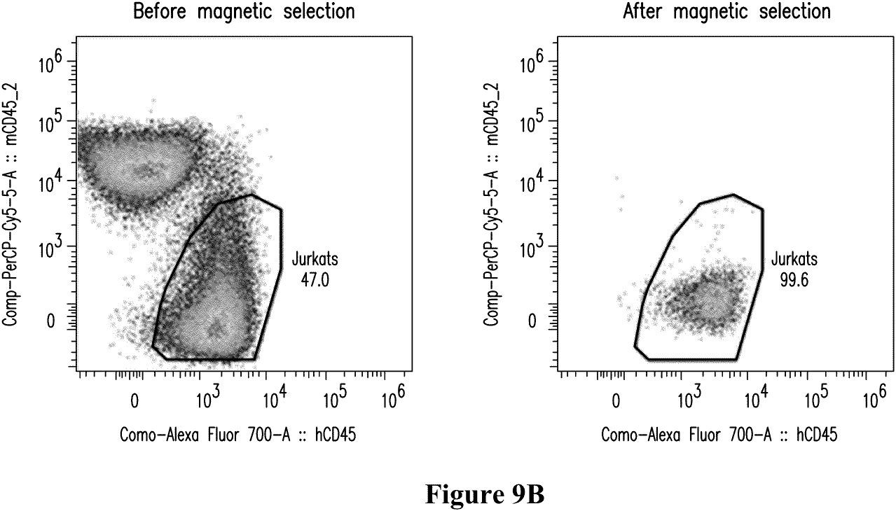

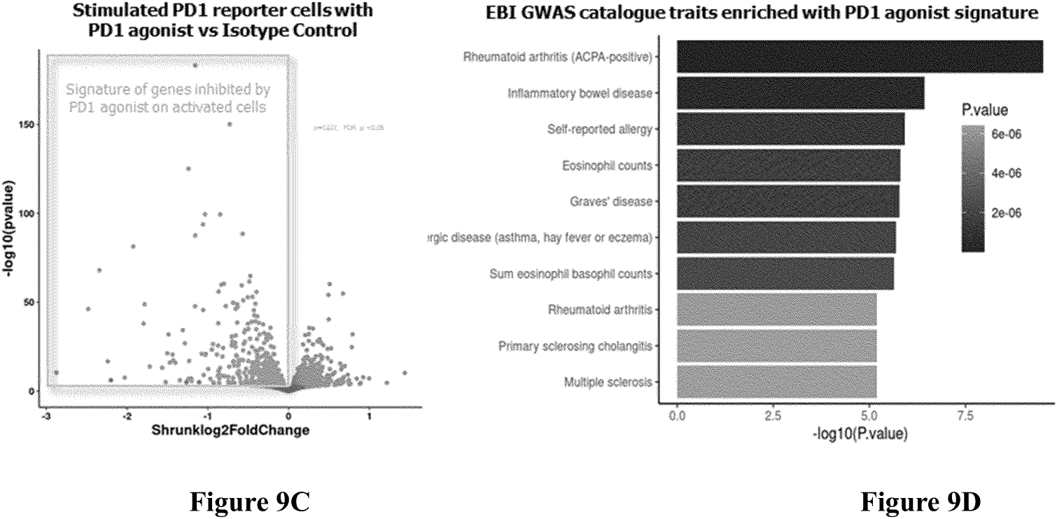

Methods similar to those describe in Example 1 were used to define the transcriptional signature of PD-1 agonism by Clone 19. 1.5million PD-1 expressing reporter Jurkats were added per well of a 6 well flat bottom plate in 1ml assay buffer (RPMI 1% FCS), containing 10 µg/ml of test antibody, then 1.5million FcR expressing stimulator cells were added in 1 ml assay buffer (to produce a final antibody concentration of 5 µg/ml). For the resting samples 1.5 million Jurkats were added in 2 ml assay buffer to empty wells. All conditions were performed with 6 technical replicates. All samples were incubated at 37C for 18 hours. 80 µl of cells from each sample was then transferred to a white 96 well plate for luciferase assessment (as described in Example 1) (FIG. 9A), 80 µl was transferred to a 96 well plate for flow cytometry assessment and then from the remaining sample stimulator cells were depleted by negative selection using Mojosort mouse CD45 nanobeads (Biolegend #480028). From the remaining cells 80 µl was transferred to a 96 well plate for flow cytometry assessment to check Jurkat cell purity (FIG. 9B). The remainder was pelleted by centrifugation, supernatant was aspirated then cells were frozen at -80. Samples underwent RNA extraction and sequencing at GeneWiz using a stranded library preparation protocol with a sequencing depth target of at least 20 M reads per sample.

RNA sequencing files from GeneWiz were processed using the rnaseq (v3.1) Nextflow pipeline within nf-core. FastQC was used to confirm the quality of sequencing and Salmon used to enumerate transcripts against the human genome (GRCh38 v96). An in-house differential expression pipeline was used to perform all subsequent analyses, using tximport to generate gene counts from estimated transcript abundances, DESeq2 to perform differential expression between groups (without adjusting for any additional covariates), and the EnrichR package to perform gene set enrichment analyses for sets of genes significantly higher or lower in expression between groups; using a variety of different gene set databases via Enrichr. Sets of genes significantly higher or lower is a group were defined based on their log-fold change in the DESeq2 analysis where a gene FDR correct p-value was less than 0.05. 1227 genes were significantly downregulated in cells activated in the presence of PD-1 agonist, compared to activation in the presence of isotype control (FIG. 9C). Mapping this collection of downregulated genes onto the EBI GWAS catalogue revealed an enrichment of genes associated with autoimmune disease, in particular sero-positive rheumatoid arthritis (FIG. 9D). These data suggest that antibody agonism of the PD-1 pathway can downregulate inflammatory genes pathways associated with autoimmunity.

### Example 12. Treatment of A Mouse Model of Systemic Lupus Erythematosus

The efficacy of exemplary antibody Clone 19 in treating an SLE disease model was tested in a transfer model where disease was induced in H2-Ab1bm12 recipient mice by the transfer of partially MHC-II mismatched splenocytes from humanized PD-1 mice. Systemic Lupus Erthyematosus (SLE) is a chronic autoimmune disease characterized by a breakdown in self-tolerance and the production of auto-antibodies against nuclear antigens such as chromatin and DNA. The deposition of immune complexes in various organs, including the kidneys and skin, results in diverse clinical presentations. It can affect approximately 0.1% of the population with increased frequency in females of child-bearing age and in certain ethnicities including African-Americans, Asians, Hispanics, and Native Americans (Izmirly et al., 2021). Existing therapies include anti-inflammatories and immunosuppressive agents like corticosteroids, which can help manage symptoms, but there is no cure for disease and there remains a need for more effective treatments.

The efficacy of Clone 19 in ameliorating disease was tested in a mouse model of SLE. The transfer model of SLE has previously been described in which disease is induced by the transfer of unfractionated splenocytes from donor bm12 mice to C57BL/6 recipient mice, or vice versa (Klarquist & Janssen, 2015). Bm12 mice differ from C57BL/6 mice by 3 amino acids in the MHC class II antigen H-2A. This MHC mismatch leads to an allogeneic response in which donor CD4 T cells become activated, differentiate into follicular helper cells (Tfh) and drive germinal center formation with the production of autoantibodies. An adapted protocol was used in which donor splenocytes were derived from C57BL/6 mice humanized at the PD-1 gene locus, to enable assessment of the anti-human PD-1 agonist antibody Clone 19. These mice have previously been described and characterized in the PhD Thesis of Billur Akkaya (Akkaya, 2012). This provides a model in which the disease-initiating T-cells are targetable with anti-human antibody, although some disease-propagating immune cells (such as B cells of the recipient) do not express the human PD-1.

Donor C57BL/6 mice, humanized at the PD-1 gene loci, were sacrificed and spleens harvested into RPMI media + 2%FCS + P/S/N. bm12 mice were also sacrificed to provide donor splenocytes for disease free controls. Spleens were processed to single cell suspension by pressing through 70 µM nylon cell strainers using the plunger of a 5ml syringe. Cells were then pelleted and resuspended in PBS at a concentration of 200 million cells per ml. 200 µl of this cell suspension was then injected intraperitoneally per recipient mouse (40 million cells per mouse). Adult bm12 mice (Jax Stock No: 00116) were used as recipients. Female recipients received cells from female donors and male recipients received cells from male donors. Test and control antibodies were diluted to 1 mg/ml in sterile PBS and injected IP. Groups were distributed between cages to avoid confounding cage effects. Dexamethasone was administered to the control group in drinking water (so these mice had to be grouped into the same cages). In order to provide approx. 0.5 mg/kg/day, an average daily intake of 0.2 ml/g was assumed. Dexamethasone was first reconstituted at 10 mg/ml in 100% ethanol then diluted to 2.5 µg/ml (1 in 4000) in drinking water.

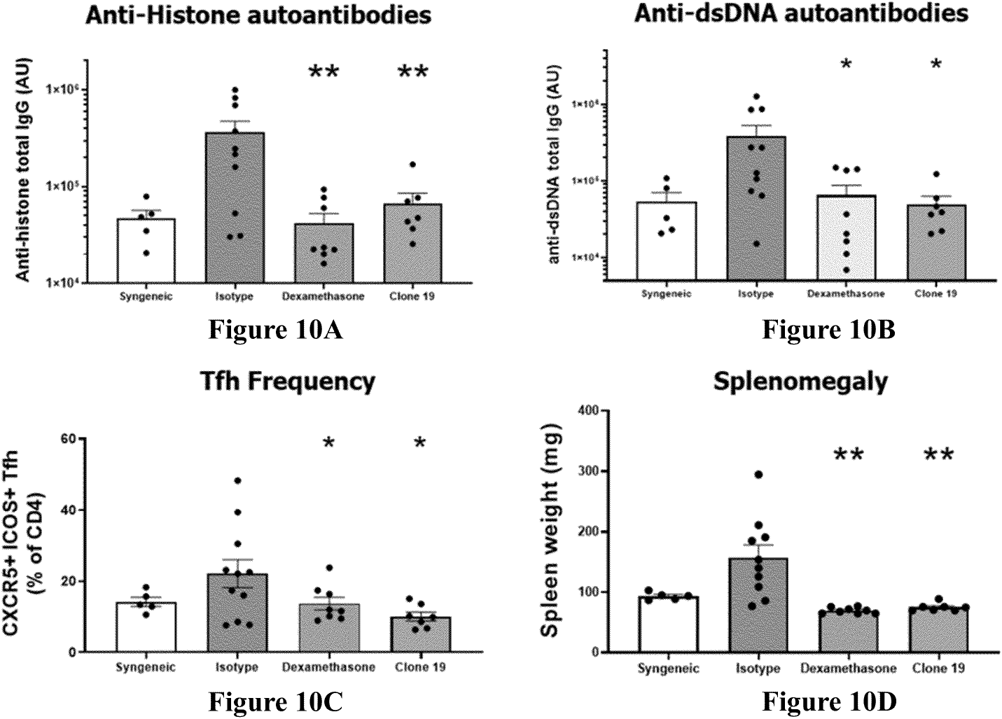

In a first study, mice were dosed intraperitoneally with 200 µg test antibody or mIgG1 isotype control on days 1 and 22 after cell transfer. A control group was administered Dexamethasone in drinking water from day 1 until termination on day 35. Mice were bled by tail vein venesection pre-dose on day 22 for serum auto-antibody ELISAs and on day 35 when the study was terminated. Spleens were also collected and assessed by flow cytometry to quantify the immune cell expansion, including expansion of donor Tfh cells (gated as CD4+CXCR5+ICOS+ cells). Clone 19 led to a near complete prevention of disease as assessed by autoantibody levels (FIGS. 10A-10B), Tfh frequency (FIG. 10C) or splenomegaly (FIG. 10D). The effect size was equivalent to dexamethasone administered throughout the study. There was a high degree of variability between untreated mice with some failing to engraft donor cells. The effect size was equivalent to dexamethasone administered throughout the study.

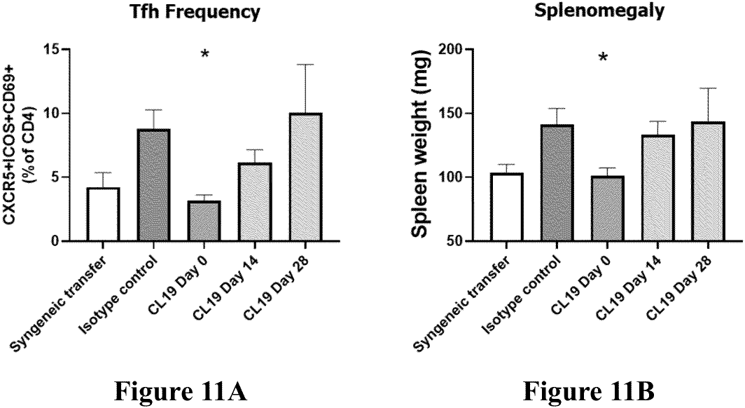

In another experiment, Clone 19 was administered at different time points, administration on day 0 again completely prevented the expansion of donor TfH and significantly reduced other markers of disease development (FIGS. 11A-11B). Administration on day 14 led to a partial reduction in disease markers. Administration on day 28 (before study termination on day 30) did not lead to a significant reduction in any markers, including Tfh cell frequency. As the Tfh cells express high levels of human PD-1 it would be expected that they would be significantly depleted within this 48 hour timeframe if Clone 19 was acting as a depleting antibody via ADCC or CDC.

### Example 13. PD-1 Agonist Inhibits The Delayed Type Hypersensitivity Response In Humanized Mice

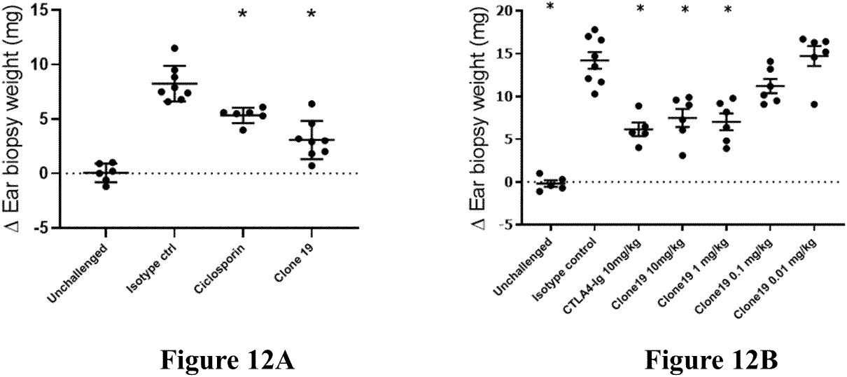

C57BL/6 mice humanized at the PD-1 gene locus were used to assess the impact of the PD-1 agonist Clone 19 on the delayed type hypersensitivity response in a skin challenge model relevant to autoimmune skin diseases. On Day 0 mice were immunized with keyhole limpet hemocyanin (KLH) in Complete Freund Adjuvant (CFA). The emulsion was a mixture of KLH (Sigma) in PBS, added to CFA (BD Biosciences), at a ratio of 1:1. The final concentration of KLH was 4 mg/mL. Animals were immunized with 100 µL of immunization emulsion injected subcutaneously in 1-2 sites. The unchallenged control group received just PBS. Also on day 0 mice were treated, 1 h prior to the immunization, with either mIgG1 Isotype control (clone Mopc21) or anti-PD-1 clone 19 mIgG1 intraperitoneally, at a single dose of 10 mg/kg. The unchallenged control group received just PBS. Animals in the positive treatment control group were treated by oral gavage with CsA at a dose of 3 mg/kg once per day from Day 0-5. To prepare CsA, Sandimmune Neoral solution (Novartis) was diluted to 0.3 mg/ml in 0.5% methylcellulose 400cp (Sigma). Five days after immunization, mice were challenged in the pinna of the left ear (under anesthetic) with 20 µL of 4 mg/mL antigen solution. The unchallenged control group received 20 µL of PBS in the pinna of the left ear. One day after ear challenge, ear thickness was measured using digital calipers. After measuring ear thickness, animals were humanely sacrificed and, postmortem, an 8 mm diameter circle was cut using a biopsy punch from the left and right ear of each animal from all groups. Ears were weighed on a precise analytical balance. Ear oedema was assessed as the difference between left (challenged) and right (control) ear weight. The PD-1 agonist Clone 19 led to a significant inhibition in ear swelling (FIG. 12A).

In another experiment, mice were treated on day 0, 1 h prior to the immunization, with either 10, 1, 0.1, or 0.01 mg/kg Clone 19 intraperitoneally. In this study a CTLA-Ig fusion protein (Biolegend, cat# 591908) was used as the positive control, administered at a dose of 10 mg/kg IP 1 h prior to the immunization on day 0. Clone 19 at doses of 10 mg/kg or 1 mg/kg led to a significant inhibition of ear swelling, comparable to the effect of CTLA4-Ig (FIG. 12B).

### Example 14. PD-1 Agonist Ameliorates Symptoms of Graft-Versus-Host Disease In Mouse Model

In one set of experiments, the impact of humCL19v1 P238D on human PBMC-driven graft-versus-host disease (GvHD) was determined in vivo. Briefly, female NSG mice (JAX Labs, Stock # 05557), approximately 8-10 weeks old, were irradiated with 2.4 Gy total body irradiation (on day -1). Human peripheral blood mononuclear cells (PBMCs) were isolated from a leukopak (a HemaCare product ordered via Tissue Solutions) and resuspended at 25 × 10⁶ cells per ml of PBS. Mice were injected with 200 µl cell suspension (5 × 10⁶ PBMCs) intravenously (IV) by tail injection 1 day after irradiation (on day 0). Also on day 0, and on days 7, 14 and 21, mice were treated with 10 mg/kg of humCL19v1 P238D or P238D mutated hIgG1 isotype control by intraperitoneal injection. Mice were weighed regularly and euthanised when they had lost 15% body weight or after 28 days. At study termination peripheral blood was collected for assessment of inflammatory cytokines by cytometric bead array. Spleens were weighed and infiltration of human PBMCs into liver and spleen was quantified by flow cytometry using markers for hCD45, hCD4, hCD8, hCD20, hCD25 and FOXP3. Production of IFNg by CD8 and CD4 T cells in spleen and liver was also assessed by intracellular flow cytometry.

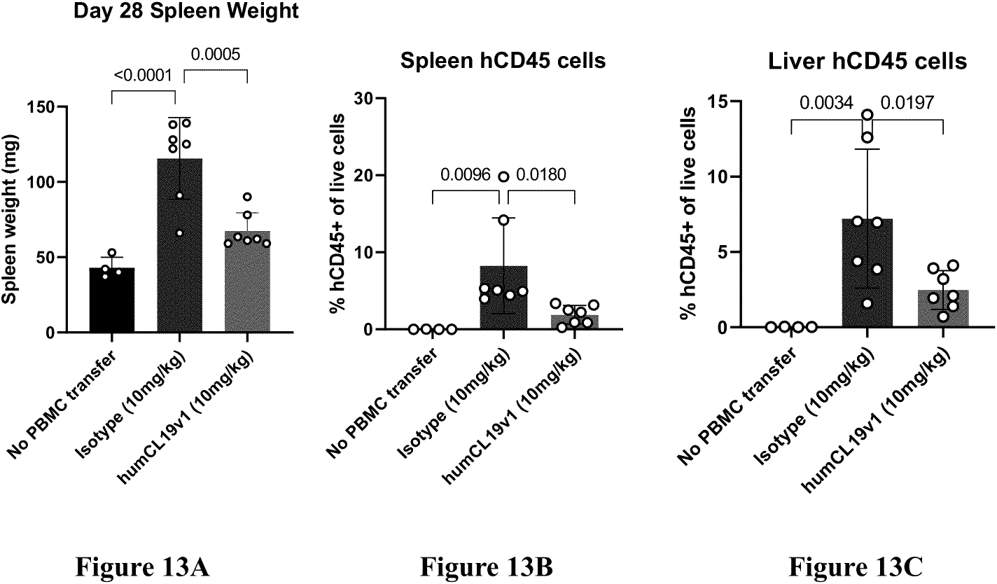

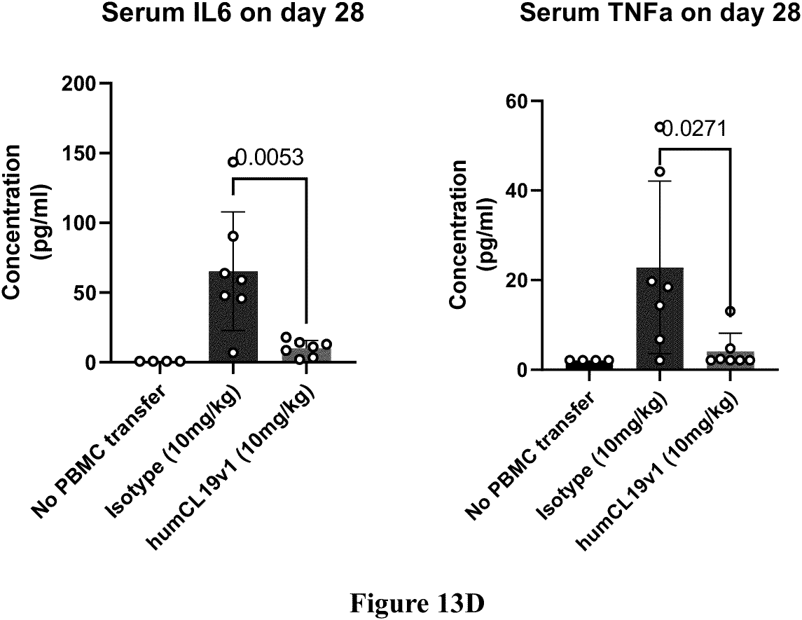

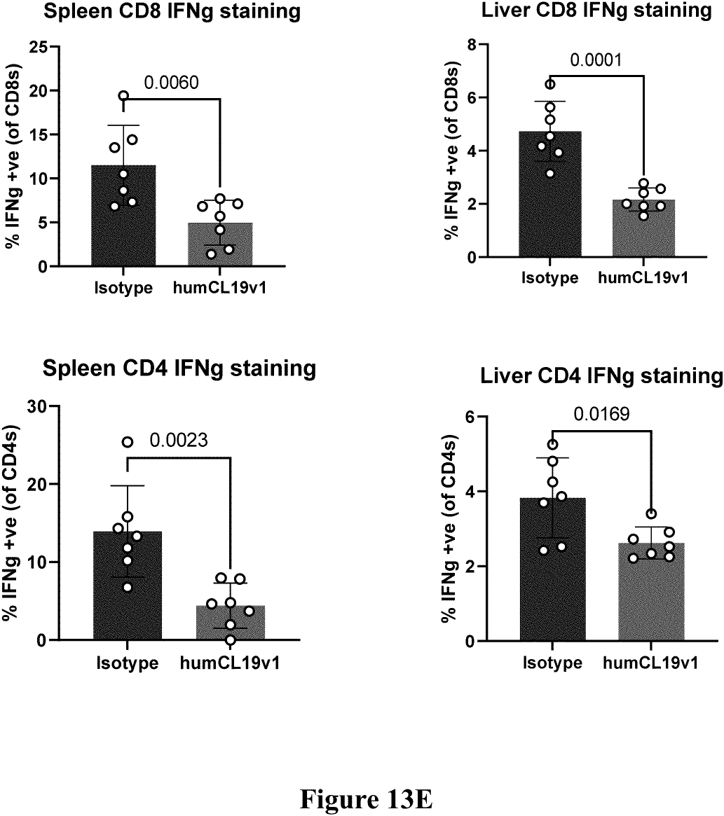

Following procedures as described above humCL19v1 P238D significantly reduced spleen weights (FIG. 13A), human immune cell expansion in spleen (FIG. 13B) and liver (FIG. 13C), and serum inflammatory cytokine levels (FIG. 13D) compared to isotype control. Furthermore, in addition to reducing the overall cytokine levels, humCL19v1 P238D also reduced CD4 and CD8 cytokine production on a per cell basis, as assessed by intracellular flow cytometry of human immune cells in the liver and spleen (FIG. 13E).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method of suppressing an immune cell that expresses Programmed death 1 (PD-1), comprising contacting the immune cell with an antibody that specifically binds to PD-1 and (a) agonizes PD-1 signaling in the immune cell, or (b) enhances interaction of the PD-1 on the surface of the immune cell with PD-L1.

2. (canceled)

3. The method of claim 1, wherein the antibody comprises an Fc region, and wherein the Fc region comprises an amino acid substitution, wherein the amino acid substitution results in reduced antibody-dependent cellular cytotoxicity (ADCC) against a PD-1 expressing regulatory T cell in the subject compared to a parent molecule that lacks the amino acid substitution, and wherein the antibody has the same or higher agonistic effect on PD-1 signaling in the immune cell compared to the parent molecule.

4. (canceled)

5. The method of claim 1, wherein the ADCC against the PD-1 expressing regulatory T cell is reduced as determined by a natural killer cell activation assay as described in Example 7.

6. The method of claim 1, wherein the antibody
(i) does not lead to significant ADCC against the PD-1 expressing regulatory T cell, as determined by a natural killer cell activation assay as described in Example 7;
(ii) does not activate natural killer (NK) cells;
(iii) comprises a heavy chain that comprises a heavy chain variable, and a light chain that comprises a light chain variable region, optionally wherein the heavy chain variable comprises a complementarity determining region (CDR) comprising the sequence as set forth in one or more of SEQ ID NOs: 1-3, with 0 to 3 amino acid modifications, and the light chain variable region comprises a CDR comprising the sequence as set forth in one or more of SEQ ID NOs: 4-6, with 0 to 3 amino acid modifications; and/or
(iv) the Fc region is derived from an IgG1 and comprises aspartic acid (D) at position 238 numbered according to EU index.

7-10. (canceled)

11. The method of claim 1, wherein the antibody comprises a heavy chain, a light chain, and an Fc region, wherein:
(i) the heavy chain comprises a heavy chain variable region that comprises a CDR comprising the sequence as set forth in one or more of SEQ ID NOs: 1-3, with 0 to 3 amino acid modifications;
(ii) the light chain comprises a light chain variable region that comprises a CDR comprising the sequence as set forth in one or more of SEQ ID NOs: 4-6, with 0 to 3 amino acid modifications; and
(iii) the Fc region is derived from an IgG1 and comprises aspartic acid (D) at position 238 numbered according to EU index.

12. (canceled)

13. The method of claim 1, wherein
(i) the heavy chain variable region comprises heavy chain complementarity determining region 1 (CDRH1), CDRH2, and CDRH3, and wherein CDRH1, CDRH2, and CDRH3 comprise the sequence as set forth in SEQ ID NOs: 1-3, respectively, with 0 to 3 amino acid modifications;
(ii) the light chain variable region comprises light chain complementarity determining region 1 (CDRL1), CDRL2, and CDRL3, and wherein CDRL1, CDRL2, and CDRL3 comprise the sequence as set forth in SEQ ID NOs: 4-6, respectively, with 0 to 3 amino acid modifications;
(iii) the heavy chain variable region comprises CDRH1, CDRH2, and CDRH3, and wherein CDRH1, CDRH2, and CDRH3 comprise the sequence as set forth in SEQ ID NOs: 1-3, respectively;
(iv) the light chain variable region comprises CDRL1, CDRL2, and CDRL3, and wherein CDRL1, CDRL2, and CDRL3 comprise the sequence as set forth in SEQ ID NOs: 4-6, respectively;
(v) the heavy chain variable region comprises a sequence that has at least 80%, 85%, 90%, 95%, or 99%, or 100% identity to the sequence as set forth in any one of SEQ ID NOs: 7-11;
(vi) the light chain variable region comprises a sequence that has at least 80%, 85%, 90%, 95%, or 99%, or 100% identity to the sequence as set forth in any one of SEQ ID NOs: 12-16;
(vii) the heavy chain comprises a sequence that has at least 80%, 85%, 90%, 95%, or 99%, or 100% identity to the sequence as set forth in SEQ ID NO: 18;
(viii) the light chain comprises a sequence that has at least 80%, 85%, 90%, 95%, or 99%, or 100% identity to the sequence as set forth in SEQ ID NO: 19; and/or
(ix) the heavy chain variable region and the light chain variable region form a structure selected from the group consisting of: scFv, sc(Fv)2, dsFv, Fab, Fab′, (Fab′)2 and a diabody.

14-20. (canceled)

21. The method of claim 3, wherein
(i) the Fc region is derived from a human IgG1 and/or the Fc region comprises a sequence that has at least 80%, 85%, 90%, 95%, or 99%, or 100% identity to the sequence as set forth in SEQ ID NO: 17;
(ii) the immune cell comprises a T cell, a B cell, or a macrophage, or an antigen-specific T cell;
(iii) the Fc region selectively binds to FcγR2B, optionally the antibody binds to human FcγR2B with a KD of less than 5 µM, 4 µM, 3 µM, or 2 µM, as determined by surface plasmon resonance at 37° C.

22. (canceled)

23. (canceled)

24. The method of claim 1, wherein
(i) the heavy chain variable region and the light chain variable region form a single-chain variable fragment (ScFv) that is operably linked to the Fc region;
(ii) the antibody is selected from the group consisting of: a human antibody, a humanized antibody, a chimeric antibody, and a multispecific antibody;
(iii) the antibody is monoclonal;
(iv) the antibody decreases activation of the immune cell by at least about 10%, 15%, 20%, 25%, 30%, 40%, or 50%; and/or
(v) the antibody decreases activation of the immune cell by from about 10% to 50%, 10% to 40%, 10% to 30%, 10% to 20%, 10% to 15%, 20% to 50%, 20% to 40%, or 20% to 30%.

25-32. (canceled)

33. The method of claim 21, wherein
(i) the antibody binds to human FcγR2A (131R allotype) with a K_{D} of more than 5 µM or 10 µM, or at least 15 µM, as determined by surface plasmon resonance at 37° C.;
(ii) the antibody binds to human FcγR2A (131H allotype) with a KD of at least 50 µM or at least 80 µM, as determined by surface plasmon resonance at 37° C.;
(iii) a ratio of binding affinity of the antibody to human FcγR2B versus binding affinity of the antibody to human FcγR2A (131R allotype) is at least 2:1, 3:1, 4:1, 5:1, or 6:1, or at least 6:1, or about 6:1, as determined by surface plasmon resonance at 37° C.; and/or
(iv) a ratio of binding affinity of the antibody to human FcγR2B versus binding affinity of the antibody to human FcγR2A (131H allotype) is at least 10:1, 15:1, 20:1, 40:1, or 50:1, or at least 40:1, or about 40:1, as determined by surface plasmon resonance at 37° C.

34-43. (canceled)

44. An isolated antibody that specifically binds to Programmed death 1 (PD-1) and agonizes PD-1 signaling, wherein the antibody comprises a heavy chain, a light chain, and an Fc region,
wherein the heavy chain comprises a heavy chain variable region,
wherein the light chain comprises a light chain variable region,, and
wherein the antibody (a) has the same or higher agnostic effect on PD-1 signaling in an immune cell compared to the parent molecule or (b) enhances interaction of PD-1 expressed on the surface of an immune cell with PD-L1.

45. (canceled)

46. The antibody of claim 44, wherein the Fc region comprises an amino acid substitution that results in reduced antibody-dependent cellular cytotoxicity (ADCC) against a PD-1 expressing regulatory T cell in the subject compared to a parent molecule that lacks the amino acid substitution, and wherein the antibody has the same or higher agnostic effect on PD-1 signaling in an immune cell compared to the parent molecule.

47. The antibody of claim 44, wherein
the heavy chain variable region comprises a CDR comprising the sequence as set forth in one or more of SEQ ID NOs: 1-3, with 0 to 3 amino acid modifications;
the light chain variable region comprises a CDR comprising the sequence as set forth in one or more of SEQ ID NOs: 4-6, with 0 to 3 amino acid modifications; and/or
the Fc region is derived from an IgG1 and comprises aspartic acid (D) at position 238 numbered according to EU index.

48-51. (canceled)

52. The antibody of claim 46, wherein the interaction between PD-1 and PD-L1 is enhanced as determined by an assay as described in Example 10.

53. The antibody of claim 47, wherein the antibody induces reduced antibody-dependent cellular cytotoxicity (ADCC) against a PD-1 expressing regulatory T cell compared to an otherwise same molecule that comprises an Fc region of the IgG1, and wherein the antibody has the same or higher agnostic effect on PD-1 signaling in an immune cell compared to the otherwise same molecule.

54. The antibody of claim 46, wherein
(i) the ADCC against the PD-1 expressing regulatory T cell is reduced as determined by a natural killer cell activation assay as described in Example 7;
(ii) the antibody does not lead to significant ADCC against the PD-1 expressing regulatory T cell, as determined by a natural killer cell activation assay as described in Example 7; and/or
(iii) the antibody does not activate natural killer (NK) cells.

55. (canceled)

56. (canceled)

57. The antibody of claim 44, wherein
(i) the heavy chain variable region comprises heavy chain complementarity determining region 1 (CDRH1), CDRH2, and CDRH3, and wherein CDRH1, CDRH2, and CDRH3 comprise the sequence as set forth in SEQ ID NOs: 1-3, respectively, with 0 to 3 amino acid modifications;
(ii) the light chain variable region comprises light chain complementarity determining region 1 (CDRL1), CDRL2, and CDRL3, and wherein CDRL1, CDRL2, and CDRL3 comprise the sequence as set forth in SEQ ID NOs: 4-6, respectively, with 0 to 3 amino acid modifications;
(iii) the heavy chain variable region comprises CDRH1, CDRH2, and CDRH3, and wherein CDRH1, CDRH2, and CDRH3 comprise the sequence as set forth in SEQ ID NOs: 1-3, respectively;
(iv) the light chain variable region comprises CDRL1, CDRL2, and CDRL3, and wherein CDRL1, CDRL2, and CDRL3 comprise the sequence as set forth in SEQ ID NOs: 4-6, respectively;
(v) the heavy chain variable region comprises a sequence that has at least 80%, 85%, 90%, 95%, or 99%, or 100% identity to the sequence as set forth in any one of SEQ ID NOs: 7-11;
(vi) the light chain variable region comprises a sequence that has at least 80%, 85%, 90%, 95%, or 99%, or 100% identity to the sequence as set forth in any one of SEQ ID NOs: 12-16;
(vii) the heavy chain comprises a sequence that has at least 80%, 85%, 90%, 95%, or 99%, or 100% identity to the sequence as set forth in SEQ ID NO: 18;
(viii) the light chain comprises a sequence that has at least 80%, 85%, 90%, 95%, or 99%, or 100% identity to the sequence as set forth in SEQ ID NO: 19;
(ix) the Fc region is derived from a human IgGl;
(x) the Fc region comprises a sequence that has at least 80%, 85%, 90%, 95%, or 99%, or 100% identity to the sequence as set forth in SEQ ID NO: 17;
(xi) (a) the heavy chain variable region and the light chain variable region form a structure selected from the group consisting of: scFv, sc(Fv)2, dsFv, Fab, Fab′, (Fab′)2 and a diabody; (b) the antibody comprises a heavy chain and a light chain, wherein the heavy chain comprises the heavy chain variable region operably linked to the Fc region, and wherein the light chain comprises the light chain variable region; or (c) the heavy chain variable region and the light chain variable region form a single-chain variable fragment (ScFv) that is operably linked to the Fc region;
(xii) the antibody is a humanized antibody; a human antibody; or selected from the group consisting of: a human antibody, a humanized antibody, a chimeric antibody, and a multispecific antibody;
(xiii) the antibody is monoclonal;
(xiv) the antibody binds human PD-1 with a K_{D} of (a) less than 200 nM, 100 nM, 80 nM, 60 nM, or 40 nM, as determined by surface plasmon resonance (SPR) at 37° C.; (b) less than 60 nM as determined by surface plasmon resonance (SPR) at 37° C.; or (c) less than 40 nM as determined by surface plasmon resonance (SPR) at 37° C.;
(xv) the antibody binds cynomolgus PD-1 with a KD of (a) less than 5000 nM, 4000 nM, 2000 nM, 1000 nM, 800 nM, 600 nM, 500 nM, 400 nM, 300 nM, or 200 nM as determined by surface plasmon resonance (SPR) at 37° C.; (b) less than 600 nM as determined by surface plasmon resonance (SPR) at 37° C.; or (c) less than 300 nM as determined by surface plasmon resonance (SPR) at 37° C.; and/or
(xvi) the antibody agonizes human PD-1 expressed on the surface of an immune cell, optionally wherein the immune cell is a T cell.

58-81. (canceled)

82. The antibody of claim 57, wherein binding of the antibody to human PD-1 expressed on the surface of an immune cell decreases proliferation of the cell relative to a comparable immune cell not bound by the antibody, optionally wherein the cell is a T cell.

83. (canceled)

84. The antibody of claim 82, wherein
(i) the decrease in cell activation is measured by an NFAT-reporter assay described in Example 4;
(ii) the decrease in cell activation is measured by a Tetanus Toxoid activation assay or a viral peptide activation assay described in Example 5;
(iii) when the immune cell is in proximity of PD-L1 expressing cells, optionally wherein the decrease in cell proliferation is measured by an assay described in Example 8; or
(iv) the decrease in cell proliferation is measured in vitro or in vivo.

85-89. (canceled)

90. The antibody of claim 82, wherein the decrease in cell proliferation is at least about 10%, 15%, 20%, 25%, 30%, 40%, or 50%; or the decrease in cell proliferation is from about 10% to 50%, 10% to 40%, 10% to 30%, 10% to 20%, 10% to 15%, 20% to 50%, 20% to 40%, or 20% to 30%..

91. (canceled)

92. The antibody of claim 57 wherein the Fc region selectively binds to FcγR2B.

93. The antibody of claim 92, wherein
(i) the antibody binds to human FcγR2B with a K_{D} of less than 5 µM, 4 µM, 3 µM, or 2 µM, as determined by surface plasmon resonance at 37° C., and/or the antibody binds to human FcγR2B with a KD of at least 2 µM, 1 µM, 800 nM, 600 nM, 500 nM, 400 nM, 300 nM, 200 nM, 100 nM, 80 nM, 60 nM, 50 nM, 40 nM, 30 nM, 20 nM, 10 nM, or 5 nM, or
(ii) the antibody binds to human FcγR2B with a KD of 200 nM to 5 µM, 400 nM to 4 µM, 500 nM to 3.5 µM, 800 nM to 3 µM, 1 µM to 5 µM, 1 µM to 4.5 µM, 1 µM to 4 µM, 1 µM to 3.5 µM, 1 µM to 3 µM, 1 µM to 2.5 µM, or 1 µM to 2 µM;
(iii) the antibody binds to human FcγR2A (131R allotype) with a K_{D} of more than 5 µM or 10 µM, as determined by surface plasmon resonance at 37° C., or the antibody binds to human FcγR2A (131R allotype) with a KD of at least 15 µM, as determined by surface plasmon resonance at 37° C.; and/or
(iv) the antibody binds to human FcγR2A (131H allotype) with a K_{D} of at least 50 µM, as determined by surface plasmon resonance at 37° C., or the antibody binds to human FcγR2A (131H allotype) with a KD of at least 80 µM, as determined by surface plasmon resonance at 37° C.

94-99. (canceled)

100. The antibody of claim 92, wherein a ratio of binding affinity of the antibody to human FcγR2B versus binding affinity of the antibody to human FcγR2A (131R allotype), as determined by surface plasmon resonance at 37° C.,
(i) is at least 2:1, 3:1, 4:1, 5:1, or 6:1, or at least 6:1, or about 6:1; and/or
(ii) is at least 10:1, 15:1, 20:1, 40:1, or 50:1, or at least 40:1, or about 40:1.

101-106. (canceled)

107. An isolated nucleic acid that comprises one or more nucleotide sequences encoding polypeptides capable of forming the antibody of claim 57.

108. A vector that comprises one or more nucleotide sequences encoding polypeptides capable of forming the antibody of claim 107.

109. A host cell comprising the vector of claim 108.

110. A method, comprising culturing the host cell of claim 109 under conditions for production of the antibody.

111. A method, comprising
isolating the antibody of claim 110.

112. An immunoconjugate comprising the antibody of claim 57 conjugated with an agent.

113. A pharmaceutical composition comprising a therapeutically effective amount of the antibody of claim 57 or an immunoconjugate comprising the antibody conjugated with an agent, and at least one pharmaceutically acceptable excipient.

114. (canceled)

115. A kit comprising the antibody of claim 57 or an immunoconjugate comprising the antibody conjugated with an agent in a container.

116. The kit of claim 115, further comprising an informational material containing instructions for use of the antibody or the immunoconjugate.

117. A method of treating a disease or condition in a subject in need thereof, comprising administering to the subject a therapeutically effective amount of the antibody of any one of claims 57 or an immunoconjugate comprising the antibody conjugated with an agent, or administering to the subject the a pharmaceutical composition comprising a therapeutically effective amount of the antibody or the immunoconjugate, and at least one pharmaceutically acceptable excipient.

118. The method of claim 117, wherein
(i) the disease or condition comprises a disease or condition associated with PD-1,
(ii) the disease or condition comprises acute disseminated encephalomyelitis (ADEM), Addison’s disease, allergy, alopecia areata, amyotrophic lateral sclerosis, ANCA vasculitis, ankylosing spondylitis, anti-phospholipid syndrome, asthma, atopic dermatitis, autoimmune haemolytic anaemia, autoimmune hepatitis, autoimmune pancreatitis, autoimmune polyendocrine syndrome, Behcet’s disease, bullous pemphigoid, cerebral malaria, chronic inflammatory demyelinating polyneuropathy, coeliac disease, Crohn’s disease, Cushing’s Syndrome, dermatitis herpetiformis, dermatomyositis, diabetes mellitus type 1, eosinophilic granulomatosis with polyangiitis, gallbladder disease, graft versus host disease, Graves’ disease, Guillain-Barre syndrome, Hashimoto’s thyroiditis, Hidradenitis Suppurativa, IgG4-related disease, inflammatory bowel disease (IBD), inflammatory fibrosis, irritable bowel syndrome, juvenile arthritis, Kawasaki disease, leukemia, lupus nephritis, lyme arthritis, lymphoma, lymphoproliferative disorders, meningoencephalitis, multiple sclerosis, myasthenia gravis, myeloma, non-radiographic axial spondyloarthritis (nr-AxSpA), neuromyelitis optica, osteoarthritis, pelvic inflammatory disease, pemphigus, peritonitis, Pilonidal disease, polymyositis, primary biliary cholangitis, primary sclerosing cholangitis, psoriasis, psoriatic arthritis, rheumatoid arthritis, sarcoidosis, Sjögren’s syndrome, systemic lupus erythematosus, systemic sclerosis, Takayasu’s arteritis, temporal arteritis, transplant rejection, transverse myelitis, ulcerative colitis, uveitis, vasculitis, vitiligo and Vogt-Koyanagi-Harada Disease; and/or
(iii) the subject is a human subject.

119. (canceled)

120. (canceled)

121. A method of downregulating an immune response in a subject, comprising administering to the subject the antibody of claim 57, an immunoconjugate comprising the antibody conjugated with an agent, or administering to the subject a pharmaceutical composition comprising a therapeutically effective amount of the antibody or the immunoconjugate, and at least one pharmaceutically acceptable excipient.

122. A method of suppressing an immune cell that expresses PD-1, comprising contacting the immune cell with the antibody of claim 57 or an immunoconjugate comprising the antibody conjugated with an agent.

123. The method of claim 122, wherein
(i) the immune cell comprises a T cell, a B cell, a macrophage, or an antigen-specific T cell; and/or
(ii) the subject is a human subject.

124. (canceled)

125. (canceled)
